(19) United States
(12) Patent Application Publication
(10) Pub. No.: US 2010/0136114 A1
(43) Pub. Date: Jun. 3, 2010

(54) DE NOVO FORMATION AND REGENERATION OF VASCULARIZED TISSUE FROM TISSUE PROGENITOR CELLS AND VASCULAR PROGENTITOR CELLS

(75) Inventor: Jeremy J. Mao, Closter, NJ (US)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(73) Assignee: The Trustees of Columbia University in the City of New York, New York, NY (US)

(21) Appl. No.: 12/373,514

(22) PCT Filed: Jul. 10, 2007
(86) PCT No.: PCT/US07/15293

§ 371 Date: Jan. 12, 2010

Related U.S. Application Data
(60) Provisional application No. 60/824,597, filed on Sep. 5, 2006.
(60) Provisional application No. 60/819,833, filed on Jul. 10, 2006.

(51) Int. Cl. A61K 9/14; A61K 35/12; A61P 43/00

(52) U.S. Cl. 424/486; 424/93.7

(57) ABSTRACT

It has been discovered that vascularized tissue or organs can be engineered by combined actions of tissue progenitor cells and vascular progenitor cells. Provided herein are compositions and methods directed to engineered vascularized tissue or organs formed by introducing tissue progenitor cells and vascular progenitor into or onto a biocompatible scaffold of matrix material. Also provided are methods of treating tissue defects via grafting of such compositions into subjects in need thereof.

### CROSS-REFERENCE TO RELATED APPLICATIONS

This application claims priority from U.S. Provisional Application Ser. No. 60/819,833, filed on Jul. 10, 2006, and U.S. Provisional Application Ser. No. 60/824,597, filed on Sep. 5, 2006, each of which are incorporated herein by reference in their entirety.

### STATEMENT REGARDING FEDERALLY SPONSORED RESEARCH OR DEVELOPMENT

This invention was made in part with Government support under National Institute of Biomedical Imaging and Bioengineering and National Institute of Dental and Craniofacial Research Grant Nos. R01DE15291 and R01EB02332. The Government has certain rights in the invention.

### INCORPORATION-BY-REFERENCE OF MATERIAL SUBMITTED ON A COMPACT DISC

Not Applicable.

### FIELD OF THE INVENTION

The present invention generally relates to de novo formation and regeneration of vascularized tissues or organs from tissue progenitor cells and vascular progenitor cells.

### BACKGROUND

Clinical needs of tissue grafting for the recontruction of trauma, chronic diseases, tumor removal and congenital anomalies are substantial. Current surgical procedures rely on autologous grafts, allogenic grafts, xenogenic grafts or synthetic materials. The deficiencies associated with current clinical procedures are widely recognized in surgical and scientific communities.

The development of clinically transplantable three-dimensional engineered tissues or organs is limited by the fact that tissue assemblies greater than 100-200 μm require a perfused vascular bed to supply nutrients and to remove waste products, metabolic intermediates, and secreted products. Mature functional vascular networks have been difficult to engineer given that vascular development is a complex event involving various cell types and many different growth factors. During embryonic development, endothelial cells form tubes and connect to form the primary capillary plexus, a process termed angiogenesis. New vessels are formed by splitting existing vessels in two, or by sprouting from existing vessels. This primary network is remodeled and pruned in a process termed vessel maturation to form distinct microcirculatory units that include capillaries, arteries, and veins.

Suboptimal angiogenesis remains a critical roadblock in tissue engineering, especially for critical size tissue defects. Previous approaches in engineering angiogenesis have relied on the release of angiogenic growth factors, or the fabrication of blood vessel analogs. However, there are continuing concerns over the cost of growth factor delivery, potential toxicity, suboptimal anastomosis and slow endothelial migration for large tissue grafts.

Two subsets of stem cells can be isolated from a single bone marrow sample: mesenchymal stem cells (MSCs) and hematopoietic stem cells (HSCs). MSCs are capable of differentiating into virtually all connective tissue lineage cells. HSCs differentiate into endothelial cells, along with blood born cells that are essential to the formation of vascularized tissue.

Thus, there exists the need for compositions of engineered vascularized tissue constructs along with methods of producing such.

### SUMMARY

Disclosed herein is a new approach towards the engineering of vascularized tissue from combined actions of tissue progenitor cells and vascular progenitor cells. Vascularized tissue modules produced using the disclosed compositions and methods can be used in various clinical applications.

In some aspects, the invention is directed to a vascularized tissue module. In various configurations, a tissue module comprises a biocompatible matrix, tissue progenitor cells, and vascular progenitor cells. The progenitors cells can be introduced (e.g., by injection, endoscopy or infused, together or sequentially) into or onto a biocompatible scaffold of matrix material.

Another aspect of the invention provides a method for forming a vascularized tissue module. These methods include providing a biocompatible matrix, and introducing to the matrix both tissue progenitor cells and vascular progenitor cells. Progenitor cells can be delivered into or onto a biocompatible matrix material using methods well known in the art, such as by injection, endoscopy, or infusion. In various configurations, the delivery can be either simultaneous or sequential. The methods can further comprise incubating the matrix containing the tissue and vascular progenitor cells. In some configurations, tissue morphogenesis and/or cell differentiation can occur during the incubation. Such incubation can be at least in part in vitro, substantially in vitro, at least in part in vivo, or substantially in vivo. In some configurations, a module can be formed at least in part ex vivo, while in some other configurations, at least one of the biocompatible matrix, the tissue progenitor cells, and the vascular progenitor cells can be heterologous to an intended recipient such as a human in need of treatment for tissue repair or replacement.

In various aspects, tissue progenitor cells can be mesenchymal stem cells (MSCs), MSC-derived cells, osteoblasts, chondrocytes, myocytes, adipocytes, neuronal cells, cardiomyocytes, neural glial cells, Schwann cells, epithelial cells, dermal fibroblasts, interstitial fibroblasts, gingival fibroblasts, periodontal fibroblasts, cranial suture fibroblasts, tenocytes, ligament fibroblasts, uretheral cells, liver cells, periosteal cells, beta-pancreatic islet cells, or a combination thereof. In some configurations, the tissue progenitor cells can be, preferably, MSCs, MSC-derived cells, or a combination thereof.

In various aspects, vascular progenitor cells can be hematopoietic stem cells (HSC), HSC-derived endothelial cells, blood vascular endothelial cells, lymph vascular endothelial cells, endothelial cell lines, primary culture endothelial cells, endothelial cells derived from stem cell, bone marrow derived stem cell, cord blood derived cell, human umbilical vein endothelial cell (HUVEC), lymphatic endothelial cell, endothelial pregenitor cell, stem cell that differentiate into an endothelial cell, vascular progenitor cells from embryonic stem cells, endothelial cells from adipose tissue, or periodontal tissue or tooth pulp, preferably an HSC or an HSC-derived endothelial cell.

In various aspects, the matrix can comprise a material such as a fibrin, a fibrinogen, a collagen, a polyorthoester, a polyvinyl alcohol, a polyamide, a polycarbonate, ab agarose, an alginate, a poly(ethylene) glycol, a polylactic acid, a polyglycolic acid, a polycaprolactone, a polyvinyl pyrrolidone, a marine adhesive protein, a cyanoacrylate, a polymeric hydrogel, analogs, or a combination thereof. In some preferred configurations, the matrix material can be a polymeric hydrogel.

In various aspects, a matrix can include at least one macrochannel and/or microchannel. In some embodiments, a plurality of macrochannels can have an average diameter of at least about 0.1 mm up to about 50 mm. For example, macrochannels can have an average diameter of about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, about 10 mm, about 15 mm, about 20 mm, about 25 mm, about 30 mm, about 35 mm, about 40 mm, or about 45 mm.

In various aspects, a matrix can include at least one growth factor, preferably an angiogenic growth factor, more preferably bFGF, VEGF, PDGF, IGF, TGFb, or a combination thereof.

In various aspects, a tissue module of the present teachings can comprise tissue progenitor cells and/or vascular progenitor cells at a density of about 0.5 million total progenitor cells (M) ml⁻¹ to about 100 M ml⁻¹. For example, in various configurations, a tissue module can comprise progenitor cells at a density of about 1 M ml⁻¹, 5 M ml⁻¹, 10 M ml⁻¹, 15 M ml⁻¹, 20 M ml⁻¹, 25 M ml⁻¹, 30 M ml⁻¹, 35 M ml⁻¹, 40 M ml⁻¹, 45 M ml⁻¹, 50 M ml⁻¹, 55 M ml⁻¹, 60 M ml⁻¹, 65 M ml⁻¹, 70 M ml⁻¹, 75 M ml⁻¹, 80 M ml⁻¹, 85 M ml⁻¹, 90 M ml⁻¹, 95 M ml⁻¹, or 100 M ml⁻¹. In some configurations, a tissue module can comprise progenitor cells at a density of about 0.0001 million cells (M) ml⁻¹ to about 1000 M ml⁻¹. In some configurations, a tissue module can comprise progenitor cells at a density of at least about 1 M ml⁻¹ up to about 100 M ml⁻¹. In some configurations, a tissue module can comprise progenitor cells at a density of at least about 5 M ml⁻¹ up to about 95 M ml⁻¹. In some configurations, a tissue module can comprise progenitor cells at a density of at least about 10 M ml⁻¹ up to about 90 M ml⁻¹. In some configurations, a tissue module can comprise progenitor cells at a density of at least about 15 M ml⁻¹ up to about 85 M ml⁻¹. In some configurations, a tissue module can comprise progenitor cells at a density of at least about 20 M ml⁻¹ up to about 80 M ml⁻¹. In some configurations, a tissue module can comprise progenitor cells at a density of at least about 25 M ml⁻¹ up to about 75 M ml⁻¹. In some configurations, a tissue module can comprise progenitor cells at a density of at least about 30 M ml⁻¹ up to about 70 M ml⁻¹. In some configurations, a tissue module can comprise progenitor cells at a density of at least about 35 M ml⁻¹ up to about 65 M ml⁻¹. In some configurations, a tissue module can comprise progenitor cells at a density of at least about 40 M ml⁻¹ up to about 60 M ml⁻¹. In some configurations, a tissue module can comprise progenitor cells at a density of at least about 45 M ml⁻¹ up to about 55 M ml⁻¹. In some configurations, a tissue module can comprise progenitor cells at a density of at least about 45 M ml⁻¹ up to about 50 M ml⁻¹. In some configurations, a tissue module can comprise progenitor cells at a density of at least about 50 M ml⁻¹ up to about 55 M ml⁻¹.

In various aspects, the ratio of vascular progenitor cells to tissue progenitor cells can be from about 100:1 up to about 1:100. For example, the ratio of vascular progenitor cells to tissue progenitor cells can be about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. In some configurations, the ratio of vascular progenitor cells to tissue progenitor cells can be from about 20:1 up to about 1:20. In some configurations, the ratio of vascular progenitor cells to tissue progenitor cells can be from about 19:1 to about 1:19. In some configurations, the ratio of vascular progenitor cells to tissue progenitor cells can be from about 18:1 to about 1:18. In some configurations, the ratio of vascular progenitor cells to tissue progenitor cells can be from about in some configurations, the ratio of vascular progenitor cells to tissue progenitor cells can be from about 17:1 to about 1:17. In some configurations, the ratio of vascular progenitor cells to tissue progenitor cells can be from about in some configurations, the ratio of vascular progenitor cells to tissue progenitor cells can be from about 16:1 to about 1:16. In some configurations, the ratio of vascular progenitor cells to tissue progenitor cells can be from about in some configurations, the ratio of vascular progenitor cells to tissue progenitor cells can be from about 15:1 to about 1:15. In some configurations, the ratio of vascular progenitor cells to tissue progenitor cells can be from about 14:1 to about 1:14. In some configurations, the ratio of vascular progenitor cells to tissue progenitor cells can be from about 13:1 to about 1:13. In some configurations, the ratio of vascular progenitor cells to tissue progenitor cells can be from about 12:1 to about 1:12. In some configurations, the ratio of vascular progenitor cells to tissue progenitor cells can be from about 11:1 to about 1:11. In some configurations, the ratio of vascular progenitor cells to tissue progenitor cells can be from about 10:1 to about 1:10. In some configurations, the ratio of vascular progenitor cells to tissue progenitor cells can be from about 9:1 to about 1:9. In some configurations, the ratio of vascular progenitor cells to tissue progenitor cells can be from about in some configurations, the ratio of vascular progenitor cells to tissue progenitor cells can be from about 8:1 to about 1:8. In some configurations, the ratio of vascular progenitor cells to tissue progenitor cells can be from about 7:1 to about 1:7. In some configurations, the ratio of vascular progenitor cells to tissue progenitor cells can be from about 6:1 to about 1:6. In some configurations, the ratio of vascular progenitor cells to tissue progenitor cells can be from about 5:1 to about 1:5. In some configurations, the ratio of vascular progenitor cells to tissue progenitor cells can be from about 4:1 to about 1:4. In some configurations, the ratio of vascular progenitor cells to tissue progenitor cells can be from about in some configurations, the ratio of vascular progenitor cells to tissue progenitor cells can be from about 3:1 to about 1:3. In some configurations, the ratio of vascular progenitor cells to tissue progenitor cells can be from about in some configurations, the ratio of vascular progenitor cells to tissue progenitor cells can be from about 2:1 to about 1:2.

Yet another aspect of the invention provides methods of treating a tissue or organ defect. In various configurations, these methods include grafting a tissue module of the invention into a subject in need thereof.

A further aspect of the invention provides a method for identifying a candidate molecule that modulates tissue vascularization. Such methods include forming a tissue module of the present teachings; contacting the matrix, the tissue progenitor cells, the vascular progenitor cells, a combination thereof, or the tissue module with a candidate molecule; measuring vascularization of the engineered tissue composition; and determining whether the candidate molecule modulates blood vessel formation in the engineered tissue composition relative to a control not contacted with the candidate molecule. In some configurations, the candidate molecule can be contacted with the matrix, the tissue progenitor cells, or the vascular progenitor cells prior to combining a matrix with the progenitor cells, after cells have been seeded onto a matrix but before vascular morphogenesis have occurred, or after vascularization has commenced. As used herein, modulating tissue vascularization can include increasing vascularization or decreasing vascularization relative to a control.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a series of tissue section images depicting differentiation of human mesenchymal stem cells (hMSCs) into osteoblasts. FIG. 1A represents bone marrow sample prepared from one of multiple human donors showing abundant cells. FIG. 1B represents culture-expansion of hMSCs into spindle shaped cells from the population of adherent cells. FIG. 1C represents MSCs treated under osteogenic differentiation medium showing positive staining for alkaline phosphatase. FIG. 1D represents MSC-derived osteoblasts generating mineral nodules as revealed by von Kossa staining. Scale bar: 100 μm. Further details regarding methodology are presented in Example 1.

FIG. 2 is a series of images depicting engineered bone construct from both endothelial cells and osteoblasts derived from human mesenchymal stem cells (hMSCs). FIG. 2A represents osteoblasts derived from hMSCs seeded into the pores of tricalcium phosphate (TCP: light pink). Human umbilical vein endothelial cells (HUVEC) were expanded and seeded into a basement membrane hydrogel, Matrigel, in aqueous phase at 4 degrees Celsius, and infused into the pores of TCP, followed by gelation of Matrigel at 37 degrees Celsius. FIG. 2B demonstrates areas of bone-like tissue (B) among regions of TCP in samples retrieved after in vivo implantation in the dorsum of immunodeficient mice. FIG. 2C represents sections stained with H&E staining, which reveals the formation of lumens surrounded by round cells. Given that HUVECs were seeded homogenously in aqueous Matrigel, there was apparent reorganized of the seeded HUVECs in the formation of lumens and primitive vascular-like (PV) structures within the construct. FIG. 2D represents sections with Higher Magnification von Kossa staining, which reveals islands of mineralized tissue among TCP. Scale bar: 100 μm. Further details regarding methodology are presented in Example 1.

FIG. 3 is a series of images and a bar graph demonstrating differentiation of hematopoletic stem cells into endothelial cells towards engineering vascularized bone. FIG. 3A represents bone marrow isolated, CD34+, non-adherent cells plated on fibronectin-coated cell culture polystyrene. Although these cells were isolated form the same bone marrow as MSCs as shown in FIG. 1 above, the morphology of HSCs here is rounded, in sharp contracts to spindle shaped MSCs in FIG. 1B. FIG. 3B represents colony formation of HSCs following two weeks of culture. FIG. 3C demonstrates tubular structures formed between unconnected cells upon seeding colony-forming HSCs in Matrigel. FIG. 3D shows positive labeling to acetylated low density lipoproteins (Ac-LDLs) as evidenced by intracellular localization of Ac-LDLs fluorescence. FIG. 3E shows HSC-derived endothelial-like cells also expressed von Willebrand Factor (vWF), a marker for native endothelial cells: FIG. 3F demonstrates HSC-derived endothelial-like cells generated significantly more vWF (left bar) than control cells (fibroblasts) (right bar). Further details regarding methodology are presented in Example 2.

FIG. 4 is a series of cartoons depicting configurations of PEG hydrogel. FIG. 4A represents PEG hydrogel alone without either bFGF or macrochannels. FIG. 4B represents PEG hydrogel with 3 macrochannels created after photopolymerization (1 mm dia.), but without bFGF. FIG. 4C represents PEG hydrogel loaded with 10 ug/ml bFGF in solution followed by photopolymerization but without macro-channels. FIG. 4D represents PEG hydrogel with 10 ug/ml bFGF plus 3 macrochannels. From Stosich et al. (2006). Further details regarding methodology are presented in Example 3.

FIG. 5 is a series of photographic images depicting harvest of in vivo implanted samples. FIG. 5A shows harvested PEG hydrogel without cells, bFGF or channels showing a lack of macroscopic host tissue invasion. FIG. 5B shows harvested PEG hydrogel with 3 macrochannels (1 mm dia. Each) showing host tissue ingrowth in the lumen of engineered macrochannels. FIG. 5C shows harvested PEG hydrogel loaded with bFGF but without macrochannels showing general red color. FIG. 5D shows harvested PEG hydrogel with both bFGF and macrochannels showing general red color and host tissue ingrowth in the lumen of 3 engineered macrochannels. Scale bar: 6 mm. From Stosich et al. (2006). Further details regarding methodology are presented in Example 3.

FIG. 6 is a series of images depicting PEG hydrogel samples after 3-wk in vivo implantation, with H&E staining. FIG. 6A represents PEG hydrogel (H) without bFGF or macrochannels showed no host cell invasion. FIG. 6B represents host tissue ingrowth in PEG hydrogel (H) with 3 macrochannels (Carrow). Note the absence of host cell infiltration in the rest of PEG outside macrochannels. FIG. 6C depicts PEG hydrogel (H) loaded with bFGF but without channels showed apparently random host tissue infiltration. FIG. 6D represents host tissue infiltration; such infiltration took place only in macro-channels in bFGF-soaked PEG hydrogel. Despite the same bFGF dose in FIG. 6C and FIG. 6D, bFGF loaded PEG with macrochannels (FIG. 6D) induced substantial host tissue ingrowth. From Stosich et al. (2006). Further details regarding methodology are presented in Example 3.

FIG. 7 is a bar graph showing the amount of host tissue ingrowth by computerized histomorphometry. The amount of host tissue ingrowth in the macrochannels of PEG hydrogel loaded with bFGF is significantly greater than the amount of host tissue in macrochannels of PEG hydrogel without bFGF. N=8 per group. From Stosich et al., (2006). Further details regarding methodology are presented in Example 3.

FIG. 8 is series of images depicting H&E staining of ingrowing host tissue in PEG hydrogel. FIG. 8A represents PEG hydrogel with macrochannel but without bFGF showed host tissue ingrowth only in macrochannels. Arrow indicates a blood vessel. FIG. 8B represents higher power of FIG. 8A showing the blood vessel-like structure (white arrow) is lined by endothelial-like cells, and surrounded by fibroblast-like cells. FIG. 8C represents PEG hydrogel (H) loaded with bFGF but without macrochannels showed sparse ingrowth of host tissue and blood vessel-like structure lined by endothelial-like cells (black arrow). FIG. 8D represents higher power of FIG. 8C. FIG. 8E represents PEG hydrogel with both bFGF and macrochannels showing dense host tissue ingrowth in high density of blood vessel-like structures. (black arrow). FIG. 8F represents higher power image of FIG. 8E showing a large blood vessel-like structure (white arrow) with cells resembling red blood cells and lined by endothelial=like cells. Fibroblast-like cells surround the blood vessel-like structure. From Stosich et al., (2006). Further details regarding methodology are presented in Example 3.

FIG. 9 is a series of images depicting immunolocalized tissue sections with anti-VEGF antibody staining. FIG. 9A represents PEG hydrogel (H) without either bFGF or macrochannels showing a lack of VEGF positive tissue, except the host fibrous capsule (C). FIG. 9B represents PEG hydrogel with 3 macrochannels but without bFGF showing strong VEGF staining of the host tissue in macrochannels. FIG. 9C represents PEG hydrogel (H) loaded with bFGF but without macrochannels showing VEGF-positive tissue in an apparent random fashion. FIG. 9D represents PEG hydrogel (H) with both bFGF and macrochannels showing strong VEGF staining of host tissue in macrochannels. From Stosich et al. (2006). Further details regarding methodology are presented in Example 3.

FIG. 10 is a series of cartoons depicting experimental setup for cell density experiment. Human mesenchymal stem cells (MSCs), MSC=derived osteoblasts (MSC-Ob) and MSC-derived chondrocytes (MSC-Cy). For each cell lineage four cell densities were encapsulated in PEG hydrogel: 0, 5, 40 and 80 million cells per mL of cell suspension. OS medium: osteogenesis stimulating medium containing dexamethosone, ascorbic acid and b-glycerophosphate. CS medium chondrogenic medium containing TGFb3. FIG. 10A represents human MSCs without differentiation into any lineage. FIG. 10B represents human MSC-derived osteoblasts. FIG. 10C represents Human MSC-derived chondrocytes. In each case, cells cultured in 3D were tripsinized and loaded in cell suspension. The suspended cells were then loaded in the aqueous phase of PEG hydrogel, followed photo-polymerization and gelation. For each condition (A, B, and C), a gelated construct encapsulating MSCs, MSC-Ob and MSC-Cy is obtained for further in vitro and in vivo studies. From Troken and Mao (2006). Further details regarding methodology are presented in Example 4.

FIG. 11 is a series of images depicting histological observation of various cell densities after 4 week in vitro culture. Top row: Control or MSCs without differentiation cultured in DMEM. Middle row: MSC-osteoblasts (MSC-Ob) cultured in osteogenic medium. Bottom row: MSC-derived chondrocytes (MSC-Cy) cultured in chondrogenic medium. 5 M Cells/mL=5 millions cells per mL of cell suspension. The very left column represents cell-free PEG hydrogel. The next column represents an initial cell seeding density of 5 million cells per mL, followed by 40 million cells per mL and the very right column, 80 million cells per mL. For each cell lineage, initial cell seeding density was maintained upon 4 wk in vitro incubation. H&E staining. From Troken and Mao (2006). Further details regarding methodology are presented in Example 4.

FIG. 12 is a series of images depicting safranin O staining of PEG hydrogel encapsulating human mesenchymal stem cells (MSCs) (FIGS. 13A-13D) and MSC-derived chondrocytes (MSC-Cy) (FIGS. 13A′-13D′) after 4-wk in vitro culture. The very left column represents cell-free PEG hydrogel. The next column represents an initial cell encapsulation density of 5 million cells per mL, followed by 50 million cells per mL, and the very right column, 80 million cells per mL. Positive Safranin O staining shows labeling area as a function of the initial cell seeding density. MSCs were negative safranin O staining. The initial cell seeding density was maintained, along with the differentiated chondrogenic phenotype in PEG hydrogel. From Troken and Mao (2006). Further details regarding methodology are presented in Example 4.

FIG. 13 is a series of images depicting Von Kossa staining of PEG hydrogel encapsulating human mesenchymal stem cells (MSCs) (FIGS. 14A-14D) and MSC-derived osteoblasts (MSC-Ob) (FIGS. 14A′-14D′) after 4-wk in vitro culture. The very left column represents cell-free PEG hydrogel. The next column represents an initial cell encapsulation density of 5 millions cells per mL, followed by 40 millions cells per mL and the very right column, 80 million cells per mL. Von Kossa is positive and shows labeling area as a function of the initial cell seeding density. MSCs were negative von Kossa staining. This suggests that MSCs have not differentiated into osteoblasts without addition of osteogenic stimulants as in the lower row. The initial cell encapsulation densities were maintained, along with the differentiated osteogenic phenotype in PEG hydrogel. From Troken and Mao (2006). Further details regarding methodology are presented in Example 4.

FIG. 14 is a pair of bar graphs showing quantification of matrix formation of MSC-derived chondrocytes and MSC-derived osteoblasts. FIG. 14A represents total Alcian blue area over total scaffold area following 4-wk in vivo implantation. MSC-derived chondrocytes (MSC-Cy) synthesized significantly more GAG than hMSCs and HMSC-derived osteoblasts (hMSC-Ob). FIG. 14B represents total von Kossa area over total scaffold area. MSC-Ob induced significantly more mineralization than hMSCs and HMSC-Cy. N=8 per group. From Troken and Mao (2006). Further details regarding methodology are presented in Example 4.

FIG. 15 is a series of cartoons depicting configurations of PEG hydrogel and the corresponding immunohistochemistal image of the implanted hydrogel after 4 weeks. FIG. 15A depicts a PEG hydrogel with macrochannels but no bFGF. FIG. 15B depicts a PEG hydrogel with bFGF and no macrochannels. FIG. 15C depicts a PEG hydrogel with macrochannels and bFGF. FIG. 15A′ is an immunohistochemistal tissue image of the implanted PEG hydrogel of FIG. 15A. FIG. 15B′ is an immunohistochemistal tissue image of the implanted PEG hydrogel of FIG. 15B. FIG. 150′ is an immunohistochemistal tissue image of the implanted PEG hydrogel of FIG. 150. Further details regarding methodology are presented in Example 20.

FIG. 16 is a series of images depicting human mesenchymal cells differentiated into adipogenic cells in vitro over 35 days in ex vivo culture. Sections are stained with Oil-red O, to which hMSC derived adipogenic cells react positively. FIGS. 16A-16E represent hMSCs without adipogenic differentiation, while FIGS. 16F-16J represent hMSC derived adipogenic cells. Further details regarding methodology are presented in Examples 21-22.

FIG. 17 is a series of bar graphs the total DNA content of culture samples between hMSCs and hMSC-derived adipogenic cells over 35 days (FIG. 17A) and glycerol contents of hMSCs and hMSC-derived adipogenic cell samples (FIG. 17B). Further details regarding methodology are presented in Example 22.

FIG. 18 is a series of cartoons and photographic images depicting vascularized adipogenesis of hMSCs and hMSC-derived adipogenic cells encapsulated in PEG hydrogel after implantation for four weeks. FIG. 18A depicts a PEG hydrogel with no macrochannels, no bFGF, and no cells delivered. FIG. 18B depicts a PEG hydrogel with macrochannels, with bFGF, and with no cells delivered. FIG. 18C depicts a PEG hydrogel with macrochannels, with bFGF, and with hMSC-adipocytes delivered. FIGS. 19A′, 19B′. and 19C′ are photographic images of the PEG hydrogels of FIGS. 19A, 19B, and 19C, respectively, after implanted for twelve weeks in mice. Further details regarding methodology are presented in Example 23.

FIG. 19 is a series of images depicting stained tissue sections of tissue with PEG microchanneled hydrogel with macrochannels encapsulating hMSC-derived adipogenic cells implanted for twelve weeks. FIG. 19A is immunohistochemical stained tissue. FIG. 19B is tissue stained with Oil-red O positive. FIG. 19C is tissue stained with Anti-VEGF antibody. FIG. 19D is tissue stained with anti-WGA lectin antibody. Further details regarding methodology are presented in Example 23.

FIG. 20 is a series of images depicting vascular endothelial growth factors 2 or Flk1 expression in vascular progenitor cells. Further details regarding methodology are presented in Example 24.

FIG. 21 is a bar graph showing quantification of VEGF2 in vascular progenitor cells. Further details regarding methodology are presented in Example 24.

FIG. 22 is an image depicting osteoprogenitors labeled with green fluorescence protein (GFP) and vascular progenitor cells labeled with CM-DII in red in a porous βTCP scaffold. Further details regarding methodology are presented in Example 25.

### DETAILED DESCRIPTION OF THE INVENTION

The approaches described herein are based at least in part upon application of the discovery of vascularized tissue formation by combined actions of hematopoietic and mesenchymal stem cells to tissue engineering. Demonstrated herein is the vascularization of polymeric biomaterials when combined with tissue progenitor cells and vascular progenitor cells. Also demonstrated is that vascular progenitor cells, when introduced into or onto a porous scaffold containing tissue progenitor cells, induce blood vessel-like structures in vivo. Further demonstrated is that physically built-in macrochannels and/or an angiogenic growth factor in a matrix material induce host-derived angiogenesis and vascularization in vivo.

Thus is provided a novel regenerative approach for tissue defects from synergistic actions of both vascular progenitor cells and tissue progenitor cells, such that the total effect can be greater than the sum of the individual effects. Such approaches benefit from the new understanding, disclosed herein, of the interactions between vascular progenitor cells (e.g., HSCs), tissue progenitor cells (e.g., MSCs), and their cell lineage derivatives with regulatory angiogenic growth factors in the de novo formation of vascularized tissues or organs. As an example, the compositions and methods described herein can provide biologically viable engineered hard tissue modules for the repair of long-bone defects such as segmental defects, subchondral bone regeneration in biologically derived total joint replacement, and bone marrow replacement. As another example, the compositions and methods described herein can provide biologically viable engineered soft adipose tissue modules for the repair of soft tissue defects resulting from trauma, tumor resection, and congenital anomalies.

One aspect of the invention provides for compositions of engineered vascularized tissue or organ. Such compositions generally include tissue progenitor cells and vascular progenitor cells introduced into or onto a biocompatible matrix. Another aspect of the invention provides methods for the formation of such engineered vascularized tissue or organ. According to these methods for tissue engineering and tissue regeneration, tissue progenitor cells and vascular progenitor cells are introduced into or onto a biocompatible matrix so as to produce a vascularized tissue or organ. A further aspect provides a method of treating a tissue defect by grafting a composition of the invention into a subject in need thereof.

Biologically viable tissue or organ can be engineered from tissue progenitor cells with improved vascularization through the use of vascular progenitor cells. Vascularized tissue or organ types that can be formed according to the methods described herein include, but are not limited to, bladder, bone, brain, breast, osteochondral junction, nervous tissue including central nerveous system, spinal cord and peripheral nerve, glia, esophagus, fallopian tube, heart, pancreas, intestines, gallbladder, kidney, liver, lung, ovaries, prostate, spinal cord, spleen, skeletal muscle, skin, stomach, testes, thymus, thyroid, trachea, urogenital tract, ureter, urethra, interstitial soft tissue, periosteum, periodontal tissue, cranial sutures, hair follicles, oral mucosa, and uterus. A preferable soft tissue composition formed by methods of the invention is engineered vascularized adipose tissue. A preferable hard tissue composition formed by methods of the invention is engineered vascularized bone tissue.

A tissue is generally a collection of cells having a similar morphology and function, and frequently supported by heterogenous interstitial tissues with multiple cell types and blood supply. An organ is generally a collection of tissues that perform a biological function. Organs can be, but are not limited to, bladder, brain, nervous tissue, glial tissue, esophagus, fallopian tube, bone, synovial joint, cranial sutures, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage. The biological function of an organ can be assayed using standard methods known to the skilled artisan.

Infusion and Culturing

To form the compositions of the invention, tissue progenitor cells and vascular progenitor cells are introduced (e.g., implanted, injected, infused, or seeded) into or onto an artificial structure (e.g., a scaffold comprising a matrix material) capable of supporting three-dimensional tissue or organ formation. The tissue progenitor cells and vascular progenitor cells can be co-introduced or sequentially introduced. The tissue progenitor cells and vascular progenitor cells can be introduced in the same spatial position, similar spatial positions, or different spatial positions, relative to each other. Preferably, tissue progenitor cells and vascular progenitor cells introduced into or onto different areas of the matrix material. It is contemplated that more than one type of tissue progenitor cell can be introduced into the matrix. Similarly, it is contemplated that more than one type of vascular progenitor cell can be introduced into the matrix.

Tissue progenitor cells and/or vascular progenitor cells can be introduced into the matrix material by a variety of means known to the art (see e.g., Example 1; Example 4; Example 11; Example 12; Example 20, Example 23). Methods for the introduction (e.g., infusion, seeding, injection, etc.) of progenitor cells into or into the matrix material are discussed in, for example, Ma and Elisseeff, ed. (2005) Scaffolding In Tissue Engineering, CRC, ISBN 1574445219; Saltzman (2004) Tissue Engineering: Engineering Principles for the Design of Replacement Organs and Tissues, Oxford ISBN. 019514130X; Minuth et al. (2005) Tissue Engineering: From Cell Biology to Artificial Organs, John Wiley & Sons, ISBN 3527311866. For example, progenitor cells can be introduced into or onto the matrix by methods including hydrating freeze-dried scaffolds with a cell suspension (e.g., at a concentration of 100 cells/ml to several million cells/ml). Methods of addition of additional agents vary, as discussed below.

Methods of culturing and differentiating progenitor cells in or on scaffolds are generally known in the art (see e.g., Saltzman (2004) Tissue Engineering: Engineering Principles for the Design of Replacement Organs and Tissues, Oxford ISBN 019514130X; Vunjak-Novakovic and Freshney, eds. (2006) Culture of Cells for Tissue Engineering, Wiley-Liss, ISBN 0471629359; Minuth et al. (2005) Tissue Engineering: From Cell Biology to Artificial Organs, John Wiley & Sons, ISBN 3527311866). As will be appreciated by one skilled in the art, the time between progenitor cell introduction into or onto the matrix and engrafting the resulting matrix can vary according to particular application. Incubation (and subsequent replication and/or differentiation) of the engineered composition containing tissue progentior cells and vascular progenitor cells in or on the matrix material can be, for example, at least in part in vitro, substantially in vitro, at least in part in vivo, or substantially in vivo. Determination of optimal culture time is within the skill of the art. A suitable medium can be used for in vitro progenitor cell infusion, differentiation, or cell transdifferentiation (see e.g., Vunjak-Novakovic and Freshney, eds. (2006) Culture of Cells for Tissue Engineering, Wiley-Liss, ISBN 0471629359; Minuth et al. (2005) Tissue Engineering: From Cell Biology to Artificial Organs, John Wiley & Sons, ISBN 3527311866). The culture time can vary from about an hour, several hours, a day, several days, a week, or several weeks. The quantity and type of cells present in the matrix can be characterized by, for example, morphology by ELISA, by protein assays, by genetic assays, by mechanical analysis, by RT-PCR, and/or by immunostaining to screen for cell-type-specific markers (see e.g., Minuth et al., (2005) Tissue Engineering: From Cell Biology to Artificial Organs, John Wiley & Sons, ISBN 3527311866).

In some embodiments, the engineered vascularized tissue or organ composition is formed by introducing tissue progenitor cells and vascular progenitor cells into or onto a matrix material, as described herein, without requiring the use of additional biologically active agents, especially growth factors and the like. The ability to form engineered vascularized tissue or organ in the absence of growth factors provides an advantage in tissue engineering not reflected by conventional processes.

Vascularization

The introduction of tissue progenitor cells and vascular progenitor cells into or onto the matrix material occurs under conditions that result in the vascularization of the composition. Preferably, the blood vessels grow throughout the engineered tissue or organ. Vascularization can be produced in the engineered tissue or organ in vitro (see e.g., Example 2; Example 22), in vivo (see e.g., Example 1; Example 23), or a combination thereof. For example, differentiation can be carried out by culturing tissue progenitor cells and vascular progenitor cells in the matrix material of the scaffold. As another example, the progenitor cells can be infused into the matrix, and such matrix promptly engrafted into a subject, allowing differentiation to occur in vivo. The determination of when to introduce the engineered tissue or organ into a subject can be based, at least in part, on the amount of vascularization formed in the tissue or organ.

Methods for measuring angiogenesis in the engineered tissue or organ are standard in the art (see e.g., Jain et al. (2002) Nat. Rev. Cancer 2:266-276; Ferrara, ed. (2006) Angiogenesis, CRC, ISBN 0849328446). During early blood vessel formation, immature vessels resemble the vascular plexus during development, by having relatively large diameters and lacking morphological vessel differentiation. Over time, the mesh-like pattern of immature angiogenic vessels gradually mature into functional microcirculatory units, which develop into a dense capillary network having differentiated arterioles and venules. Angiogenesis can be assayed, for example, by measuring the number of non-branching blood vessel segments (number of segments per unit area), the functional vascular density (total length of perfused blood vessel per unit area), the vessel diameter, or the vessel volume density (total of calculated blood vessel volume based on length and diameter of each segment per unit area).

The compositions of the invention generally have increased vascularization as compared to engineered tissue or organ produced according to conventional means. For example, blood vessel formation (e.g., angiogenesis, vasculogenesis, formation of an immature blood vessel network, blood vessel remodeling, blood vessel stabilization, blood vessel maturation, blood vessel differentiation, or establishment of a functional blood vessel network) in the engineered tissue or organ can be increased by at least 5%, 10%, 20%, 25%, 30%, 40%, or 50%, 60%, 70%, 80%, 90%, or even by as much as 100%, 150%, or 200% compared to a corresponding engineered tissue or organ that is not formed by introducing both vascular progenitor cells and tissue progenitor cells as descried herein. The vascularization of the engineered tissue or organ composition is preferably a stable network of blood vessels that endures for at least 1 day, 2 days, 3 days, 4 days, 5 days, 6 days, 1 week, 2 weeks, 3 weeks, 1 month, 2 months, 3 months, 4 months, 5 months, 6 months, or even 12 months or more. Preferably, the vascular network of the engineered tissue or organ composition in integrated into the circulatory system of the tissue, organ, or subject upon introduction thereto.

For tissue or organ regeneration using small scaffolds (<100 cubic millimeters in size), in vitro medium can be changed manually, and additional agents added periodically (e.g., every 3-4 days). For larger scaffolds, the culture can be maintained, for example, in a bioreactor system, which may use a minipump for medium change. The minipump can be housed in an incubator, with fresh medium pumped to the matrix material of the scaffold. The medium circulated back to, and through, the matrix can have about 1% to about 100% fresh medium. The pump rate can be adjusted for optimal distribution of medium and/or additional agents included in the medium. The medium delivery system can be tailored to the type of tissue or organ being manufactured. All culturing is preferably performed under sterile conditions.

Progenitor Cells

Compositions and methods of the invention employ both tissue progenitor cells and vascular progenitor cells. Such cells can be isolated, purified, and/or cultured by a variety of means known to the art (see e.g., Example 9; Example 21). Methods for the isolation and culture of progenitor cells are discussed in, for example, Vunjak-Novakovic and Freshney (2006) Culture of Cells for Tissue Engineering, Wiley-Liss, ISBN 0471629359. In some aspects, progenitor cells can be derived from the same or different species as an intended transplant recipient. For example, progenitor cells can be derived from an animal, including, but not limited to, a vertebrate such as a mammal, a reptile, or an avian. In some configurations, a mammal or avian is preferably a horse, a cow, a dog, a cat, a sheep, a pig, or a chicken, and most preferably a human.

Tissue progenitor cells of the present teachings include cells capable of differentiating into a target tissue or organ, and/or undergoing morphogenesis to form the target tissue or organ. Non-limiting examples of tissue progenitor cells include mesenchymal stem cells (MSCs), cells differentiated from MSCs, osteoblasts, chondrocytes, myocytes, adipocytes, neuronal cells, neuronal supporting cells such as neural glial cells (such as Schwann cells), fibroblastic cells such as interstitial fibroblasts, tendon fibroblasts, dermal fibroblasts, ligament fibroblasts, periodontal fibroblasts such as gingival fibroblasts, craniofacial fibroblasts, cardiomyocytes, epithelial cells, liver cells, uretheral cells, kidney cells, periosteal cells, bladder cells, beta-pancreatic islet cell, odontoblasts, dental pulp cells, periodontal cells, lung cells, and cardiac cells. For example, in vascularized bone tissue of the invention, tissue progenitor cells introduced into a matrix can be progenitor cells that can give rise to bone tissue such as mesenchymal stem cells (MSC), MSC osteoblasts, or MSC chondrocytes. It is understood that MSC chondrocytes are chondrocytes differentiated from MSCs. Similarly, MSC osteoblasts are osteoblasts MSC osteoblasts. In another example, in vascularized adipose tissue of the invention, tissue progenitor cells introduced into a matrix can be progenitor cells that can give rise to adipose tissue, such as MSCs or MSC adipogenic cells (i.e., adipogenic cells differentiated from MSCs).

Vascular progenitor cells introduced into or onto the matrix material are progenitor cells capable of differentiating into or otherwise forming vascular tissue. Vascular progenitor cells can be, for example, stem cells that can differentiate into endothelial cells such as hematopoietic stem cells (HSC), HSC endothelial cells, blood vascular endothelial cells, lymph vascular endothelial cells, endothelial cell lines, primary culture endothelial cells, endothelial cells derived from stem cells, bone marrow derived stem cells, cord blood derived cells, human umbilical vein endothelial cells (HUVEC), lymphatic endothelial cells, endothelial progenitor cells, endothelial cell lines, endothelial cells generated from stem cells in vitro, endothelial cells extracted from adipose tissue, smooth muscle cells, interstitial fibroblasts, myofibroblasts, periodontal tissue, tooth pulp, or vascular-derived cells. It is understood that HSC endothelial cells are endothelial cells differentiated from HSCs. Vascular progenitor cells can be isolated from, for example, bone marrow, soft tissue, muscle, tooth, blood and/or vascular system. In some configurations, vascular progenitor cells can be derived from tissue progenitor cells.

The present teachings include methods for optimizing the density of both tissue progenitor cells and vascular progenitor cells, (and their lineage derivatives) so as to maximize the regenerative outcome of a vascularized tissue or organ (see e.g., Example 4; Example 5; Example 6). In these methods, cell densities in a matrix can be monitored over time and at end-points. Tissue properties can be determined, for example, using standard techniques known to skilled artisans, such as histology, structural analysis, immunohistochemistry, biochemical analysis, and mechanical properties. As will be recognized by one skilled in the art, the cell densities of tissue progenitor cells and/or vascular progenitor cells can vary according to, for example, progenitor type, tissue or organ type, matrix material, matrix volume, infusion method, seeding pattern, culture medium, growth factors, incubation time, incubation conditions, and the like. Generally, for both the tissue progenitor cells and the vascular progenitor cells, the cell density of each cell type in a matrix can be, independently, from 0.0001 million cells (M) ml⁻¹ to about 1000 M ml⁻¹. For example, the tissue progenitor cells and the vascular progenitor cells can each be present in the matrix at a density of about 0.001 M ml⁻¹, 0.01 M ml⁻¹, 0.1 M ml⁻¹, 1 M ml⁻¹, 5 M ml⁻¹, 10 M ml⁻¹, 15 M ml⁻¹, 20 M ml⁻¹, 25 M ml⁻¹, 30 M ml⁻¹, 35 M ml⁻¹, 40 M ml⁻¹, 45 M ml⁻¹, 50 M ml⁻¹, 55 M ml⁻¹, 60 M ml⁻¹, 65 M ml⁻¹, 70 M ml⁻¹, 75 M ml⁻¹, 80 M ml⁻¹, 85 M ml⁻¹, 90 M ml⁻¹, 95 M ml⁻¹, 100 M ml⁻¹, 200 M ml⁻¹, 300 M ml⁻¹, 400 M ml⁻¹, 500 M ml⁻¹, 600 M ml⁻¹, 700 M ml⁻¹, 800 M ml⁻¹, or 900 M ml⁻¹.

Vascular progenitor cells and tissue progenitor cells can be introduced at various ratios in or on the matrix (see Example 5). As will be recognized by one skilled in the art, the cell ratio of vascular progenitor cells to tissue progenitor cells can vary according to, for example, type of progenitor cells, target tissue or organ type, matrix material, matrix volume, infusion method, seeding pattern, culture medium, growth factors, incubation time, and/or incubation conditions. Generally, the ratio of vascular progenitor cells to tissue progenitor cells can be about 100:1 to about 1:100. For example, the ratio of vascular progenitor cells to tissue progenitor cells can be about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20.

In some embodiments, the progenitor cells introduced to the matrix can comprise a heterologous nucleic acid so as to express a bioactive molecule such as heterologous protein, or to overexpress an endogenous protein. In non-limiting example, progenitor cells introduced to the matrix can express a fluorescent protein marker, such as GFP, EGFP, BFP, CFP, YFP, or RFP. In another example, progenitor cells introduced to the matrix can express an angiogenesis-related factor, such as activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin and fibronectin receptor α₅β₁, Factor X, HB-EGF, HBNF, HGF, HUAF, heart derived inhibitor of vascular cell proliferation, IFN-gamma, IL1, IGF-2 IFN-gamma, integrin receptors (e.g., various combinations of α subunits (e.g., α₁, α₂, α₃, α₄, α₅, α₆, α₇, α₈, α₉, α_{E}, α_{V}, α_{IIb}, α_{L}, α_{M}, α_{X}), K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-beta, Tie 1, Tie2, TGF-β, and TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF.sub.164, VEGI, EG-VEGF, VEGF receptors, PF4, 16 kDa fragment of prolactin, prostaglandins E1 and E2, steroids, heparin, 1-butyryl glycerol (monobutyrin), or nicotinic amide. As another example, progenitor cells introduced to a matrix can comprise genetic sequences that reduce or eliminate an immune response in the host (e.g., by suppressing expression of cell surface antigens such as class I and class II histocompatibility antigen).

In some embodiments, one or more cell types in addition to a first tissue progenitor cell and a first vascular progenitor cell can be introduced into or onto the matrix material. Such additional cell type can be selected from those discussed above, and/or can include (but not limited to) skin cells, liver cells, heart cells, kidney cells, pancreatic cells, lung cells, bladder cells, stomach cells, intestinal cells, cells of the urogenital tract, breast cells, skeletal muscle cells, skin cells, bone cells, cartilage cells, keratinocytes, hepatocytes, gastro-intestinal cells, epithelial cells, endothelial cells, mammary cells, skeletal muscle cells, smooth muscle cells, parenchymal cells, osteoclasts, or chondrocytes. These cell-types can be introduced prior to, during, or after vascularization of the matrix. Such introduction may take place in vitro or in vivo. When the cells are introduced in vivo, the introduction may be at the site of the engineered vascularized tissue or organ composition or at a site removed therefrom. Exemplary routes of administration of the cells include injection and surgical implantation.

Matrix

The compositions and methods of the invention employ a matrix, into or onto which progenitor cells are introduced so as to form a vascularized tissue or organ construct. Such matrix materials can: allow cell attachment and migration; deliver and retain cells and biochemical factors; enable diffusion of cell nutrients and expressed products; and/or exert certain mechanical and biological influences to modify the behavior of the cell phase. The matrix is generally a porous, microcellular scaffold of a biocompatible material that provides a physical support and an adhesive substrate for introducing vascular progenitor cells and tissue progenitor cells during in vitro culturing and subsequent in vivo implantation. A matrix with a high porosity and an adequate pore size is preferred so as to facilitate cell introduction and diffusion throughout the whole structure of both cells and nutrients. Matrix biodegradability is also preferred since absorption of the matrix by the surrounding tissues can eliminate the necessity of a surgical removal. The rate at which degradation occurs should coincide as much as possible with the rate of tissue or organ formation. Thus, while cells are fabricating their own natural structure around themselves, the matrix is able to provide structural integrity and eventually break down leaving the neotissue, newly formed tissue or organ which can assume the mechanical load. Injectability is also preferred in some clinical applications. Suitable matrix materials are discussed in, for example, Ma and Elisseeff, ed. (2005) Scaffolding in Tissue Engineering, CRC, ISBN 1574445219; Saltzman (2004) Tissue Engineering: Engineering Principles for the Design of Replacement Organs and Tissues, Oxford ISBN 019514130X.

The matrix configuration can be dependent on the tissue or organ that is to be repaired or produced, but preferably the matrix is a pliable, biocompatible, porous template that allows for vascular and target tissue or organ growth. The matrix can be fabricated into structural supports, where the geometry of the structure (e.g., shape, size, porosity, micro- or macro-channels) is tailored to the application. The porosity of the matrix is a design parameter that influences cell introduction and/or cell infiltration. The matrix can be designed to incorporate extracellular matrix proteins that influence cell adhesion and migration in the matrix.

The matrix can be formed of synthetic polymers. Such synthetic polymers include, but are not limited to, polyurethanes, polyorthoesters, polyvinyl alcohol, polyamides, polycarbonates, poly(ethylene) glycol, polylactic acid, polyglycolic acid, polycaprolactone, polyvinyl pyrrolidone, marine adhesive proteins, and cyanoacrylates, or analogs, mixtures, combinations, and derivatives of the above.

Alternatively, the matrix can be formed of naturally occurring polymers or natively derived polymers. Such polymers include, but are not limited to, agarose, alginate, fibrin, fibrinogen, fibronectin, collagen, gelatin, hyaluronic acid, and other suitable polymers and biopolymers, or analogs, mixtures, combinations, and derivatives of the above. Also, the matrix can be formed from a mixture of naturally occurring biopolymers and synthetic polymers.

The matrix material the matrix can include, for example, a collagen gel, a polyvinyl alcohol sponge, a poly(D,L-lactide-co-glycolide) fiber matrix, a polyglactin fiber, a calcium alginate gel, a polyglycolic acid mesh, polyester (e.g., poly-(L-lactic acid) or a polyanhydride), a polysaccharide (e.g. alginate), polyphosphazene, or polyacrylate, or a polyethylene oxide-polypropylene glycol block copolymer. Matrices can be produced from proteins (e.g. extracellular matrix proteins such as fibrin, collagen, and fibronectin), polymers (e.g., polyvinylpyrrolidone), or hyaluronic acid. Synthetic polymers can also be used, including bioerodible polymers (e.g., poly(lactide), poly(glycolic acid), poly(lactide-co-glycolide), poly(caprolactone), polycarbonates, polyamides, polyanhydrides, polyamino acids, polyortho esters, polyacetals, polycyanoacrylates), degradable polyurethanes, non-erodible polymers (e.g., polyacrylates, ethylene-vinyl acetate polymers and other acyl substituted cellulose acetates and derivatives thereof), non-erodible polyurethanes, polystyrenes, polyvinyl chloride, polyvinyl fluoride, poly(vinylimidazole), chlorosulphonated polyolifins, polyethylene oxide, polyvinyl alcohol, Teflon®, and nylon.

The matrix can also include one or more of enzymes, ions, growth factors, and/or biologic agents. For example, the matrix can contain a growth factor (e.g., and angiogenic growth factor, or tissue specific growth factor). Such a growth factor can be supplied at a concentration of about 0 to 1000 ng/mL. For example, the growth factor can be present at a concentration of about 100 to 700 ng/mL, at a concentration of about 200 to 400 ng/mL, or at a concentration of about 250 ng/mL.

The matrix can contain one or more physical channels. Such physical channels include microchannels and macrochannels. Microchannels generally have an average diameter of about 0.1 μm to about 1,000 μm. As shown herein, matrix macrochannels can accelerate angiogenesis and bone or adipose tissue formation, as well as direct the development of vascularization and host cell invasion (see e.g., Example 3; Example 20; Example 23). Microchannels and/or macrochannels can be a naturally occurring feature of certain matrix materials and/or specifically engineered in the matrix material. Formation of microchannels and/or macrochannels can be according to, for example, mechanical and/or chemical means.

Macrochannels can extend variable depths through the matrix, or completely through the matrix. Macrochannels can be a variety of diameters. Generally, the diameter of the macrochannel can be chosen according to increased optimization of perfusion, tissue growth, and vascularization of the tissue module. The macrochannels can have an average diameter of, for example, about 0.1 mm to about 50 mm. For example, macrochannels can have an average diameter of about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, about 10 mm, about 15 mm, about 20 mm, about 25 mm, about 30 mm, about 35 mm, about 40 mm, or about 45 mm.

On skilled in the art will understand that the distribution of macrochannel diameters can be a normal distribution of diameters or a non-normal distribution diameters.

Added Drugs and/or Diagnostics

In some embodiments, the methods and compositions of the invention further comprise additional agents introduced into or onto the matrix along with the tissue progenitor cells and the vascular progenitor cells. Various agents that can be introduced include, but are not limited to, bioactive molecules, biologic drugs, diagnostic agents, and strengthening agents.

The matrix can further comprise a bioactive molecule. The cells of the matrix can be, for example, genetically engineered to express the bioactive molecule or the bioactive molecule can be added to the matrix. The matrix can also be cultured in the presence of the bioactive molecule. The bioactive molecule can be added prior to, during, or after progenitor cells are introduced to the matrix. Non-limiting examples of bioactive molecules include activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin, fibronectin receptor α₅β₁, Factor X, HB-EGF, HBNF, HGF, HUAF, heart derived inhibitor of vascular cell proliferation, IFN-gamma, IL1, IGF-2 IFN-gamma, integrin receptors (e.g., various combinations of α subunits (e.g., α₁, α₂, α₃, α₄, α₅, α₆, α₇, α₈, α₉, α_{E}, α_{V}, α_{IIb}, α_{L}, α_{M}, α_{X}) and β subunits (e.g., β₁, β₂, β₃, β₄, β₅, β₆, β₇, and β₈)), K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPARV ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-β, Tie 1, Tie2, TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF₁₆₄, VEGI, EG-VEGF, VEGF receptors, PF4, 16 kDa fragment of prolactin, prostaglandins E1 and E2, steroids, heparin, 1-butyryl glycerol (monobutyrin), and nicotinic amide. In other preferred embodiments, the matrix includes a chemotherapeutic agent or immunomodulatory molecule. Such agents and molecules are known to the skilled artisan. Preferably, the matrix includes bFGF, VEGF, or PDGF, or some combination thereof (see Example 3; Example 7).

Regulation of HSC- and MSC-derived angiogenesis in engineered tissue grafts can be according to controlled release of growth factors. Engineered blood vessels can be “leaky” as a result of abnormally high permeability of endothelial cells. Maturation of human HSC endothelial cells can be enhanced by micro-encapsulated delivery of angiogenic growth factors in HSC- and MSC-derived vascularized tissue grafts implanted in vivo.

Biologic drugs that can be added to the compositions of the invention include immunomodulators and other biological response modifiers. A biological response modifier generally encompasses a biomolecule (e.g., peptide, peptide fragment, polysaccharide, lipid, antibody) that is involved in modifying a biological response, such as the immune response or tissue or organ growth and repair, in a manner which enhances a particular desired therapeutic effect, for example, the cytolysis of bacterial cells or the growth of tissue- or organ-specific cells or vascularization. Biologic drugs can also be incorporated directly into the matrix component. Those of skill in the art will know, or can readily ascertain, other substances which can act as suitable non-biologic and biologic drugs.

Compositions of the invention can also be modified to incorporate a diagnostic agent, such as a radiopaque agent. The presence of such agents can allow the physician to monitor the progression of wound healing occurring internally. Such compounds include barium sulfate as well as various organic compounds containing iodine. Examples of these latter compounds include iocetamic acid, iodipamide, iodoxamate meglumine, iopanoic acid, as well as diatrizoate derivatives, such as diatrizoate sodium. Other contrast agents which can be utilized in the compositions of the invention can be readily ascertained by those of skill in the art and may include the use of radiolabeled fatty acids or analogs thereof.

The concentration of agent in the composition will vary with the nature of the compound, its physiological role, and desired therapeutic or diagnostic effect. A therapeutically effective amount is generally a sufficient concentration of therapeutic agent to display the desired effect without undue toxicity. A diagnostically effective amount is generally a concentration of diagnostic agent which is effective in allowing the monitoring of the integration of the tissue graft, while minimizing potential toxicity. In any event, the desired concentration in a particular instance for a particular compound is readily ascertainable by one of skill in the art.

The matrix composition can be enhanced, or strengthened, through the use of such supplements as human serum albumin (HSA), hydroxyethyl starch, dextran, or combinations thereof. The solubility of the matrix compositions can also be enhanced by the addition of a nondenaturing nonionic detergent, such as polysorbate 80. Suitable concentrations of these compounds for use in the compositions of the invention will be known to those of skill in the art, or can be readily ascertained without undue experimentation. The matrix compositions can also be further enhanced by the use of optional stabilizers or diluent. The proper use of these would be known to one of skill in the art, or can be readily ascertained without undue experimentation.

Implanting

The engineered tissue or organ compositions of the invention hold significant clinical value because of their increased levels of vascularization, as compared to other engineered tissues or organs of similar stages produced by other means known to the art. It is this increase in vascularization, enabling more efficient regeneration of tissue and organ, which sets the compositions of the invention apart from other conventional treatment options.

A determination of the need for treatment will typically be assessed by a history and physical exam consistent with the tissue or organ defect at issue. Subjects with an identified need of therapy include those with a diagnosed tissue or organ defect. The subject is preferably an animal, including, but not limited to, mammals, reptiles, and avians, more preferably horses, cows, dogs, cats, sheep, pigs, and chickens, and most preferably human.

As an example, a subject in need may have a deficiency of at least 5%, 10%, 25%, 50%, 75%, 90% or more of a particular cell type. As another example, a subject in need may have damage to a tissue or organ, and the method provides an increase in biological function of the tissue or organ by at least 5%, 10%, 25%, 50%, 75%, 90%, 100%, or 200%, or even by as much as 300%, 400%, or 500%. As yet another example, the subject in need may have a disease, disorder, or condition, and the method provides an engineered tissue or organ construct sufficient to ameliorate or stabilize the disease, disorder, or condition. For example, the subject may have a disease, disorder, or condition that results in the loss, atrophy, dysfunction, or death of cells. Exemplary treated conditions include a neural, glial, or muscle degenerative disorder, muscular atrophy or dystrophy, heart disease such as congenital heart failure, hepatitis or cirrhosis of the liver, an autoimmune disorder, diabetes, cancer, a congenital defect that results in the absence of a tissue or organ, or a disease, disorder, or condition that requires the removal of a tissue or organ, ischemic diseases such as angina pectoris, myocardial infarction and ischemic limb, accidental tissue defect or damage such as fracture or wound. In a further example, the subject in need may have an increased risk of developing a disease, disorder, or condition that is delayed or prevented by the method.

The tissue or organ can be selected from bladder, brain, nervous tissue, glia, esophagus, fallopian tube, heart, pancreas, intestines, gall bladder, kidney, liver, lung, ovaries, prostate, spinal cord, spleen, stomach, testes, thymus, thyroid, trachea, urogenital tract, ureter, urethra, uterus, breast, skeletal muscle, skin, adipose, bone, and cartilage. The vascular progenitor cells and/or tissue progenitors cells can be from the same subject into which the engineered tissue composition is grafted. Alternatively, the progenitor cells may be from the same species, or even different species.

Implantation of an engineered tissue or organ construct is within the skill of the art. The matrix and cellular assembly can be either fully or partially implanted into a tissue or organ of the subject to become a functioning part thereof. Preferably, the implant initially attaches to and communicates with the host through a cellular monolayer. Over time, the introduced cells can expand and migrate out of the polymeric matrix to the surrounding tissue. After implantation, cells surrounding the engineered vascularized tissue composition can enter through cell migration. The cells surrounding the engineered tissue can be attracted by biologically active materials, including biological response modifiers, such as polysaccharides, proteins, peptides, genes, antigens, and antibodies which can be selectively incorporated into the matrix to provide the needed selectivity, for example, to tether the cell receptors to the matrix or stimulate cell migration into the matrix, or both. Generally, the matrix is porous, having interconnecting microchannels and/or macrochannels that allow for cell migration, augmented by both biological and physical-chemical gradients. For example, cells surrounding the implanted matrix can be attracted by biologically active materials including one or more of VEGF, fibroblast growth factor, transforming growth factor-beta, endothelial cell growth factor, P-selectin, and intercellular adhesion molecule. One of skill in the art will recognize and know how to use other biologically active materials that are appropriate for attracting cells to the matrix.

Biomolecules can be incorporated into the matrix, causing the biomolecules to be imbedded within. Alternatively, chemical modification methods may be used to covalently link a biomolecule on the surface of the matrix. The surface functional groups of the matrix components can be coupled with reactive functional groups of the biomolecules to form covalent bonds using coupling agents well known in the art such as aldehyde compounds, carbodiimides, and the like. Additionally, a spacer molecule may be used to gap the surface reactive groups in collagen and the reactive groups of the biomolecules to allow more flexibility of such molecules on the surface of the matrix. Other similar methods of attaching biomolecules to the interior or exterior of a matrix will be known to one of skill in the art.

The methods, compositions, and devices of the invention can include concurrent or sequential treatment with one or more of enzymes, ions, growth factors, and biologic agents, such as thrombin and calcium, or combinations thereof. The methods, compositions, and devices of the invention can include concurrent or sequential treatment with non-biologic and/or biologic drugs.

Screening

Another aspect of the invention provides for a method of screening for a molecule that modulates blood vessel formation. This method includes the steps of introducing a tissue progenitor cell and a vascular progenitor cell to a matrix material; culturing the matrix material to form an engineered tissue; contacting the matrix material or the engineered tissue with a candidate molecule; measuring vascularization of the engineered tissue; and determining whether the candidate molecule modulates blood vessel formation in the matrix/tissue relative to a control not contacted with the candidate molecule. Optionally, the screening method can also include implanting the matrix material or the engineered tissue in a subject and inducing endogenous tissue progenitor cells and/or vascular progenitor cells to migrate into the implanted construct.

Preferably, the candidate molecule is part of a test mixture such as a cell lysate, a lysate from a tissue, or a library. A molecule that modulates blood vessel formation can either increase or decrease blood vessel formation (e.g., angiogenesis, vasculogenesis, formation of an immature blood vessel network, blood vessel remodeling, blood vessel stabilization, blood vessel maturation, blood vessel differentiation, or establishment of a functional blood vessel network) in the culture, matrix, tissue, or organ by at least 5%, 10%, 20%, 25%, 30%, 40%, or 50%, 60%, 70%, 80%, 90%, or even by as much as 100%, 150%, or 200% compared to a corresponding control not contacted with the molecule.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### REFERENCES CITED

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention. It shall be understood that any method described in an example may or may not have been actually performed, or any composition described in an example may or may not have been actually been formed, regardless of verb tense used.

### Example 1

### Endothelial Cells Spatially Co-Seeded with MSC Osteoblasts Generate Vascular-Like Structures in Engineered Bone Constructs In Vivo

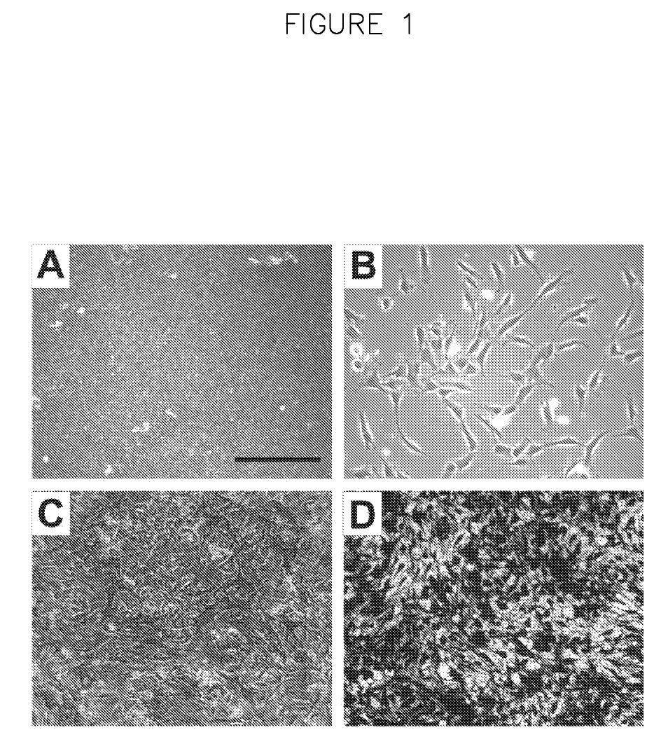

Human bone marrow samples (AllCells, Berkeley, Calif.) were prepared to isolate mesenchymal stem cells (MSCs) and hematopoietic stem cells (HSCs) per previously established methods (Shi et al., 1998; Alhadlaq et al., 2004; Yourek et al., 2004; Marion et al., 2005; Moioli et al., 2006; Troken and Mao, 2006). The initially plated bone marrow content is depicted in FIG. 1A, showing densely populated cells that are known to be heterogeneous (see Alhadlaq and Mao, 2004; Marion and Mao, 2006).

Mesenchymal stem cells can differentiate into osteoblasts. Two distinct cell lineages, human mesenchymal stem cells (MSCs), and human umbilical vein endothelial cells (HUVEC), were used in the engineering of vascularized bone in vivo.

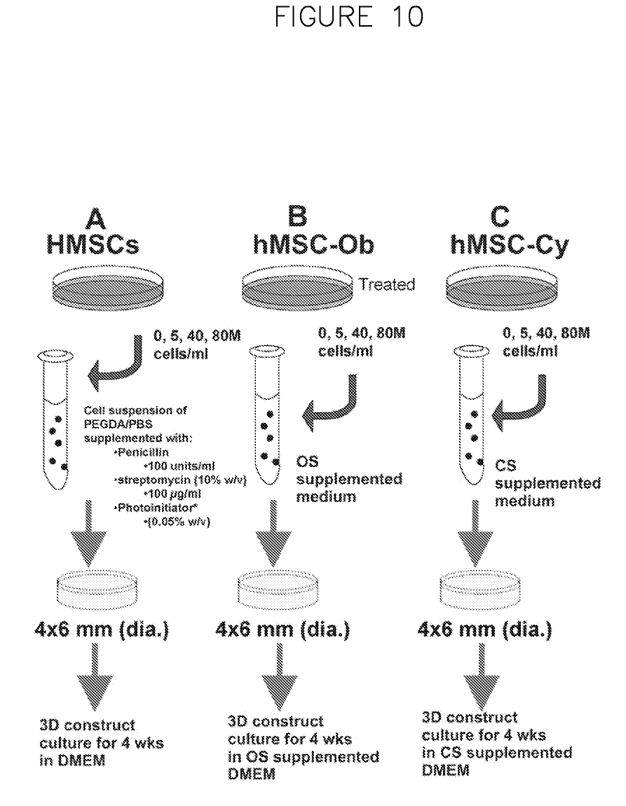

MSCs were isolated from human bone marrow samples, as described above (see e.g., FIG. 1B) (Alhadlaq and Mao, 2003; Alhadlaq et al., 2004; Yourek et al., 2004; Alhadlaq and Mao, 2005; Moioli et al., 2006; Marion and Mao, 2006; Troken and Mao, 2006). A subpopulation of the culture-expanded hMSCs were differentiated into osteogenic cells (Marion et al., 2005; Moioli et al., 2006). The hMSC-derived osteoblasts (hMSC-Ob) were positive to alkaline phosphatase (see e.g., FIG. 1C) and von Kossa (see e.g., FIG. 10).

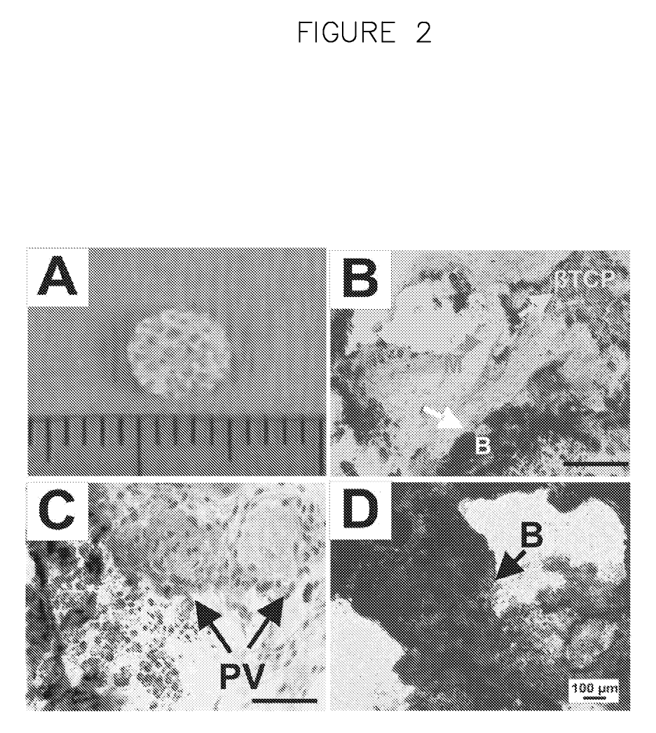

The hMSC-derived osteoblasts (5×10⁶ cells/mL) were seeded in the porous surfaces of β-tricalcium phosphate disks (βTCP; average pore size: 300 μm) in a light vacuum (see e.g., FIG. 2A, light pink regions).

Endothelial cells co-seeded with MSC osteoblasts in engineered bone construct in vivo. Human umbilical vein endothelial cells (HUVEC) were culture-expanded, and then encapsulated in the liquid phase of a Matrigel at a density of 5×10⁶ cells/mL also in a light vacuum at 4° C. (see e.g., FIG. 2A, red dots). Matrigel is a basement membrane polymeric hydrogel that have been widely utilized for endothelial cell adhesion and angiogenesis studies (Abilez et al., 2006; Baker et al., 2006; Bruno et al., 2006; Mondrinos et al., 2006; Rajashekhar et al., 2006).

HUVEC-Matrigel constructs (see e.g., FIG. 2A, red dots) were infused into the pores of PTCP disks that had been seeded with hMSC-derived osteoblasts. The Matrigel was subsequently polymerized by incubation at 37° C. Composite constructs with HUVEC infused, hMSC-Ob-seeded βTCP constructs (see e.g., FIG. 2A) were implanted in the dorsum of severe combined immunodeficient (SCID) mice for 4 wks. Control constructs included hMSC-Ob-seeded βTCP disks, and cell-free pTCP disks.

Upon harvest from in vivo implantation, the retrieved HUVEC infused, hMSC-Ob-seeded PTCP constructs showed areas of mineralization along with the scaffolding material of PTCP in von Kossa stained sections (see e.g., FIG. 2B). Vascular-like lumens formed by endothelial-like cells (see e.g., PV in FIG. 2C) were found among mineral nodules upon hematoxylin and eosin staining (see e.g., FIG. 2C). Substantial regions of PTCP constructs were mineralized upon examination of higher magnification von Kossa section (see e.g., FIG. 2D). Given HUVECs were seeded homogenously in Matrigel, the formation of lumen-like structure aligned by endothelial-like cells (see e.g., FIG. 2C) apparently had involved the reorganization of HUVECs upon in vivo implantation.

These data demonstrate that human MSC osteoblasts and human endothelial cells co-seeded in different spatial regions of a biocompatible material can mediate vascular-like structures among mineral tissue. Thus, several cell lineages can be optimized in engineering vascularized bone, such as HSCs, MSCs, and/or their lineage derivatives including HSC-derived endothelial cells and MSC-derived osteoblasts.

### Example 2

### Bone Marrow Derived Hematopoietic Stem Cells Differentiate to Endothelial-Like Cells In Vitro

For clinical applications, HSCs that can be isolated from bone marrow along with MSCs via minimally invasive approaches are preferred. HSCs have been found to undergo slow expansion (Shih et al., 2000; Li et al., 2004). FGF-2 has been demonstrated to accelerate HSC expansion rate (Wilson and Trump, 2006; Yeoh et al., 2006). It is the inventors experience that HSCs indeed expand at slower rate than MSCs and HUVECs. Alternatively, HSCs can be differentiated into endothelial cells, followed by the expansion of HSC-derived endothelial cells.

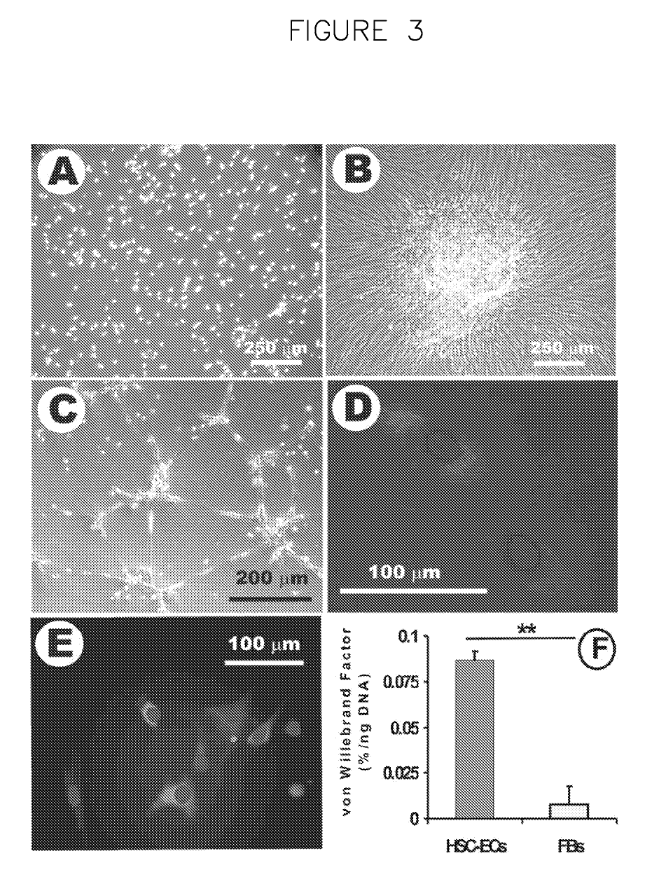

Human bone marrow samples (same as above) were prepared for the isolation of HSCs. CD34 and magnetic bead separation was used to separate non-adherent cells (EasySep, AllCells, Berkeley, Calif.). The isolated CD34 positive cells (CD34+) were deemed to be HSCs. In fibronectin-coated plates, HSCs showed round cell shape (see e.g., FIG. 3A), in sharp contrast to MSCs that assume spindle shape in 2D culture (c.f. e.g., FIG. 1B). Upon transfer of HSCs to new culture plates, endothelial differentiation supplements were added to DMEM, containing VEGF (10 ng/mL), bFGF (1 ng/mL), and IGF-1 (2 ng/mL) (Shi et al. 1998; Shih et al., 2000; Li et al., 2004).

HSCs began to form colonies in approximately 2 weeks (see e.g., FIG. 3B). Further under the stimulation of endothelial differentiation supplements, HSCs differentiated into unconnected cells with the formation of tubular structures that interconnected the cells (see e.g., FIG. 3C). HSC-derived cells were positive to acetylated low density lipoproteins (Ac-LDLs), a typical endothelial cell marker, as evidenced by intracellular localization of Ac-LDL fluorescence (see e.g., FIG. 3D). HSC-derived endothelial cells also expressed von Willebrand factor (vWF), a marker for native endothelial cells, as evidenced by antibody staining (see e.g., FIG. 3E). HSC-derived endothelial cells (HSC-ECs) expressed significantly higher amount of vWF (see e.g., FIG. 3F, left bar) than control fibroblasts (FBs) (see e.g., FIG. 3F, right bar).

Taken together, these data demonstrate that HSCs isolated from human bone marrow can differentiate into endothelial-like cells, as evidenced by native endothelial cell morphology and markers. These HSC-derived endothelial cells form intercellular tubular connections.

Thus, engineered vascular bone can be generated by a blend of HSCs and MSCs, and/or HSC-derived endothelial cells with MSC-derived osteoblasts. This mimics how native bone is formed by vascular invasion during development. Osteogenesis in the mid-diaphyseal region of long bones is accompanied by blood vessels, an elegant demonstration of the synergistic actions of hematopoietic and mesenchymal stem cells in (vascularized) osteogenesis by nature.

### Example 3

### Growth Factors Induce Angiogenesis in Polymeric Hydrogel In Vivo

It has been demonstrated herein that HSCs and MSCs can differentiate into end cell lineages such as endothelial cells and osteoblasts that constitute some of the building blocks of blood vessels and bone. It has also been demonstrated that vascular-like structures can be engineered in bone scaffolds in vivo. However, existing literature has shown that engineered blood vessels can be leaky due to abnormally high endothelial cell permeability (Richardson et al., 2001; Valeski and Baldwin, 2003). To determine the effects of bFGF on host-derived angiogenesis, angiogenic factor bFGF was delivered to a dense polymeric hydrogel, poly(ethylene glycol) diacrylate (PEGDA), that is known to be impermeable to host derived blood vessels in vivo in previous studies (Alhadlaq and Mao, 2003; Alhadlaq et al., 2004; Alhadlaq and Mao, 2005; Stosich and Mao, 2006).

Suboptimal vascularization can be especially problematic when tissue-engineered bone is scaled up towards clinical applications to heal large, critical size bony defects. Data shown below demonstrates that physical macrochannels and a bioactive factor encapsulated in a polymeric hydrogel induce host-derived angiogenesis.

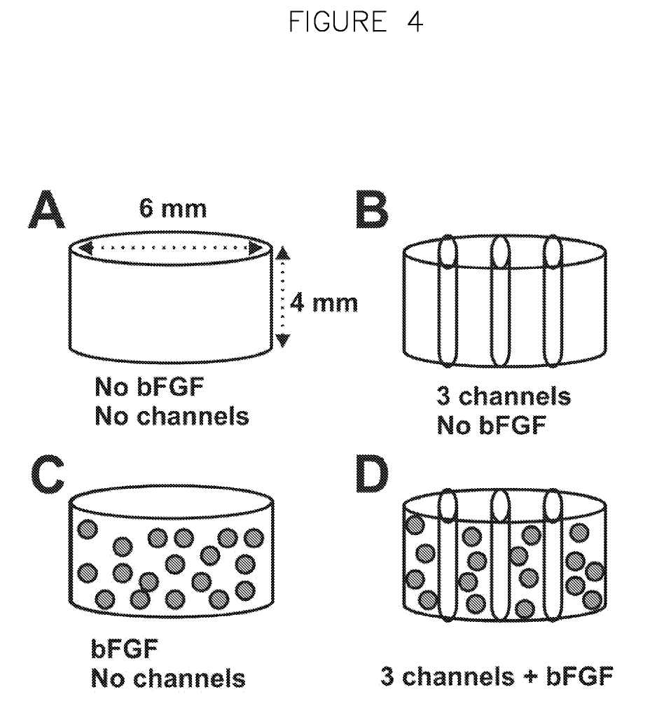

Four configurations were designed in PEG hydrogel (see e.g., FIG. 4) (Stosich et al., 2006). Group 1 consisted of PEG hydrogel alone. A PEG cylinder was created in the dimension of 6×4 mm (dia.×thickness) (see e.g., FIG. 4A) Group 2 consisted of macrochannels alone. A total of 3 macrochannels (1 mm dia. each) were created in photopolymerized PEG cylinder (see e.g., FIG. 4B). Group 3 consisted of bFGF alone. A total of 10 μg/mL bFGF was loaded in the liquid phase of PEG hydrogel, followed by photopolymerization. No macrochannels were created in this group (see e.g., FIG. 4C). Group 4 consisted of bFGF and macrochannels. A total of 10 μg/mL bFGF was loaded in the liquid phase of PEG hydrogel, followed by photopolymerization and the creation of 3 macrochannels (1 mm dia. each) (see e.g., FIG. 4D). No exogenous cells were delivered in any of the four groups. All PEG cylinders had the same dimensions of 6×4 mm (dia.×thickness), and were implanted subcutaneously in vivo in the dorsum of SCID mice (N=8 per group) for 4 wks.

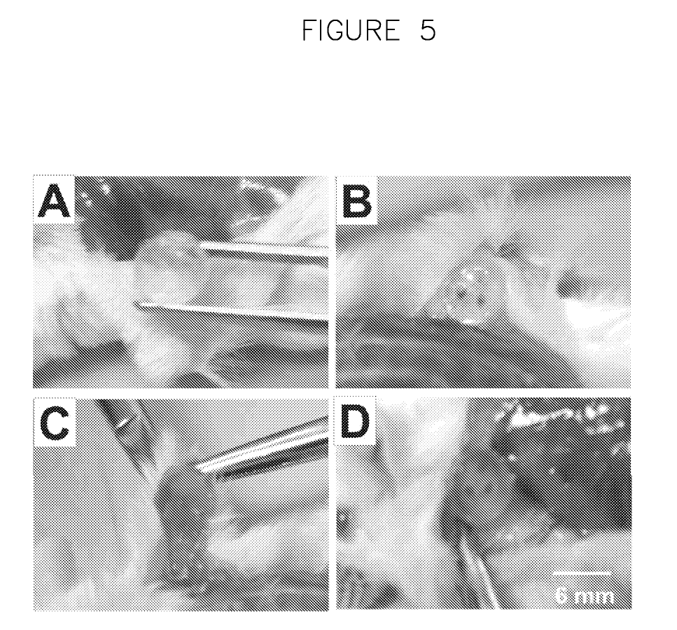

Upon 4-wk in vivo implantation in the dorsum of immunodeficient mice, the following was noted from the analysis of retrieved samples. PEG hydrogel without decorated bFGF or macrochannels showed no macroscopic evidence of vascular infiltration (see e.g., FIG. 5A). In contrast, PEG hydrogel with 3 physical macrochannels showed 3 red dots at the time of in vivo harvest (see e.g., FIG. 5B). Histological and immunohistochemical evidence below suggests that these contain host-derived vascular tissues. PEG hydrogel loaded with bFGF but without macrochannels was darker in overall color (see e.g., FIG. 5C). Histological and immunohistochemical evidence below suggests random areas of host-derived vascular tissue infiltration. And PEG hydrogel with both macrochannels and loaded bFGF not only was darker in overall color, but also showed 3 red dots at the time of in vivo harvest (see e.g., FIG. 5D). Histological and immunohistochemical evidence below demonstrates host-derived vascular tissue infiltration only into the lumens of macrochannels, but not in the rest of the PEG hydrogel.

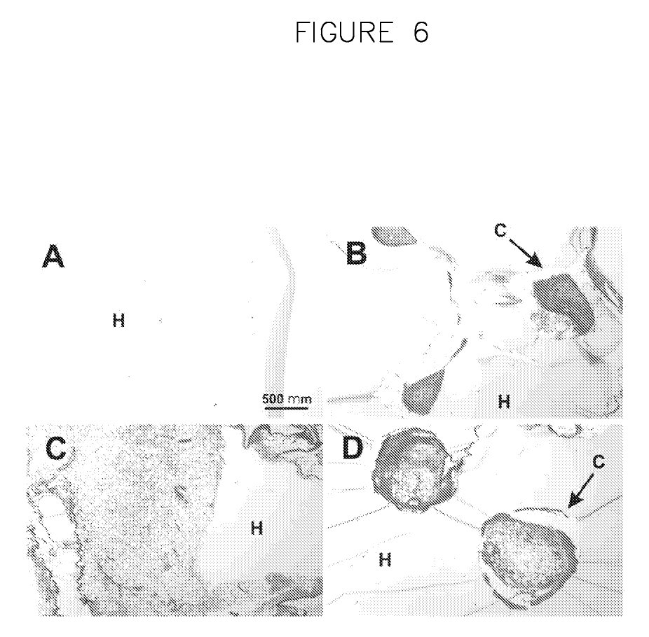

Histological and immunohistochemical findings (Stosich et al. (2006)) are as follows. PEG hydrogel without bFGF or macrochannels (Group 1) showed no host cell invasion or any sign of angiogenesis (see e.g., FIG. 6A), consistent with previous data (Alhadlaq and Mao, 2003; Alhadlaq et al., 2004; Alhadlaq and Mao, 2005; Stosich and Mao, 2005). PEG hydrogel with macrochannels but without bFGF (Group 2 above) demonstrated host cell invasion only into macrochannels, but not in the rest of PEG (see e.g., FIG. 6B). In contrast, bFGF-loaded PEG hydrogel without macrochannels (Group 3) showed apparently random areas of host cell infiltration (see e.g., FIG. 6C). And bFGF-loaded and macrochanneled PEG hydrogel (Group 4 above) demonstrated host cell invasion in macrochannels only, but not the rest of PEG (see e.g., FIG. 6D).

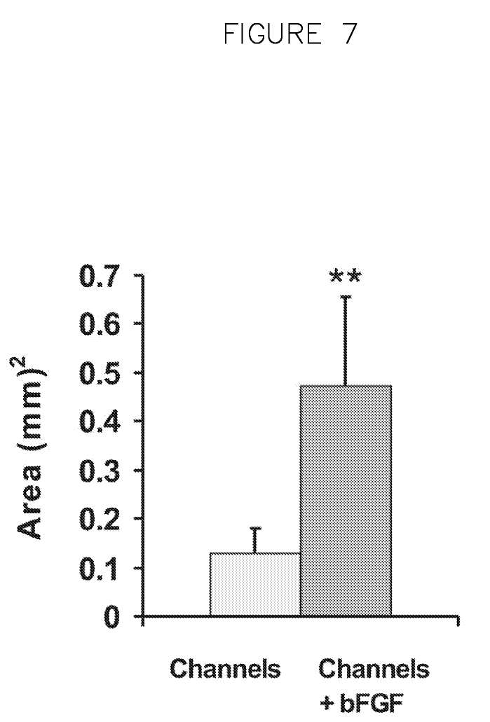

These results show the following. PEG hydrogel with both macrochannels and bFGF had significantly higher amount of host tissue ingrowth at 0.47±0.18 mm2 than bFGF-free PEG hydrogel with macrochannels at 0.13±0.05 mm2 (mean±S.D.; P<0.01; N=8 per group) (see e.g., FIG. 7). Thus, combined physical and bioactive designs in PEG hydrogel promote host tissue ingrowth.

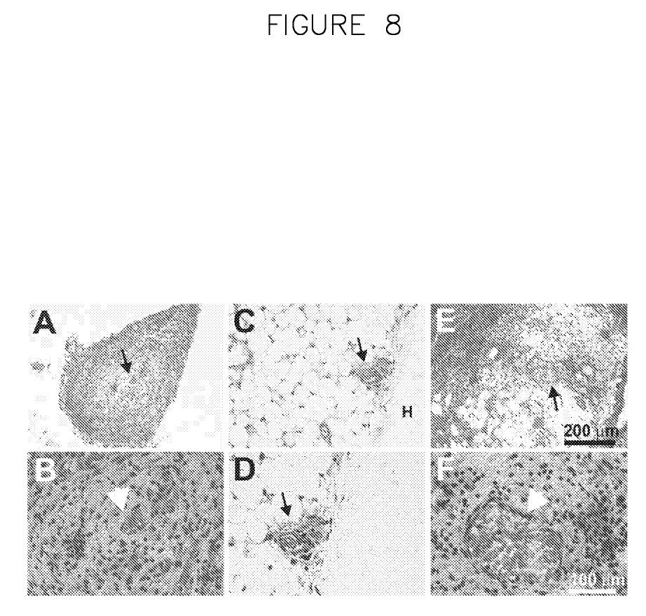

Analysis of higher power images reveal vascular infiltration into PEG hydrogel that otherwise resists host tissue ingrowth (see e.g., FIG. 8).

Host tissue ingrowth occurred in macrochannels with or without bFGF (see e.g., FIG. 8A, 9B and FIG. 8E, 9F). However, as shown in for example FIG. 7, the amount of infiltrating host tissue in macrochannels in bFGF-loaded PEG hydrogel (see e.g., FIG. 8E, 9F) is significantly more than that in macrochanneled PEG hydrogel without bFGF (see e.g., FIG. 8A, 9B). PEG hydrogel loaded with bFGF, but without macrochannels showed sparse connective tissue ingrowth (see e.g., FIG. 8C, 9D). Blood vessel-like structures contained cells resembling red blood cells in blood vessel-like structures that are lined by endothelial-like cells and surrounded by fibroblast-like cells (see e.g., FIG. 8E, 9F).

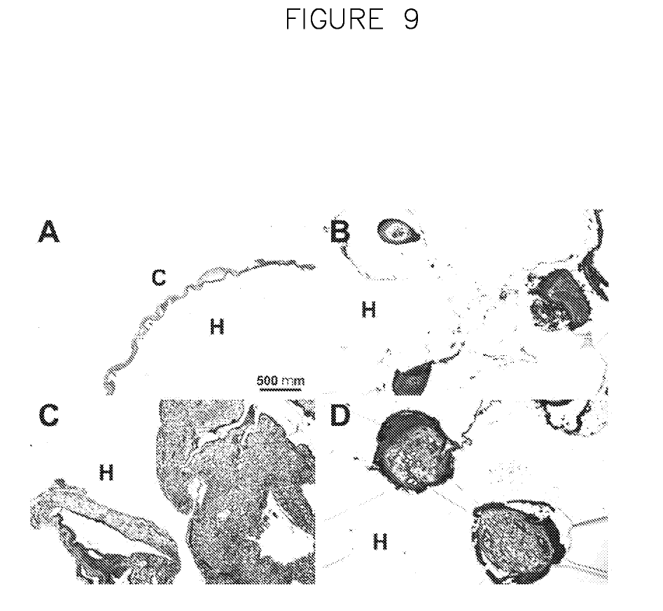

Immunolocalization using anti-vascular endothelial growth factor (VEGF) antibody staining indicates that the ingrowing host tissue is vascular tissue. Strong anti-VEGF staining is present in the infiltrated host tissue in macrochannels with or without bFGF (see e.g., FIG. 9B, 10D). VEGF antibody also labels the host fibrous capsule (see e.g., FIG. 9A) and host tissue infiltrated in PEG hydrogel with bFGF but without macrochannels (see e.g., FIG. 9C).

These data confirm that the blood vessel-like structures (as seen in, e.g., FIG. 8) are host-derived angiogenesis induced by bFGF and/or macrochannels in PEG hydrogel. Angiogenesis is absent in PEG hydrogel without bFGF or macrochannels (see e.g., FIG. 9A). The porosity of PEG hydrogel is likely sufficiently large to allow the diffusion of growth factors and nutrients, as evidenced by the survival of adipogenic, chondrogenic and osteogenic cells in previous work (Burdick et al., 2003; Kim et al., 2003; Alhadlaq et al., 2004; Alhadlaq and Mao, 2005; Moioli et al., 2006; Stosich and Mao, 2006). However, the pore size of PEG hydrogel is not sufficiently large to allow host cell ingrowth unless channels and growth factors such as bFGF are introduced.

Thus, host tissue ingrowth in macrochannels may be useful in directing angiogenesis and host cell invasion along pre-defined routes. Furthermore, augmentation with bFGF, or other angiogenic factors serves to further accelerate ingrowth. These findings support regulation of host-derived angiogenesis and enhancement of the maturation of engineered blood vessels in bone constructs.

### Example 4

### Cell Seeding Density in Tissue Engineering

A pragmatic issue in engineering biological structures is how many cells to incorporate in the scaffold (Moioli and Mao, 2006). When mesenchymal stem cells give rise to osteogenic progenitor cells and end-stage osteoblasts in development, density-dependent inhibition of cell division (previously termed contact inhibition) is a factor for cell survival (Alberts et al., 2002). Too many cells seeded in an engineered tissue scaffold may create shortage of locally available mitogens, growth factors and survival factors, potentially leading to apoptosis and causing unnecessary waste of in vitro cell expansion time (Moioli and Mao, 2006). On the other hand, too few cells seeded in an engineered tissue scaffold may lead to poor regeneration outcome. Thus, the optimal density of HSCs, MSCs and their lineage derivatives should be determined in order to maximize the regenerative outcome of engineered vascular bone (see e.g., FIG. 10).

Herein is reported the effects of varying the initial cell seeding density of MSCs, MSC-derived osteoblasts, and MSC-derived chondrocytes. Human MSCs were isolated from each of several bone marrow samples of multiple, healthy donors, expanded in monolayer culture and differentiated separately into chondrogenic cells and osteogenic cells as above and per prior methods (Alhadlaq et al., 2004; Marion et al., 2005; Yourek et al., 2005; Moioli et al., 2006) (see e.g., FIG. 10). Four cell densities were adopted for each cell lineage, hMSCs, hMSC-Ob and hMSC-Cy: 0×10⁸ cells/mL, 5×10⁶ cells/mL, 40×10⁸ cells/mL, and 80×10⁶ cells/mL. Intermediate cell seeding density of 20×10⁶ cells/mL was previously investigated (Alhadlaq and Mao, 2005). 0×10⁶ cells/mL=cell-free construct. Cell suspension for each cell density and lineage was encapsulated in the aqueous phase of PEG hydrogel, followed by photopolymerization, and continuous culture of 3D PEG constructs for 4 wks (see e.g., FIG. 10).

Upon continuous incubation of 3D PEG hydrogel constructs separately in DMEM, osteogenic supplemented DMEM or chondrogenic supplemented DMEM for 4 weeks with frequent medium changes, histological staining and biochemical assays were performed. Osteogenic medium contained 100 nM dexamethasone, 50 μg/ml ascorbic acid and 100 mM β-glycerophosphate, whereas chondrogenic supplemented medium contained 10 ng/ml TGFβ3 (details below).

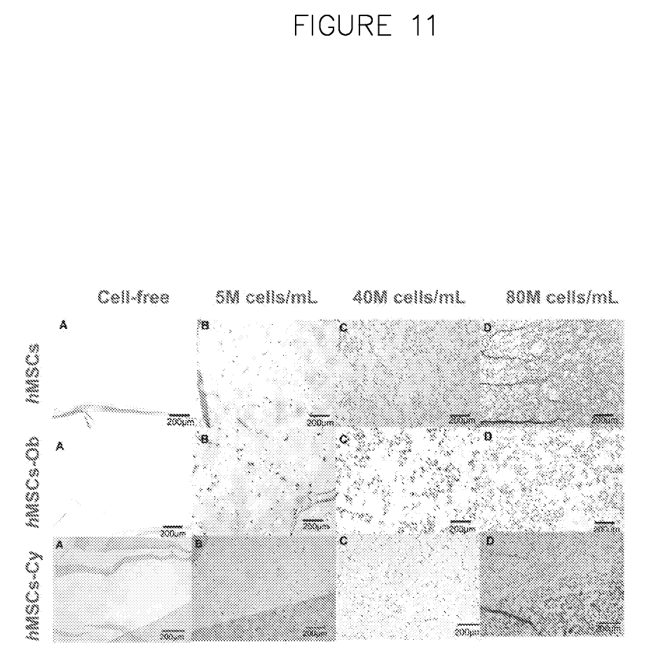

Results showed that the initial cell seeding densities were maintained in PEG hydrogel upon 4-wk incubation in corresponding media of DMEM, osteogenic supplemented DMEM and chondrogenic supplemented DMEM (see e.g., FIG. 11) (see e.g., Troken and Mao, 2006). FIG. 11 depicts exemplary results of H&E staining and demonstrates histological observation of various densities of hMSCs (top row), hMSC-derived osteoblasts (middle row), and hMSC-derived chondrocytes (bottom row) encapsulated in PEG hydrogel and subjected to 4-wk 3D construct culture. In general, the end-point cell densities in PEG hydrogel scaffolds followed similar initial cell seeding density patterns at 5M cells/ml, 40M cells/ml and 80M cells/ml (5 M/ml=5 million cells per mL of cell suspension).

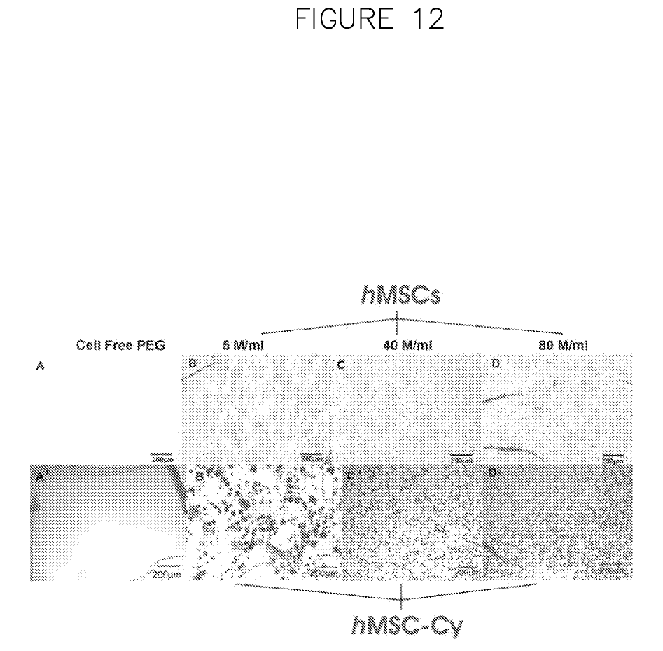

The hMSC-derived chondrocytes (hMSC-Cy) maintained not only their chondrogenic phenotype upon 4-wk incubation in PEG hydrogel, but also their corresponding initial cell seeding densities (see e.g., FIG. 12, safranin O staining) (see e.g., Troken and Mao, 2006). Safranin O is a cationic dye that binds to cartilage-related glycosaminoglycans such as keratin sulfate and chondroitin sulfate, and has been widely used to label native articular and growth plate cartilage (see e.g., Mao et al., 1998; Wang and Mao, 2002; Sundaramurthy and Mao, 2006). In contrast, although hMSCs maintained their initial cell seeding densities in PEG hydrogel upon 4-wk incubation, they were negative to safranin O staining (see e.g., FIG. 12).

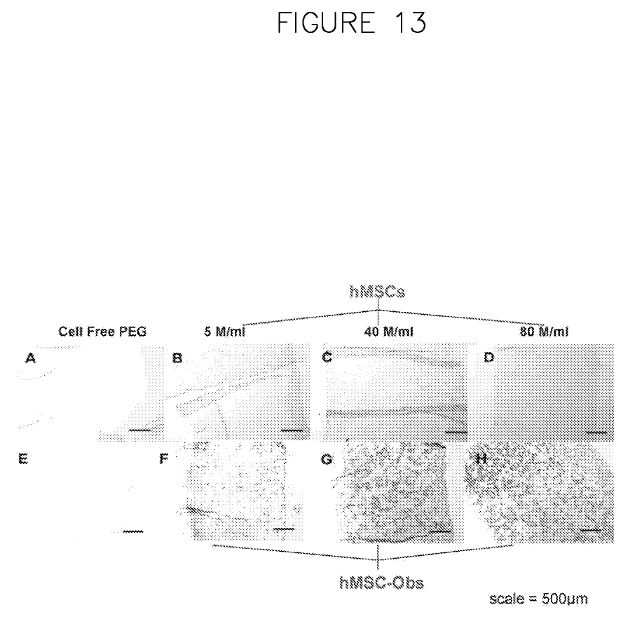

The hMSC-derived osteoblasts (hMSC-Ob) maintained not only their osteogenic phenotype upon 4-wk incubation in PEG hydrogel, but also their corresponding initial cell seeding densities (see e.g., FIG. 13, von Kassa staining) (Troken and Mao, 2006). Von Kossa is conventionally used to label mineral formation in both native osteogenesis and tissue-engineered osteogenesis (see e.g., Alhadlaq and Mao, 2003; Alhadlaq et al., 2004; Marion et al., 2005; Moioli et al., 2006). In contrast, although hMSCs maintained their initial cell seeding densities in PEG hydrogel upon 4-wk incubation, they were negative to von Kossa staining (see e.g., FIG. 13).

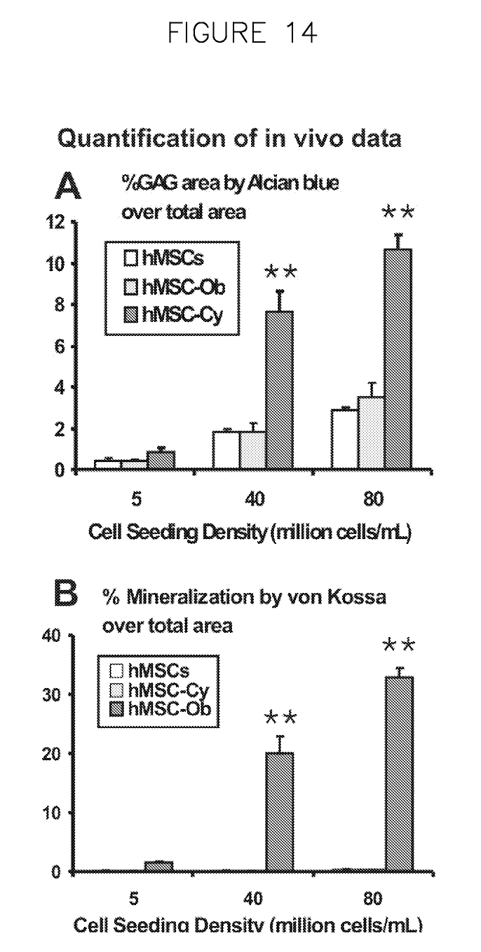

Upon implantation of PEG hydrogels encapsulating the same densities of hMSCs, hMSC-Ob and hMSC-Cy in nude rats, the in vivo data showed that increasing initial cell seeding density led to increasing amount of matrix formation by hMSC-derived osteoblasts and hMSC-derived chondrocytes (see e.g., FIG. 14) extending the in vitro data presented above (see e.g., FIGS. 11-14).

This cell density experiment confirms previous in vivo findings by comparing two cell densities at 5 million cells/mL and 20 million cells/mL (Alhadlaq et al., 2004; Alhadlaq and Mao, 2005) in that the regenerative outcome of a higher cell seeding density, e.g. at 20 million cells/mL is superior to seeding density at 5 million cells/mL. However, excessively high cell seeding density may elicit issues such as the shortage of nutrients, abnormal cell-cell contact, apoptosis, and unnecessary waste of in vitro cell expansion time (Moioli and Mao, 2006). Generally, shortest ex vivo expansion time is preferred.

These cell density experiments demonstrate that optimization of seeding densities of cells encapsulated in tissue-engineering scaffolds can maximize the regenerative outcome (see Alhadlaq et al., 2004; Alhadlaq and Mao, 2005; Troken and Mao, 2006).

### Example 5

### Optimal Ratios Between HSCs and MSCs

The following experiments investigate the ratios between HSCs and MSCs that are optimal to the engineering of vascularized bone.

TABLE 2
The relative contribution of HSCs and MSCs to the engineering of
vascularized bone are investigated with a factorial design approach in
an 8 × 8 × 2 design: cell ratios (8) × sample size (8) × in vivo
implantation times (2). The total number of cells (HSCs and MSCs
combined) in each scaffold in vitro is kept constant at 8 × 10⁶ cells/
mL, while the relative ratios of HSCs and MSCs vary from 1:1 to 1:15,
thus enabling the determination of the relative contribution of HSCs
and MSCs towards engineered vascular bone.

| | | | | Sample | In vivo |
|---|---|---|---|---|---|
| | HSCs | MSCs | | Size (# of | implantation |
| | (# of | (# of | HSC:MSC | nude rats | duration |
| Grp | cells/mL) | cells/mL) | ratio | per group) | (wks) |
| 1 | 0 | 8 × 10⁶ | 0 | 8 | 8, 16 |
| 2 | 0.5 × 10⁶ | 7.5 × 10⁶ | 1:15 | ″ | ″ |
| 3 | 2 × 10⁶ | 6 × 10⁶ | 1:3 | ″ | ″ |
| 4 | 4 × 10⁶ | 4 × 10⁶ | 1:1 | ″ | ″ |
| 5 | 6 × 10⁶ | 2 × 10⁶ | 3:1 | ″ | ″ |
| 6 | 7.5 × 10⁶ | 0.5 × 10⁶ | 15:1 | ″ | ″ |
| 7 | 8 × 10⁶ | 0 | 0 | ″ | ″ |
| 8 | | Cell-free | | ″ | ″ |
| | | scaffold | | | |

| | |
|---|---|
| Total number | 128 = 8 groups × 8 |
| of rats | samples per group × |
| | 2 time points |

Human HSCs and MSCs are isolated from each of several bone marrow samples per studies described above, and previously established methods (Alhadlaq and Mao, 2003; Alhadlaq et al., 2004; Yourek et al., 2004; Alhadlaq and Mao, 2005; Moioli and Mao, 2006; Moioli et al., 2006; Marion and Mao, 2006; Troken and Mao, 2006; Stosich et al., 2006). HSCs and MSCs from a single donor are used in each construct to eliminate any potential immunorejection issues. HSCs are seeded homogeneously in Matrigel as in studies described above, and infused into the pores of βTCP that has been pre-seeded with MSCs. Cell-scaffold constructs are implanted in the dorsum of nude rats, which do not reject human cells. The rationale for 8 and 16 weeks of in vivo implantation is that angiogenesis, if it takes place, is anticipated to occur within this time frame per previous experience (Stosich et al., 2006).

The implanted samples are harvested, and subjected to the analyses outlined in Table 3 below.

TABLE 3
Outcome assessments and success criteria of engineered vascular bone. Detailed
methods for these techniques are discussed below.

| | | | | | Immunohistochemistry and | | |
|---|---|---|---|---|---|---|---|
| | Histology | | Structural analysis | | biochemical analysis | | Mechanical properties of |
| | Bone | Vessel | Bone | Vessel | Bone | Vessel | vascularized bone |
| Parameters | H&E | H&E | μCT | Blood | Osteopontin, | α-SMA, vWF, | Microindentation with |
| | Von | Masson's | Digital X ray | vessel # | osteocalcin, | Connexin-43, | atomic force microscopy |
| | Kossa | trichrome | Histomorpho- | and | bone | PECAM, | Conventional mechanical |
| | | | metry | average | sialoprotein | VEGFR, | testing with biaxial capacity |
| | | | | diameter | | KDR/VEGFR- | |
| | | | | | | 2/Flk-1 | |

| | | | | |
|---|---|---|---|---|
| Success | Presence of blood | Mineralized tissue | Presence of these osseous | Overall mechanical |
| criteria | vessel-like | formation resembling | and angiogenic markers | properties at least 50% of |
| | structures | trabecular bone | Quantitative biochemical | native trabecular bone |
| | Presence of | structures | analysis of bone and | |
| | mineralized tissue | | angiogenic markers | |
| References | Alhadlaq and Mao, | Kopher and Mao, 2003 | Mao et al., 1998 | Radhakrishnan and Mao, |
| | 2003 | Mao et al., 2003 | Alhadlaq and Mao, 2005 | 2004 |
| | Alhadlaq et al., | Vij and Mao, 2006 | Sundaramurthy and Mao, 2006 | Allen and Mao, 2004 |
| | 2004 | Ho et al., 2006 | Landesberg et al., 1999 | Guo, 2000 |
| | Sundaramurthy | Takai et al., 2006 | Stosich et al., 2006 | Guo and Kim, 2002 |
| | and Mao, 2006 | Meinel et al., 2005 & | Xin et al., 2006 | Vunjak-Novakovic et al., |
| | Vij and Mao, 2006 | 2006 | | 1999 |

H&E: Hematoxylin and Eosin, general histology stain for differentiating multiple tissues;
Masson's Trichrome: histology stain for blood vessels,
OCN: Osteocalcin, adhesion protein for osteoblasts, late marker for osteogenic differentiation,
OPN: Osteopontin, adhesion protein for osteoblasts, late marker for osteogenic differentiation,
vWF: von Willebrand factor, surface glycoprotein found on endothelial cells, late marker for endothelial cell differentiation,
VEGFR: Vascular endothelial growth factor receptor, early-late marker for endothelial cell differentiation,
KDR/VEGFR-2/Flk-1: Vascular endothelial growth factor receptor 2, early-late marker for endothelial cell differentiation.

Engineered vascular bone volume is quantified by digital X-ray and pCT with detailed methods described below. Mechanical analyses of engineered vascular bone are performed using microindentation with atomic force microscopy (AFM) as well as compressive and shear tests using conventional mechanical testing. Micromechanical properties of engineered vascular bone are of interest and can be readily studied by AFM, but cannot be obtained by macroscale mechanical testing with Instron or MTS. However, MTS is capable of testing the overall compressive and shear properties of engineered vascular bone, which can not be tested by AFM. Thus, AFM and MTS are complementary mechanical testing approaches for engineered vascular bone. All numerical data are subjected to statistical analyses. For normal data distribution, Analysis of Variance (ANOVA) with Bonferroni tests are used. If data distribution is skewed, nonparametric tests such as Kruskal-Wallis analysis of variance are used. Statistical significance is at an alpha level of 0.05.

Autologous cells and allogenic cells have both been used in tissue engineering. Presented herein is a model of autologous cells in tissue engineering (human cells implanted in nude rats). The nude rat serves as a simulating human “incubator”. In comparison with allogenic cells, autologous cells have several critical advantages such as lack of immunorejection and pathogen transmission. Allogenic cells can be readily made available for the recipient, thus eliminating the time required for cell manipulation in association with autologous cells. However, immunosuppressant drugs may need to be administered and may complicate the outcome of tissue engineering of vascularized bone. Selection of bone marrow stem cells is based at least in part on the observation that bone marrow-derived MSCs and HSCs have been well characterized, and have the potential to engineer vascularized bone, as demonstrated in studies described above. Adipose derived stem cells have been recently reported and may provide an alternative to bone marrow derived cells.

### Example 6

### Optimal Cell Densities Between HSCs, MSCs and Their Lineage Derivatives Maximize the Outcome of Engineering Vascularized Bone

Although HSCs and MSCs function synergistically in vascularized bone development, several other cell lineages are also involved in vascular osteogenesis including endothelial cells and osteoblasts. Osteoblasts are one of the MSC-derived end stage cells. Accordingly, there is a need to investigate whether the engineering of vascularized osteogenesis is maximized by blending HSCs with MSC-derived osteoblasts, as well as MSCs with HSC-derived endothelial cells. Whether endothelial cells are derived from MSCs, HSCs or other progenitor cells is not well understood (Yin and Li, 2006). Endothelial-like cells are differentiated from HSCs, thus providing a viable cell source to study the involvement of HSC-derived endothelial cells in engineered vascular bone.

The following experimental design investigates cell seeding densities of not only HSCs and MSCs, but also their lineage derivatives including HSC-derived endothelial cells and MSC-derived osteoblasts in the engineering of vascularized bone.

TABLE 4
Experimental design, Experiment 1 - HSCs and MSC-derived osteoblasts. The
relative contribution of HSCs and MSC-derived osteoblasts in the engineering of
vascularized bone are investigated with a factorial design approach in an 8 × 8 × 2
design: cell density (8) × sample size (8) × in vivo implantation times (2).

| | HSCs | MSC-derived | HSC:MSC- | Sample Size | In vivo implantation |
|---|---|---|---|---|---|
| | (# of | osteoblasts | Ob | (# of nude rats per | duration |
| Group | cells/mL) | (# of cells/mL) | ratio | group) | (wks) |
| 1 | 0 | 8 × 10⁶ | 0 | 8 | 8, 16 |
| 2 | 0.5 × 10⁶ | 7.5 × 10⁶ | 1:15 | ″ | ″ |
| 3 | 2 × 10⁶ | 4 × 10⁶ | 1:3 | ″ | ″ |
| 4 | 4 × 10⁶ | 2 × 10⁶ | 1:1 | ″ | ″ |
| 5 | 6 × 10⁶ | 1 × 10⁶ | 3:1 | ″ | ″ |
| 6 | 7.5 × 10⁶ | 0.5 × 10⁶ | 15:1 | ″ | ″ |
| 7 | 8 × 10⁶ | 0 | 0 | ″ | ″ |
| 8 | | Cell-free | | ″ | ″ |
| | | scaffold | | | |

| | |
|---|---|
| Total number | 128 = 8 groups × 8 samples |
| of rats | per groups × 2 time points |

TABLE 5
Experimental design, Experiment 2 - MSCs and HSC-derived endothelial cells. The
relative contribution of MSCs and HSC-derived endothelial cells to engineer vascularized
bone is investigated with a factorial design approach in an 8 × 8× 2 design:
cell density (8) × sample size (8) × in vivo implantation times (2).

| | MSCs | HSC-derived | HSC:MSC- | Sample Size | In vivo implantation |
|---|---|---|---|---|---|
| | (# of | endothelial cells | Ob | (# of nude rats per | duration |
| Group | cells/mL) | (# of cells/mL) | ratio | group) | (wks) |
| 1 | 0 | 8 × 10⁶ | 0 | 8 | 8.16 |
| 2 | 0.5 × 10⁶ | 7.5 × 10⁶ | 1:15 | ″ | ″ |
| 3 | 2 × 10⁶ | 4 × 10⁶ | 1:3 | ″ | ″ |
| 4 | 4 × 10⁶ | 2 × 10⁶ | 1:1 | ″ | ″ |
| 5 | 6 × 10⁶ | 1 × 10⁶ | 3:1 | ″ | ″ |
| 6 | 7.5 × 10⁶ | 0.5 × 10⁶ | 15:1 | ″ | ″ |
| 7 | 8 × 10⁶ | 0 | 0 | ″ | ″ |
| 8 | | Cell-free scaffold | | ″ | ″ |

| | |
|---|---|
| Total number | 128 = 8 groups × 8 samples |
| of rats | per groups × 2 time points |

Human HSCs and MSCs are isolated from each of several bone marrow samples per methods in studies described above, and previously established methods (Alhadlaq and Mao, 2003; Alhadlaq at, 2004; Yourek et al., 2004; Alhadlaq and Mao, 2005; Moioli and Mao, 2006; Marion and Mao, 2006; Troken and Mao, 2006; Stosich et al., 2006). HSCs and MSCs from a single donor are used in each cell-seeded construct to eliminate potential immunorejection issues. For Experiment 1, MSCs are differentiated into osteoblast-like cells per previously established approaches (Alhadlaq and Mao, 2003; Alhadlaq et al., 2004; Yourek et al., 2004; Alhadlaq and Mao, 2005; Moioli and Mao, 2006; Troken and Mao, 2006; Marion and Mao, 2006). For Experiment 2, HSCs are differentiated into endothelial-like cells per approaches in studies described above. HSC-derived endothelial cells are seeded homogeneously in Matrigel as in studies described above, and infused into the pores of βTCP that has been pre-seeded with MSC-derived osteoblasts. For Experiment 2, MSCs are first seeded in the pores of βTCP prior to the seeding of HSC-derived endothelial cells in Matrigel. For both Experiments 1 and 2, cell-scaffold constructs are implanted in nude rats, which do not reject human cells. The rationale for 8 and 16 weeks of in vivo implantation is that angiogenesis, if it takes place, is anticipated to occur within this time frame per our previous experience (Stosich et al., 2006).

Outcome assessment and Data analysis and statistics are as described above.

Co-seeding of HSC-derived endothelial cells with MSC-derived osteoblasts or chondrocytes can also occur.

### Example 7

### Angiogenic Growth Factors Promote the Maturation of Blood Vessels in HSC- and MSC-Derived Vascular Bone

An engineered vascular system must function properly such as providing proper nutrient supply, oxygenation, gas exchange and cell supply within the newly formed bone tissue. Angiogenesis involves a cascade of events including endothelial cell activation, migration and proliferation. Engineered blood vessels can be leaky due to abnormally high endothelial permeability (Richardson et al., 2001; Valeski and Baldwin, 2003). It is known that a number of angiogenic growth factors regulate the formation of blood vessels in native development (Thurston, 2002; Ehrbar et al., 2003; Valeski and Baldwin, 2003; Ferrara, 2005). VEGF is highly expressed during the first few days of angiogenesis in bone (Nissen et al., 1996; Hu et al., 2003; Bohnsack and Hirschi, 2004; Ferrara, 2005). PDGF effects on vasculature after the actions of VEGF, and enhances the maturation of vascular endothelial cells (Darland and D'Amore, 1999; Richardson et al., 2001; Bohnsack and Hirschi, 2004). Another potential of “leaky” blood vessels in tissue engineering is due to a paucity of associated mural cells such as pericytes and smooth muscle cells. PDGF has been shown to induce the recruitment of mural cells (Darland and D'Amore, 1999; Yancopoulos et al., 2000; Valeski and Baldwin, 2003; Ferrara, 2005). Accordingly, the delivery of PDGF also targets the maturation of engineered neovasculature by recruiting mural cells.

To identify the optimal doses of VEGF and PDGF in enhancing the maturation of engineered blood vessels from HSCs or HSC-derived cells, doses that are higher and lower than the perceived physiological doses are explored. Rapid release of VEGF is desirable in the recruitment and proliferation of angiogenic cells (Nissen et al., 1996; Hu et al., 2003; Ferrara, 2005). Hence, VEGF is soaked in βTCP disks for rapid release within the first few hours or days of in vivo implantation. PDGF's action follows VEGF and promotes not only the maturation of endothelial cells, but also serves as chemo-attractant for mural cells (Darland and D'Amore, 1999; Yancopoulos et al., 2000; Valeski and Baldwin, 2003; Ferrara, 2005). Hence, PDGF is encapsulated in microspheres for sustained release without an initial burst phase (Moioli et al., 2006) so to allow gradual and sustained release of PDGF following the actions of more rapidly released VEGF. The encapsulation of PDGF microspheres in Matrigel will further retard its release rate per experience in studies described above.

TABLE 6
Experimental design to enhance the maturation of neovasculature in
engineered bone. HSC-EC: hematopoietic stem cell derived endothelial
cells; MSC-Ob: mesenchymal stem cell derived osteoblasts. The outcome
will be investigated with a factorial design approach in an 8 × 5 ×
2 design: sample size (8) × growth factor doses (5) × in vivo
implantation times (2).

| | VEGF | | | | |
|---|---|---|---|---|---|
| | soaked in | PDGF | Cells | Sample | |
| | hydrogel | (ng/mL) in | HSC:MSC | size | In vivo |
| | μg per | micro- | HSC-EC:MSC | (rats per | implantation |
| Groups | construct | spheres | MSC-Ob:HSC | group) | (weeks) |
| 1 | 0 | Plasebo | Optimized | 8 | 8, 16 |
| | | micro- | from | | |
| | | spheres | Alms 1 & 2 | | |
| 2 | 1 | 10 | Optimized | ″ | ″ |
| | | | from | | |
| | | | Alms 1 & 2 | | |
| 3 | 1 | 100 | Optimized | ″ | ″ |
| | | | from | | |
| | | | Alms 1 & 2 | | |
| 4 | 10 | 10 | Optimized | ″ | ″ |
| | | | from | | |
| | | | Alms 1 & 2 | | |
| 5 | 1 | 1 | Optimized | ″ | ″ |
| | | | from | | |
| | | | Alms 1 & 2 | | |

| | |
|---|---|
| Total number | 80 = 8 samples × |
| of rats | 5 groups × 2 |
| | time points |

VEGF is soaked in Matrigel, followed by infusion into the pores of βTCP, for rapid release. PDGF is encapsulated in PLGA microspheres by double emulation technique with technical details described herein and per previous methods (Moioli et al., 2006). PDGF is released at a slow rate and without an initial burst phase. The procedures for cell seeding are the same as in Example 1, prior to the loading of growth factors.

Outcome assessment and data analysis and statistics are as described above.

The doses of VEGF and PDGF are obtained from studies described above and existing literature (see e.g., Darland and D'Amore, 1999; Yancopoulos et al., 2000; Richardson et al., 2001; Valeski and Baldwin, 2003; Ferrara, 2005). Alternatively, bFGF can be used in replacement of VEGF, also given previous experience (Stosich et al., 2006). The addition of multiple growth factors to cell delivery creates a complex system, although this is how native angiogenesis and osteogenesis take place. An alternative to soaking VEGF in Matigel is lyophilization to βTCP. PLGA is known to generate acidic byproducts during degradation. However, since only small amount of PLGA is used in the fabrication of microspheres, the acidic byproduct issue is not substantial, and has been minimal in previous work (Moioli et al., 2006). PDGF is anticipated to recruit vascular smooth muscle cells as demonstrated by existing literature (Darland and D'Amore, 1999; Yancopoulos et al., 2000; Valeski and Baldwin, 2003; Ferrara, 2005). The lowest effective dose is generally adopted in consideration of the economics of ultimate clinical therapies. Upon the incorporation of HSCs and MSCs to engineer vascularized bone, it is probable that the amount of needed angiogenic growth factors is not as high as without the incorporation of HSCs and MSCs (and/or their lineage derivatives). Logically, HSCs and MSCs and/or their lineage derivatives likely also mediate necessary angiogenic growth factors.

### Example 8

### Optimized Delivery of HSCs, MSCs and/or Angiogenic Growth Factors Effectively Heal Critical-Size Calvarial Defects

Experiments described above provide for optimized cell- and/or growth-factor-based approaches towards engineering vascularized bone using an ectopic osteogenesis approach. Calvarial bone defects represent substantial clinical needs and also an orthotopic site for testing the optimized cell- and/or growth-factor-based approaches in engineering vascularized bone.

This experiment provides an orthotopic bone defect environment to test whether the optimized conditions determined via methods outlined above can heal critical size calvarial defects more effectively than any constituent alone and/or conventional bone tissue engineering approaches. Calvarial defects represent a different experimental model from the ectopic implantation site utilized in experiments described above.

TABLE 7
Experimental design to heal critical size calvarial defects
with optimized approaches to engineer vascularized bone.
HSC-EC: hematopoietic stem cell derived endothelial cells;
MSC-Ob: mesenchymal stem cell derived osteoblasts. The
outcome is investigated with a factorial design approach in
an 8 × 7 × 2 design: cell constituents (7) × sample
size (8) × in vivo implantation times (2).

| | | Cell delivery | | |
|---|---|---|---|---|
| | VEGF and | Cell density | Sample | |
| | PDGF | and ratios | size | In vivo |
| | delivery | optimized from | (rats per | implantation |
| Groups | dose | Aims 1 and 2 | group) | (weeks) |
| 1 | Optimized from | HSCs | 8 | 8, 16 |
| | Example 3 | | | |
| 2 | Optimized from | MSCs | ″ | ″ |
| | Example 3 | | | |
| 3 | Optimized from | HSCs and MSCs | ″ | ″ |
| | Example 3 | | | |
| 4 | Optimized from | HSCs and MSC-Ob | ″ | ″ |
| | Example 3 | | | |
| 5 | Optimized from | MSCs and HSC-EC | ″ | ″ |
| | Example 3 | | | |
| 6 | Optimized from | Cell-free βTCP | ″ | ″ |
| | Example 3 | | | |
| 7 | None | Cell-free βTCP | ″ | ″ |

Total number of rats 112 = 8 samples × 7 groups × 2 time points

Outcome assessment is as described above. In addition, calcein and alizarin will be injected i.p. 2 and 1 wk prior to the scheduled sacrifice time points for subsequent identification of newly formed calvarial bone (Parfitt at al, 1987; Kopher and Mao, 2003; Clark et al., 2005). Data analysis and statistics is as described above. In addition, bone formation rate (BFR) and mineral apposition rate (MAR) are quantified by fluorescence microscopy with dynamic histomorphometry (Parfitt et al., 1987; Kopher and Mao, 2003; Clark et al., 2005).

The delivered duel growth factors may have complex effects on not only delivered cell lineages, but also the invading host cells in the calvarial environment. For example, in addition to promoting angiogenesis, PDGF facilitates the proliferation of osteoprogenitor cells (Park et al., 2000). This sophisticated system is necessary for providing an intervening tool without which critical size calvarial defects do not heal. The doses of duel growth factors (VEGF and PDGF here), although optimized in Example 3 above, may need to be modified in light of endogenous growth factors that may be present in the calvarial defect model.

### Example 9

### Isolation and Culture-Expansion of Bone Marrow Derived Hematopoletic Stem Cells and Mesenchymal Stem Cells

Isolation of bone marrow derived hematopoietic stem cells and mesenchymal stem cells follows the approaches as described in the above studies and our previously developed methods (see e.g., Alhadlaq and Mao, 2003; Alhadlaq et al., 2004; Alhadlaq et al., 2005; Stosich and Mao, 2005; Marion et al., 2005; Yourek et al., 2005; Moioli et al., 2006; Marion and Mao, 2006; Stosich et al., 2006). Bone marrow samples donated by anonymous adults are obtained commercially (AllCells, Berkeley, Calif.) as in previous work (Alhadlaq et al., 2005; Marion et al., 2005; Yourek et al., 2005). A portion of each bone marrow sample is used to isolate mesenchymal stem cells (hMSCs) using negative selection techniques of the RosetteSep kit (AllCells, Berkeley, Calif.). The isolated MSCs are culture-expanded using Dulbecco's Modified Eagle's Medium-Low Glucose (DMEM-LG; Sigma, St. Louis, Mo.) supplemented with 10% fetal bovine serum (FBS) (Biocell, Rancho Dominguez, Calif.) and 1% antibiotic (1× Antibiotic-Antimycotic, including 100 units/ml Penicillin G sodium, 100 μg/ml Streptomycin sulfate and 0.25 μg/ml amphotericine B (Gibco, Invitrogen, Carlsbad, Calif.) (Alhadlaq et al., 2005; Marion et al., 2005; Yourek et al., 2005, Moioli et al. 2005; Stosich et al., 2006). The hMSCs are expanded no more than 3 passages per bone marrow sample for each experiment. In previous experience, it is rarely necessary to expand more than 3-5 passages. Cultures are incubated in 95% air/5% CO₂ at 37° C.

The same bone marrow sample per donor is utilized to isolate hematopoietic stem cells. Positive selection is carried out using CD34 antibodies attached to magnetic beads (RosetteSep). Flow cytometry of the purified cells is used to determine the percent of the isolated cells that are CD34 positive (CD34+). Viability of the cells is also evaluated by Trypan Blue exclusion. CD34+ cells are isolated from initially non-adherent cells by incubation in 96-well fibronectin coated plastic dishes at 37° C. for 3 days with 10% FBS added to IMDM (HSC growth medium), followed by the collection of the non-adherent cells (Shi et al. 1998). The non-adherent cells are removed and transferred to fresh wells. This process is repeated twice upon which time the suspended cells remaining are plated and allowed to adhere to fibronectin-coated plates.

### Example 10

### Differentiation of HSCs into Endothelial-Like Cells, and MSCs into Osteoblast-Like Cells

Upon confluence, hHSCs are transferred to fibronectin-coated 24, 12, and 6-well tissue culture dishes consecutively and finally to Petri dishes. HSC-derived endothelial-like cells will continue to be expanded. Preliminary data show that these cells display endothelial cell morphology, and express several endothelial cell markers (see e.g., FIG. 3 above). In addition, hHSC-derived endothelial cells express significantly more von Willebrand factor (vWF), an endothelial cell marker, than control cells (see e.g., FIG. 3 above). Adherent cells to fibronectin are differentiated with endothelial cell differentiation supplements (ECS), which include VEGF (10 ng/mL), bFGF (1 ng/mL), and IGF-1 (2 ng/mL), to HSC growth medium with 10% FBS. MSCs are differentiated into osteoblast-like cells per previous methods, with osteogenic stimulating supplements containing 100 nM dexamethasone, 50 μg/ml ascorbic acid and 100 mM β-glycerophosphate (see e.g., Alhadlaq and Mao, 2003; Alhadlaq et al., 2004; Alhadlaq et al., 2005; Stosich and Mao, 2005; Marion et al., 2005; Yourek et al., 2005; Moioli et al., 2006; Marion and Mao, 2006).

### Example 11

### Fabrication of PLGA Microspheres and Encapsulation of PDGF

These procedures follow studies described above and also those in Moioli et al. (2006). PLGA is a biocompatible and biodegradable synthetic copolymer of poly(L-lactic acid) and poly(glycolic acid), and has been widely used (see e.g., Lu et al., 2000; Shea et al., 2000; Burdick et al., 2001; Hedberg et al., 2003; Karp et al., 2003a; Ochi et al., 2003; Moioli et al., 2006). A total of 250 mg of poly(L-lactic acid) and poly(glycolic acid) (PLGA: 50:50, PLA:PGA) (Sigma, St Louis, Mo.) are dissolved in 1 mL dichloromethane. PDGF is encapsulated by PLGA microspheres by double emulsion technique as in our previous work (Moioli et al., 2006). The mixture is vortexed for 1 min. After adding 2 ml 1% PVA, mixture is vortexed for another 1 min. The resulting emulsion is added to 100 ml 0.1% PVA solution. The mixture of PVA/microsphere is added to 100 ml 2% isopropanol to remove dichloromethane, and to harden microspheres, and is continuously stirred under chemical fumehood for 2 hours. PDGF microspheres are collected by filtration and subsequently freeze-dried, and then dissolved into chloroform for 4 hrs, followed by vigorous shaking for 2 minutes. After clarifying for 4 hrs, the concentration of PDGF encapsulated per unit of microspheres is quantified using a PDGF ELISA kit (R&D Systems, St. Louis, Mo.) based on the product protocol. Microspheres encapsulating PDGF with predefined doses are suspended in 10 μl PBS. After cell seeding, PDGF encapsulated PLGA microspheres are injected into Matrigel solution by a microtip prior to implantation.

### Example 12

### Perfusion of Cell-Seeded Constructs

In case of poor cell survival in Matrigel infused βTCP constructs, mass transport can be enhanced by perfusion bioreactors developed in previous work (Vunjak-Novakovic et al., 1999; 2002). Briefly, perfusion of medium is established at a linear velocity through the scaffold in the range 10-100 μm, corresponding to the perfusion rates in native bone. In each pass, medium is equilibrated with respect to oxygen and pH in an external loop gas exchanger. Medium is replaced at a rate of 50% every other day. Perfusion time is optimized as a function of the outcome of engineered vascular bone as outlined in Table 3 above.

### Example 13

### Creation of Full-Thickness Calvarial Defects, Scaffolds and Surgical Implantation of Engineered Constructs

Eleven-wk-old nude rats are anesthetized by intraperitoneal injection (IP) of a cocktail containing 90% ketamine (100 mg/ml; Aveco, Fort Dodge, Iowa) and 10% Xylazine (20 mg/ml; Mobay, Shawnee, Kans.). Povidone Iodine (10%) is used to disinfect surgical areas. A 3 cm-long linear cut is made along the sagittal midline of the skull. Subcutaneous tissue and periosteum are deflected to expose the cortical bone surface. A full-thickness calvarial defect (5×1 mm³: 5 mm dia.) is created in the center of the parietal bone using a sterile dental bur with irrigation of phosphate buffered saline, following previously used methods (see e.g., Hong and Mao, 2004; Moioli et al., 2006). Per previous experience, this 5 mm diameter, full-thickness calvarial defect constitutes a critical defect, which fails to heal without bone grafting (see e.g., Hong and Mao, 2004; Moioli et al., 2006). Dura mater and adjacent cranial sutures are kept intact (Kopher and Mao, 2003; Hong and Mao, 2004; Moioli et al., 2006). HSCs or HSC-derived endothelial cells are seeded in the aqueous phase of Matrigel in a light vacuum at 4° C., as in studies described above. Matrigel is a basement membrane polymeric hydrogel that has been widely used for endothelial cell adhesion and angiogenesis experiments (see e.g., Abilez et al., 2006; Baker et al., 2006; Bruno et al., 2006; Mondrinos et al., 2006; Rajashekhar et al., 2006). Cell-Matrigel solution is infused into the pores of βTCP disks that have been seeded with hMSC-derived osteoblasts, followed by gelation of the Matrigel at 37° C. βTCP is obtained commercially with pore sizes between 200 to 400 μm (BD BioScience, San Diego, Calif.). Engineered tissue constructs with βTCP scaffold will fit into the 5 mm diameter, full-thickness calvarial defect, followed by the closure of the surgical flaps consisting of periosteum, subcutaneous soft tissue, and skin with 4-0 plain gut absorbable surgical suture.

### Example 14

### Tissue Harvest, Histology and Immunohistochemistry

Harvested calvarial specimens containing engineered bone are used for both demineralized preparations for paraffin embedding and un-demineralized embedding in plastic for quantitative bone histomorphometry with double-florescent labels (calcein and alizarin) of newly formed bone (see below). For demineralized preparations, specimens are fixed in 10% paraformaldehyde, demineralized in equal volumes of 20% sodium citrate and 50% formic acid, embedded in paraffin, and sectioned in the transverse plane at 10 μm thickness using standard histological procedures as in studies described above (cf., Mao et al., 1998; Wang and Mao, 2002; Kopher et al., 2003). Sequential sections are stained with hematoxylin and eosin, von Kossa, and Masson's trichrome stain for visualizing various regions of engineered bone. Undemineralized preparations are as described below. Immunohistochemistry of osteogenic and angiogenic markers follows previously developed methods (see e.g., Alhadlaq and Mao, 2005; Stosich et al., 2006; Sundaramurthy and Mao, 2006).

### Example 15

### Quantification of Bone Geometry by Computerized Histomorphometry

The engineered bone is quantitatively assessed by computerized histomorphometric analysis (ImagePro and Nikon Eclipse E800, Nikon Corp., Melville, N.Y.). Standardized grids (1175×880 μm²) are constructed and laid over histologic specimens under a 4× objective so that the engineered bone can be quantified. Numerical data are subjected to statistical analyses as described in each example.

### Example 16

### Quantification of Newly Formed Calvarial Bone by Double-Florescence Labeling and Computer-Assisted Dynamic Bone Histomorphometry

Calcein green (15 mg/kg) and alizarin red (20 mg/kg) injected i.p. two weeks and one week before sacrifice are visualized by computer-assisted dynamic bone histomorphometry (Parfitt et al., 1997; Mao, 2002; Kopher and Mao, 2003; Clark et al., 2005). Calvarial specimens are trimmed and dehydrated in graded ethanol and acetone, and further prepared for undecalcified embedding using 85% methyl methacrylate (MMA) and 15% dibutyl phthalate. The polymerized MMA-specimen blocks are trimmed with a band saw. Sequential undemineralized 15-μm sections are cut in the parasagittal plane using a Leica polycut microtome capable of cutting undemineralized calcified tissue specimens. Newly mineralized bone labeled with calcein in undemineralized sections is imaged under a fluorescence microscope (Mao, 2002; Kopher and Mao, 2003; Clark et al., 2005). Mineral apposition rate (MAR) is calculated by measuring the average distance between the subsequent calcein and alizarin labels divided by the time interval between the injection labels (7 days) (Clark et al., 2005). Bone formation rate (BFR) is defined as Bone formation rate (BFR/BS) was defined as MAR×BSf/BS (Clark et al., 2005). Numerical data are subjected to statistical analyses described in each example.

### Example 17

### Microindentation of Engineered Bone with Atomic Force Microscopy

The mechanical properties of engineered bone are tested by established method using atomic force microscopy (AFM) (see e.g., Hu et al., 2001; Patel and Mao, 2003; Radhakrishnan et al., 2003; Allen and Mao, 2004; Tomkoria et al., 2004; Clark et al., 2005). Mechanical testing with AFM is advantageous over macromechanical testing because the latter cannot distinguish separate mechanical properties of engineered bone. The sample is rapidly dried and glued onto a glass slide using fast-drying cyanoacrylate. Using a two-sided adhesive tape, the glass slide is fixed to AFM's mounting stainless steel disc, which is then magnetically mounted onto the piezoscanner of AFM. The sample is constantly irrigated with phosphate-buffered saline during AFM microindentation. Cantilevers with a nominal force constant of k=0.12 N/m and oxide-sharpened Si₃N₄ tips are used to apply microindentation against the newly harvested construct surface. Force spectroscopy data are obtained by driving the cantilever tip in the Z plane. Force mapping, involving data acquisition of microindentation load and the corresponding displacement in the Z plane during both extension and retraction of the cantilever tip, are recorded. Young's modulus (E) is then calculated from force spectroscopy data by following the Hertz model, which defines a relationship between contact radius, the microindentation load, and the central displacement:

E=3F(1−ν)/4√Rδ^{3/2}

Where E is the Young's modulus, F is the applied nanomechanical load, ν is the Poisson's ratio for a given region, R is the radius of the curvature of the AFM tip, and S is the amount of indentation. Young's modulus values of constructs from all groups are determined and compared to previously obtained similar values for native trabecular bone. The average Young's modulus of different locations are subjected to statistical analyses to indicate separate their mechanical properties.

### Example 18

### Mechanical Testing of Compressive and Shear Properties of Engineered Bone with Biaxial MTS Mechanical Testing Device

Engineered bone is harvested en bloc. The harvested samples are washed with PBS solution, blotted thoroughly to remove excess water, and potted using dental plaster (Lab Buff, Miles Dental Products, South Bend, Ind.) to secure the specimens in the testing apparatus (MTS 858 Mini Bionix II Machine, MTS Corp., Minneapolis, Minn.). Specimens are loaded in compression at an initial loading rate of 0.1 mm/s. Force (N) versus displacement (mm) is measured, and the modulus of elasticity, E (KPa), is calculated for each specimen. For shear testing, one of the potted lateral surrounding bone ends is attached to the loading axis, whilst leaving the other lateral portion attached to a fixed stage. An initial low displacement is applied to the moving axis (0.01 mm/s), displacing the moving side in respect to the fixed one. The resulting shear modulus is measured using Station Manager software. For both compressive and shear loading tests, different loading rates are investigated to determine the effects on loading rates on the outcome of mechanical testing, and if loading rates affect the outcome, the loading rate at the physical loading range of 1-4 Hz is used (Collins et al., 2004).

### Example 19

### Imaging of Engineered Bone with Digital X-Ray and MicroCT

Engineered bone is imaged with digital x-ray (Faxitron, Wheeling, Ill.) per our published approaches (Collins et al. 2005). Engineered bone is fixed in 10% formalin and imaged with multiple slices using a microcomputed tomography (pCT) system (ViVa CT 40, Scanco, Switzerland) at 21 μm resolution. Images are reconstructed for the 5×5×1 mm³ volume and threshold values are determined for each image based on the valley between the bone voxel and soft tissue voxel peaks from image histograms. The geometric width of engineered bone is quantified. All numerical values are subjected to ANOVA with Bonferroni tests. The adjacent native lamboidal bone will also be imaged by pCT to serve as controls for engineered bone. The analysis of pCT data for the native lamboidal bone is the same as engineered bone.

### Example 20

### Macrochannel and bFGF Promotion of Host Tissue Neovascularization

Experiments similar to those described in Example 3 were performed, but with a lower concentration of bFGF.

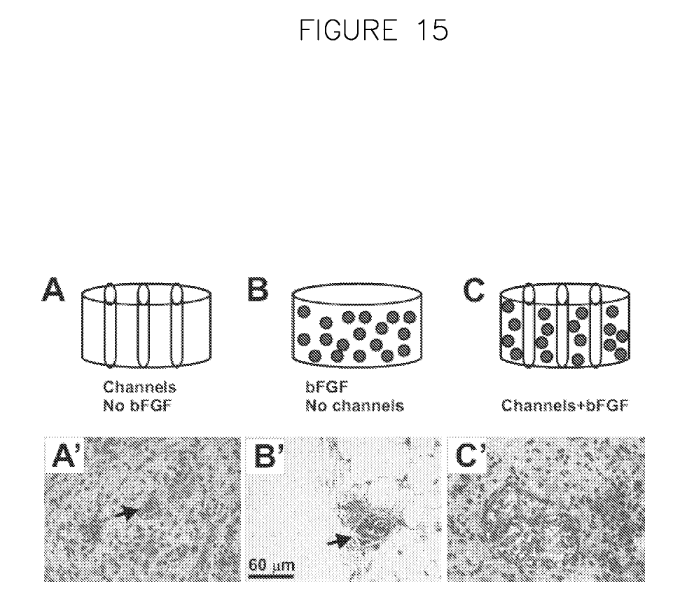

Poly(ethylene glycol) diacrylate (MW 3400; Nektar, Huntsville, Ala., USA) was dissolved in PBS (6.6% w/v) supplemented with 133 units/mL penicillin and 133 mg/mL streptomycin (Invitrogen, Carlsbad, Calif., USA). A photoinitiator, 2-hydroxy-1-[4-(hydroxyethoxy)phenyl]-2-methyl-1-propanone (Ciba, Tarrytown, N.Y., USA), was added at a concentration of 50 mg/mL. The resulting PEG cylinders were photopolymerized with UV light at 365 nm for 5 min (Glo-Mark, Upper Saddle River, N.J., USA). A total of 3 PEG hydrogel configurations were fabricated: 1) a total of 3 macrochannels (dia: 1 mm) were perforated in the photopolymerized PEG hydrogel (see e.g., FIG. 15A); 2) 0.5 μg/μL bFGF was loaded in PEG hydrogel without macrochannels (see e.g., FIG. 15B); and 3) a combination of 0.5 μg/μL bFGF and macrochannels (see e.g., FIG. 15C).

Male severe combined immune deficiency (SCID) mice (strain C.B17; 4-5 wk old) were anesthetized with intraperitoneal injection of ketamine (100 mg/kg) and xylazine (4 mg/kg). The mouse dorsum was clipped of hair and placed in a prone position, followed by disinfection with 10% povidone iodine and 70% alcohol. A 1 cm-long linear cut was made along the upper midsagittal line of the dorsum, followed by blunt dissection to create subcutaneous pouches. Each SCID mouse received 3 PEG hydrogel implants: PEG with macrochannels but without bFGF, bFGF-loaded PEG without macrochannels, or PEG with both bFGF and macrochannels. The incision was closed with absorbable plain gut 4-0 sutures. All PEG hydrogel cylinders were implanted in vivo for 4 wks.

Four weeks following subcutaneous implantation in the dorsum of SCID mice, PEG hydrogel samples were harvested. Following CO₂ asphyxiation, an incision was made aseptically in the dorsum of the SCID mouse. Following careful removal of the surrounding fibrous capsule, PEG hydrogel cylinders were isolated from the host, rinsed with PBS, and fixed in 10% formalin for 24 hrs. The PEG samples were then embedded in paraffin and sectioned in the transverse plane (transverse to macrochannels, c.f., FIG. 15A) at 5 μm thickness. Paraffin sections were stained with hematoxylin and eosin. Sequential adjacent sections were prepared for immunohistochemistry. Sections were deparaffinized, washed in PBS, and digested for 30 min at room temperature with bovine testicular hyaluronidase (1600 U/ml) in sodium acetate buffer at pH 5.5 with 150 mM sodium chloride. All immunohistochemistry procedures followed our previous methods (Mao et al., 1998; Alhadlaq and Mao, 2005; Sundaramurthy and Mao, 2006). Briefly, sections were treated with 5% bovine serum albumin (BSA) for 20 min at room temperature to block nonspecific reactions. The following antibodies were used: anti-vascular endothelial growth factor (anti-VEGF) (ABcam, Cambridge, Mass. USA), and biotin-labeled lectin from tritium vulgaris (wheat germ agglutinin) (WGA) with or without its inhibitor, actyleuraminic acid (Sigma, St. Louis, Mich., USA). WGA binds to carbohydrate groups of vascular endothelial cells rich in α-D-GlcNAc and NeuAc (Jinga et al., 2000; Izumi et al., 2003). After overnight incubation with primary antibodies in a humidity chamber, sections were rinsed with PBS and incubated with IgG antimouse secondary antibody (1:500; Antibodies Inc., Davis, Calif.) for 30 min. Sections were then incubated with streptavidin-HRP conjugate for 30 min in humidity chamber. After washing in PBS, the double linking procedure with the secondary antibody was repeated. Slides were developed with diaminobenzadine (DAB) solution and counterstained with Mayer's hematoxylin for 3 to 5 min. Counterstained slides were dehydrated in graded ethanol and cleared in xylene. The same procedures were performed for negative controls except for the omission of the primary antibodies.

Results showed that, upon 4-wk in vivo implantation in the dorsum of SCID mice, acellular PEG hydrogel with macrochannels but without bFGF demonstrated host tissue infiltration only in the lumen of macrochannels, but not in the rest of PEG (see e.g., FIG. 15A′). In contrast, acellular PEG hydrogel loaded with bFGF but without macrochannels demonstrated apparently random and isolated areas of host tissue infiltration (see e.g., FIG. 15B′). And PEG hydrogel with both macrochannels and bFGF demonstrated host tissue ingrowth in macrochannels, but not the rest of PEG (see e.g., FIG. 15C′). PEG hydrogel lacking both macrochannels and bFGF showed no host tissue infiltration (data not shown), consistent with previous data showing a lack of host tissue infiltration from host cells into PEG hydrogel (Alhadlaq et al., 2005; Stosich and Mao, 2005; 2006).

### Example 21

### Isolation and Culture-Expansion of Human Bone Marrow-Derived Mesenchymal Stem Cells (hMSCs)

Isolation and culture-expansion of human bone marrow-derived mesenchymal stem cells (hMSCs) was performed, consistent with procedures outlined in Example 9.

Human MSCs were isolated from fresh bone marrow samples of two anonymous adult donors (AllCells, Berkeley, Calif.), per previous methods (see e.g., Marion et al., 2005; Yourek et al., 2005; Moioli et al., 2006; Marion and Mao, 2006). After transferring bone marrow sample to a 50 mL tube, a total of 750 μL of RosetteSep was added (StemCell Technologies, Vancouver, Canada) and incubated for 20 min at room temperature. Then 15 mL of PBS in 2% FBS and 1 mM EDTA solution was added to the bone marrow sample to a total volume of approximately 30 ml. The bone marrow sample was then layered on 15 mL of Ficoll-Paque (StemCell Technologies) and centrifuged 25 min at 3000 g and room temperature. The entire layer of enriched cells was removed from Ficoll-Paque interface. The cocktail was centrifuged at 1000 rpm for 10 min. The solution was aspirated into 500 μL Dulbecco's Modified Eagle's Medium (Sigma-Aldrich Inc, St. Louis, Mo.) with 10% Fetal Bovine Serum (FBS) (Atlanta Biologicals, Lawrenceville, Ga.), and 1% antibiotic-antimycotic (Gibco, Carlsbad, Calif.), referred to as basal medium thereafter. The isolated mononuclear cells were counted, plated at approximately 0.5−1×10⁶ cells per 100-mm Petri dish and incubated in basal medium at 37° C. and 5% CO₂. After 24 hrs, non-adherent cells were discarded, whereas adherent mononuclear cells were washed twice with phosphate buffered saline (PBS) and incubated for 12 days with fresh medium change every other day (25). Upon 90% confluence, cells were removed from the plates using 0.25% trypsin and 1 mM EDTA for 5 min at 37° C., counted, and replated in 100-mm Petri dishes, referred to as Passage 1 cells.

### Example 22

### Differentiation of Human Mesenchymal Stem Cells into Adipogenic Cells

Second- and third-passage hMSCs were induced to differentiate into adipogenic cells by exposure to adipogenic medium consisting of basal medium supplemented with 0.5 μM dexamethasone, 0.5 μM isobutylmethylxanthine (IBMX), and 50 μM indomethacin, per prior methods (see e.g., Alhadlaq et al., 2005; Stosich and Mao, 2005, 2006; Marion and Mao, 2006). A subpopulation of hMSCs was continuously cultured in basal medium also in 95% air and 5% CO₂ at 37° C. with medium changes every other day. Oil-Red O staining (Sigma-Aldrich, St. Louis, Mo.) was used to verify adipogenesis (lipid formation). For in vitro assessment of adipogenic differentiation, hMSCs were treated with adipogenic medium for up to 5 wks. Monolayer cultured hMSCs with or without adipogenic differentiation were fixed in 10% formalin and subjected to Oil-Red O staining. The plates were examined under an inverted microscope at 10× magnification for the presence or absence of lipid vacuoles.

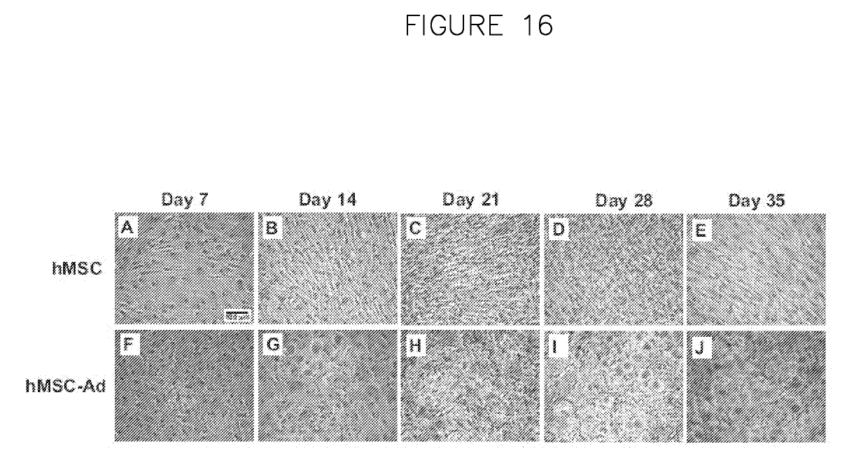

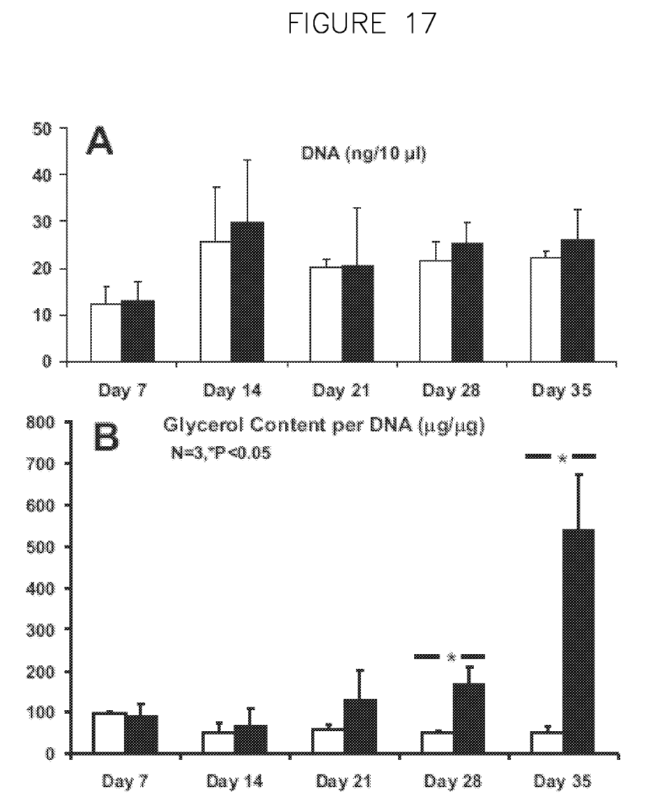

Results shoped that human mesenchymal stem cells were differentiated into adipogenic cells in vitro over the observed 35 days in ex vivo culture (see e.g., FIG. 16). In comparison with hMSCs without adipogenic differentiation (see e.g., FIG. 16A-17E), hMSC-derived adipogenic cells reacted positively to Oil-red O staining, and progressively so over the 35 day course (see e.g., FIG. 16F-17J). This is consistent with previous data showing the expression of PPAR-γ2 by hMSC-derived adipogenic cells following less than 2 wks of treatment in adipogenic medium (see e.g., Alhadlaq et al., 2005). The total DNA content of culture samples between hMSCs and hMSC-derived adipogenic cells lacked statistically significant differences over the observed 35 days (see e.g., FIG. 17A). However, glycerol contents of hMSC-derived adipogenic cell samples were significantly higher than those of hMSCs at 28 and 35 days in culture, suggesting that hMSC-derived adipogenic cells gradually accumulate intracellular lipid vacuoles in vitro.

### Example 23

### Encapsulation hMSC-Derived Adipogenic Cells in PEG Hydrogel and In Vivo Implantation

In a parallel experiment to utilize the model system above of macrochannels and bioactive factor in PEG hydrogel (see Example 3), hMSCs and hMSC-derived adipogenic cells were encapsulated to determine whether the engineered macrochannels and bFGF promoted vascularized adipogenesis.

PEG hydrogel was dissolved in sterile PBS supplemented with 100 U/ml penicillin and 100 μg/ml streptomycin (Gibco, Carlsbad, Calif.) to a final solution of 10% w/v. The photoinitiator, 2-hydroxy-1-[4-(hydroxyethoxy)phenyl]-2-methyl-1-propanone (Ciba, Tarrytown, N.Y.), was added to the PEGDA solution. After 1 wk of adipogenic differentiation or culture in basal medium, hMSCs or hMSC-derived adipogenic cells were removed from the culture plates with 0.25% trypsin and 1 mM EDTA for 5 min at 37° C., counted, and resuspended separately in PEG polymer/photoinitiator solutions at a density of 3×106 cells/mL. An aliquot of 75 μL cell/polymer/photoinitiator suspension was loaded into sterilized plastic caps of 0.075 mL microcentrifuge tubes (6×4 mm: dia.×height) (Fisher Scientific, Hampton, N.H.), followed by photo-polymerization with long-wave, 365-nm ultraviolet lamp (Glo-Mark, Upper Saddle River, N.J.) at an intensity of approximately 4 mW/cm² for 5 min. The photo-polymerized cell-PEG constructs were removed from the plastic caps and transferred into a 12 well plate in corresponding adipogenic medium. A total of 0.5 μg/μL bFGF was loaded in PEG hydrogel prior to photopolymerization. The creation of 3 macrochannels followed the approach as described above (see Example 20). Twelve weeks following subcutaneous implantation in the dorsum of athymic nude mice, PEG hydrogel cylinders were harvested. All tissue processing, histological and immunohistochemical procedures were the same as described above (see Example 20).

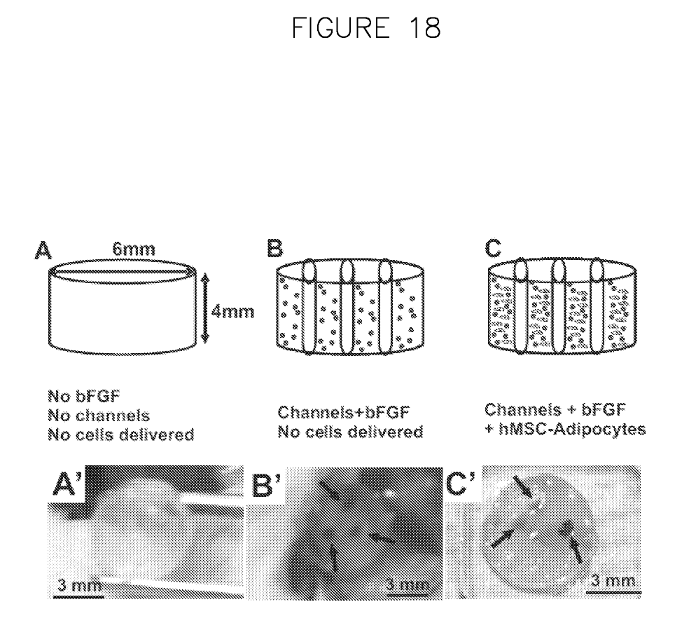

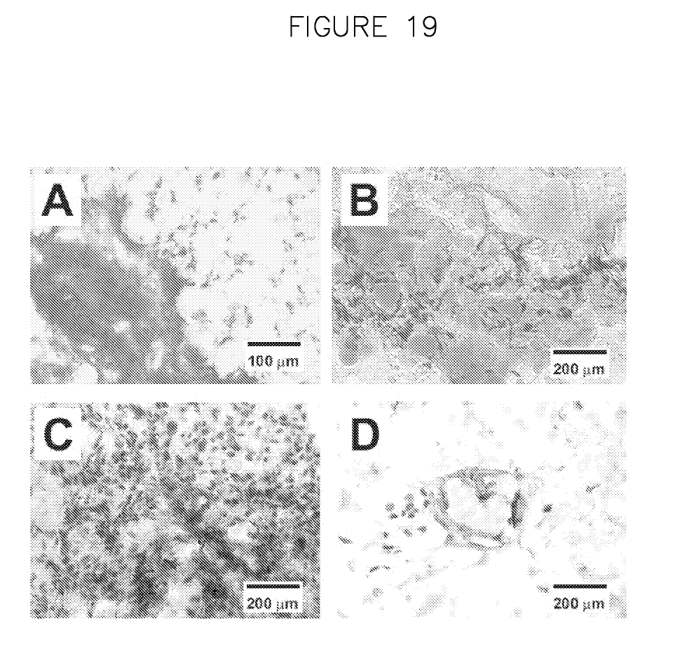

Results showed that PEG hydrogel was not permissive to cell infiltration (see e.g., FIG. 18A′). Such findings were consistent earlier studies (see e.g., Alhadlaq et al., 2005; Stosich and Mao, 2005; 2006). However, PEG hydrogel loaded with engineered macrochannels and bFGF showed not only darker color, but also 3 red circles in the transverse plane (see e.g., FIG. 18B′). Further, PEG hydrogel with both macrochannels and bFGF, and seeded with hMSC-derived adipogenic cells showed not only darker color, but also red circles (see e.g., FIG. 18C′). Upon histological and immunohistochemical examination, PEG hydrogel encapsulating hMSC-derived adipogenic cells with built-in macrochannels and bFGF showed islands of tissue formation (see e.g., FIG. 19A). Many islands of the engineered tissue were Oil-red O positive, shown as a representative in FIG. 19B, suggesting the presence of adipogenesis. Anti-VEGF antibody showed positive staining in the apparently interstitial tissue (see e.g., FIG. 19C), and anti-WGA lectin antibody was localized to the vicinity of engineered adipose tissue (see e.g., FIG. 19D), suggesting that engineered neovascularization promoted adipogenesis.

### Example 24

### Molecular Markers for Vascular Endothelial Cells

Vascular progenitor cells were analyzed for expression of vascular endothelial growth factors 2 or Flk 1, both molecular markers for vascular endothelial cells. Hematopoietic stem cell isolation, culture, differentiation, and labeling were performed consistent with that described in Example 2.

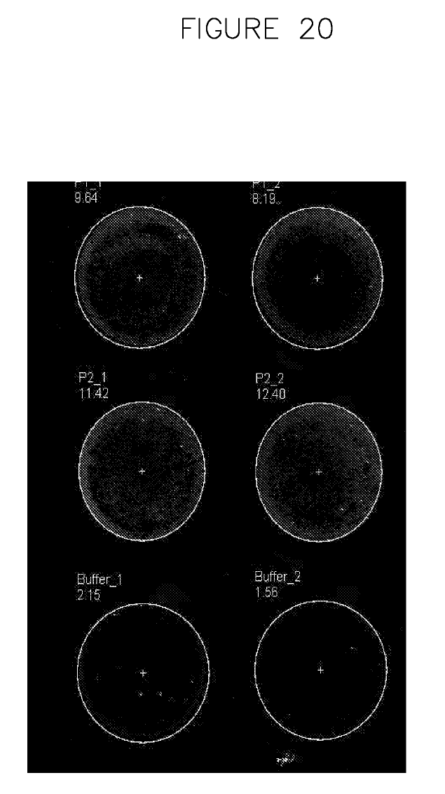

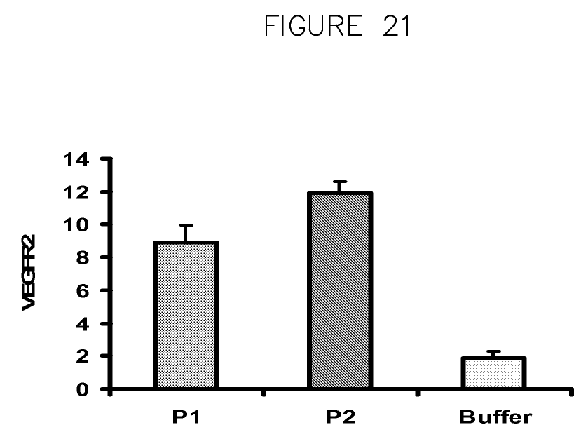

Results showed that vascular progenitor cells (Passage 1 cells in 1° ′ column and Passage 2 cells in the 2^{nd} column) were found to express both vascular endothelial growth factors 2 or Flk 1, in comparison with a lack of VEGF/Flk1 expression of the buffer sulocation (see e.g., FIG. 20). Quantification of VEGF2 content indicated that both P1 and P2 cells express significantly more VEGF2 than the buffer medium (see e.g., FIG. 21).

These data demonstrate that HSCs isolated from human bone marrow can differentiate into endothelial-like cells, as evidenced by expression of VEGF2 and Flk1, both endothelial cell markers.

### Example 25

### Cell Labeling Experiment

A porous βTCP scaffold seeded with both osteoprogenitor cells and vascular progenitor cells was analyzed for co-inhabitation of both cell types. Methods of scaffold infusion with progenitor cells were consistent with that described in Example 1.

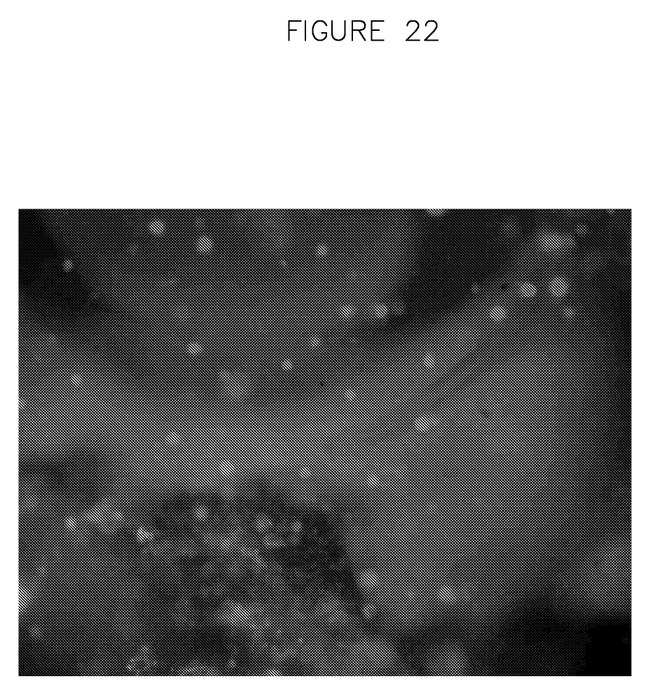

Results showed that osteoprogenitors labeled with green fluorescence protein (GFP) co-inhabited with vascular progenitor cells labeled with CM-DII in red, both in the porous βTCP scaffold (see e.g., FIG. 22). In vivo implantation of osteoprogenitor and vascular progenitor seeded βTCP scaffold yielded the formation of vascularized bone, as demonstrated above. (see e.g., Example 1; FIG. 2).

These data demonstrate that human osteoprogenitor cells and vascular progenitor cells co-seeded in different spatial regions of a biocompatible material can successfully differentiate into bone and vascular tissue, respectively, while co-inhabiting the scaffold.

### APPENDIX

### Literature Cited

Abilez O, Benharash P, Mehrotra M, Miyamoto E, Gale A, Picquet J, Xu C, Zarins C (2006) A novel culture system shows that stem cells can be grown in 3D and under physiologic pulsatile conditions for tissue engineering of vascular grafts. J Surg Res 132:170-178.
Alberts B, Johnson B, Lewis J, Raff M, Roberts K, Walter P (2002) Molecular Biology of the Cell. 4th Ed. New York, Garland Publishing. pp. 1183-1184.
Alhadlaq A, Elisseeff J H, Hong L, Williams C G, Caplan A I, Sharma B, Kopher R A, Tomkoria S, Lennon D P, Lopez A, Mao J J (2004) Adult stem cell driven genesis of human-shaped articular condyle. Ann Biomed Eng 32:911-923.
Alhadlaq A, Mao J J (2003) Engineered neogenesis of human-shaped mandibular condyle from rat mesenchymal stem cells. J Dent Res 82:951-956.
Alhadlaq A, Mao J J (2004) Mesenchymal stem cells: Isolation and therapeutics. Stem Cells Dev 13:436-448.
Alhadlaq A, Mao J J (2005) Osteochondral Tissue Engineering—Regeneration of Articular Condyle from Mesenchymal Stem Cells. In Ma P X, Elisseeff J H (Ed) Scaffolding for Tissue Engineering. Taylor & Francis, Boca Raton, Fla., pp. 545-564.
Alhadlaq A, Mao J J (2005) Tissue-engineered osteochondral constructs in the shape of an articular condyle. J Bone Joint Surg Am 87:936-944.
Alhadlaq, A. and J. J. Mao. Tissue-engineered neogenesis of human-shaped mandibular condyle from rat mesenchymal stem cells. J Dent Res 82:951-956, 2003.
Alhadlaq, A., and J. J. Mao. Mesenchymal stem cells: Isolation and therapeutics. Stem Cells Dev. 13: 436-448, 2004.
Alhadlaq, A., J. H. Elisseeff, L. Hong, C. G. Williams, A. I. Caplan, B. Sharma, R. A. Kopher, S. Tomkoria, D. P. Lennon, A. Lopez, J. J. Mao. Adult stem cell driven genesis of human-shaped articular condyle. Ann Biomed Eng 32:911-923, 2004.
Alhadlaq, A., M. H. Tang, J. J. Mao. Engineered adipose tissue from human mesenchymal stem cells maintains predefined shape and dimension: implications in soft tissue augmentation and reconstruction. Tissue Eng. 11:556-66, 2005.
Allen D M, Mao J J (2004) Heterogeneous nanostructural and nanoelastic properties of pericellular and interterritorial matrices of chondrocytes by atomic force microscopy. J Struct Biol 145:196-204.
Almubarak R, Dasilveira A, Mao J J (2005) Expression and mechanical modulation of matrix metalloproteinase 1 and 2 genes in facial and cranial sutures. Cell & Tiss Res 321:465-471.
Alsberg E, Anderson K W, Albeiruti A, Rowley J A, Mooney D J (2002) Engineering growing tissues. Proc Natl Acad Sci USA 17; 99:12025-12030.
Anusaksathien, O., and W. V. Giannobile. Growth factor delivery to reengineering periodontal tissues. Curr Pharm Biochnol 3: 129-139, 2002.
Arai F, Hirao A, Suda T (2005) Regulation of hematopoiesis and its interaction with stem cell niches. Int J Hematol 82:371-376.
Arzate, H., J. Chimal-Monroy, L. Hernandez-Lagunas, L. Diaz de Leon. Human cementum protein extract promotes chondrogenesis and mineralization in mesenchymal cells. J Periodontal Res 31:144-148, 1996.
Aubin J E (1998) Bone stem cells. J Cell Biochem Suppl 30-31:73-82.
Badylak S F, Park K, Peppas N, McCabe G, Yoder M (2001) Marrow-derived cells populate scaffolds composed of xenogeneic extracellular matrix. Exp Hematol 29:1310-1318.
Baker J H, Huxham L A, Kyle A H, Lam K K, Minchinton A I (2006) Vascular-specific quantification in an in vivo Matrigel chamber angiogenesis assay. Microvasc Res 71:69-75.
BarKana, I., A. S, Narayanan, A. Grosskop, N. Savion, S. Pitaru. Cementum attachment protein enriches putative cementoblastic populations on root surfaces in vitro. J Dent Res 79:1482-1488, 2000.
Batra R K, Sharma S, Dubinett S M (2000) New gene and cell-based therapies for lung cancer. Semin Respir Crit. Care Med 21:463-472.
Benoit D S, Anseth K S (2005) The effect on osteoblast function of colocalized RGD and PHSRN epitopes on PEG surfaces. Biomaterials 26:5209-5220.
Bianco P, Riminucci M, Gronthos S, Robey P G (2001) Bone marrow stromal stem cells: nature, biology, and potential applications. Stem Cells 19:180-192.
Boabaid, F., C. W. Gibson, M. A. Kuehl, J. E. Berry, M. L. Snead, F. H. Nociti, E. Katchburian, M. J. Somerman. Leucine-rich amelogenin peptide: a candidate signaling molecule during cementogenesis. J Periodontol 75: 1126-1136, 2004.
Bohnsack B L, Hirschi K K (2004) Red Light, Green Light: Signals That Control Endothelial Cell Proliferation during Embryonic Vascular Development. 12:1506-1511.
Bollerot K, Pouget C, Jaffredo T (2005) The embryonic origins of hematopoietic stem cells: a tale of hemangioblast and hemogenic endothelium. APMIS 113:790-803.
Boyd F T, Cheifetz S, Andres J, Laiho M, Massague J (1990) Transforming growth factor-beta receptors and binding proteoglycans. J Cell Sci Suppl 13:131-138.
Bruder S P, Jaiswal N, Ricalton N S, Mosca J D, Kraus K H, Kadiyala S (1998). Mesenchymal stem cells in osteobiology and applied bone regeneration. Clin Orthop 355 Suppl:S247-S256.
Bruno S, Bussolati B, Scacciatella P, Marra S, Sanavio F, Tarella C, Camussi G (2006) Combined administration of G-CSF and GM-CSF stimulates monocyte-derived pro-angiogenic cells in patients with acute myocardial infarction. Cytokine 34:56-65.
Burdick J A, Mason M N, Anseth K S (2001). In situ forming lactic acid based orthopaedic biomaterials: influence of oligomer chemistry on osteoblast attachment and function. J Biomater Sci Polym Ed 12:1253-1265.
Burdick J A, Mason M N, Hinman A D, Thome K, Anseth K S (2002) Delivery of osteoinductive growth factors from degradable PEG hydrogels influences osteoblast differentiation and mineralization. J Control Rel 83:53-63.
Caffesse, R. G., M. de la Rosa, L. F. Mota. Regeneration of soft and hard tissue periodontal defects. Am J Dent 15:339-345, 2002.
Caplan A I (1994) The mesengenic process. Clin Plast Surg 21:429-435.
Caplan A I (2005) Review: mesenchymal stem cells: cell-based reconstructive therapy in orthopedics. Tissue Eng 11:1198-1211.
Caplan, A. I. Mesenchymal stem cells. J Orthop Res 9:641-650, 1991.
Carter D R, Beaupre G S, Giori N J, Helms J A (1998) Mechanobiology of skeletal regeneration. Clin Orthop 355 Suppl:S41-S55.
Caterson E J, Nesti U, Albert T, Danielson K, Tuan R (2001) Application of mesenchymal stem cells in the regeneration of musculoskeletal tissues. Med Gen Med. 5:E1.
Chen G, Ushida T, Tateishi T (2001). Poly(DL-lactic-co-glycolic acid) sponge hybridized with collagen microsponges and deposited apatite particulates. J Biomed Mater Res 57:8-14.
Cho S W, Kim I, Kim S H, Rhie J W, Choi C Y, Kim B S. Enhancement of adipose tissue formation by implantation of adipogenic-differentiated preadipocytes. Biochem Biophys Res Commun. 2006 Jun. 30; 345(2):588-94.
Choi S H, Park T G (2002) Synthesis and characterization of elastic PLGA/PCL/PLGA tri-block copolymers. J Biomater Sci Polym Ed 13:1163-1173.
Chowdhury S, Thomas V, Dean D, Catledge S A, Vohra Y K (2005) Nanoindentation on porous bioceramic scaffolds for bone tissue engineering. J Nanosci Nanotechnol 5:1816-1820.
Clark P A, Clark A C, Hu K, Mao J J (2006) Nanomechanical and micromechanical manipulation of bone-implant interface. Materials Science and Engineering C Special Issue on Nanostructured Materials for Biomedical Applications. (In press).
Clark P A, Sumner D R, Clark A M, Mao J J (2005) Modulation of bone ingrowth of rabbit femur titanium implants by in vivo axial micromechanical stresses. J App Physiol 98:1922-1929.
Collier J H, Camp J P, Hudson T W, Schmidt C E (2000) Synthesis and characterization of polypyrrole-hyaluronic acid composite biomaterials for tissue engineering applications. J Biomed Mater Res 50:574-584.
Collins J M, Ramamoorthy K, Da Silveira A, Patston, P A, Mao J J (2005) Microstrain in intramembranous bones induces altered gene expression of MMP1 and MMP2 in the rat. J Biomech 38:485-492.
Colnot C I, Helms J A (2001) A molecular analysis of matrix remodeling and angiogenesis during long bone development. Mech Dev 100:245-250.
Correia A S, Anisimov S V, Li J Y, Brundin P (2005) Stem cell-based therapy for Parkinson's disease. Ann Med 37:487-498.
Deng M J, Jin Y, Shi J N, Lu H B, Liu Y, He D W, Nie X, Smith A J (2004) Multilineage differentiation of ectomesenchymal cells isolated from the first branchial arch. Tissue Eng 10:1597-1606.
Ebihara Y, Masuya M, Larue A C, Fleming P A, Visconti R P, Minamiguchi H, Drake C J, Ogawa M. (2006) Hematopoietic origins of fibroblasts: II. In vitro studies of fibroblasts, CFU-F, and fibrocytes. Exp Hematol 34:219-229.
Ehrbar M, Djonov V G, Schnell C, Tschanz S A, Martiny-Baron G, Schenk U, Wood J, Burri P H, Hubbell J A, Zisch A H (2004) Cell-demanded liberation of VEGF121 from fibrin implants induces local and controlled blood vessel growth. Circ Res 94:1124-1132.
Einhorn T A (1998). The cell and molecular biology of fracture healing. Clin Orthop 355 Suppl:S7-S21.
Einhorn T A (2005) The science of fracture healing. J Orthop Trauma 19(10 Suppl):S4-S6.
Evans D J, Noden D M (2006) Spatial relations between avian craniofacial neural crest and paraxial mesoderm cells. Dev Dyn 235:1310-1325.
Ferrara N (2005) VEGF as a therapeutic target in cancer. Oncology 69 Suppl 3:11-16.
Feve B. (2005) Adipogenesis: cellular and molecular aspects. Best Pract Res Clin Endocrinol Metab. 2005 December; 19(4):483-99.
Freed, L. E., A. P. Hollander, I. Martin, J. R. Barry, R. Langer, G. Vunjak-Novakovic. Chondrogenesis in a cell-polymer-bioreactor system. Exp Cell Res 240:58-65, 1998.
Freed, L. E., G. Vunjak-Novakovic, R. Langer. Cultivation of cell-polymer cartilage implants in bioreactors. J Cell Biochem 51:257-264, 1993.
Friedenstein A J, Deriglasova U F, Kulagina N N, Panasuk A F, Rudakowa S F, Luria E A, Ruadkow I A (1974) Precursors for fibroblasts in different populations of hematopoietic cells as detected by the in vitro colony assay method. Exp Hematol 2:83-92.
Fukuchi Y, Nakajima H, Sugiyama D, Hirose I, Kitamura T, Tsuji K (2004) Human placenta-derived cells have mesenchymal stem/progenitor cell potential. Stem Cells 22:649-658.
Gao, J., J. E. Dennis, L. A. Solchaga, A. S. Awadallah, V. M. Goldberg, A. I. Caplan. Tissue-engineered fabrication of an osteochondral composite graft using rat bone marrow-derived mesenchymal stem cells. Tissue Engineering 7:363-371, 2001.
Gestrelius, S., C. Andersson, D. Lidstrom, L. Hammarstrom, M. Somerman. In vitro studies on periodontal ligament cells and enamel matrix derivative. J Clin Periodontol 24:685-692, 1997.
Gimble J, Guilak F (2003) Adipose-derived adult stem cells: isolation, characterization, and differentiation potential. Cytotherapy 5:362-369.
Glaser R L, Jiang W, Boyadjiev S A, Tran A K, Zachary A A, Van Maldergem L, Johnson D, Walsh S, Oldridge M, Wall S A, Wilkie A O, Jabs E W (2000). Paternal origin of FGFR2 mutations in sporadic cases of Crouzon syndrome and Pfeiffer syndrome. Am J Hum Genet. 66:768-777.
Goldberg V M, Caplan A I (1994). Biological resurfacing: an alternative to total joint arthroplasty. Orthopedics 17:819-821.
Goldstein S A (2002). Tissue engineering: functional assessment and clinical outcome. Ann N Y Acad Sci 961:183-192.
Grainger D W (2004) Controlled-release and local delivery of therapeutic antibodies. Expert Opin Biol Ther 4:1029-1044.
Grau N, Daw J, Patel R V, Lewis N R, Mao J J (2005) Nanomechanical and nanostructural properties of synostosed human cranial sutures. J Craniofac Surg 16:789-794.
Griffith L G, Naughton G (2002) Tissue engineering—current challenges and expanding opportunities. Science 295:1009-1014.
Grunewald M, Avraham I, Dor Y, Bachar-Lustig E, Itin A, Yung S, Chimenti S, Landsman L, Abramovitch R, Keshet E (2006) VEGF-induced adult neovascularization: recruitment, retention, and role of accessory cells. Cell 124:175-189.
Guldberg R E, Oest M, Lin A S, Ito H, Chao X, Gromov K, Goater J J, Koefoed M, Schwarz E M, O. Keefe R J, Zhang X (2004) Functional integration of tissue-engineered bone constructs. J Musculoskelet Neuronal Interact 4:399-400.
Guo X E (2000) Mechanical Properties of Cortical Bone and Cancellous Bone Tissue, in Bone Mechanics Handbook, 2nd Edition, Edi: Cowin S C, CRC Press. Pp. 66-74.
Guo X E, Kim C H (2002) Mechanical consequence of bone loss and treatment in trabecular bone: A 3D microstructural model. Bone 30:404-411.
Harel S, Watanabe K, Linke I, Schain R J (1972). Growth and development of the rabbit brain. Biol Neonate 1:381-399.
Harris W H, Crothers O D, Moyen B J, Bourne R B (1978). Topical hemostatic agents for bone bleeding in humans. A quantitative comparison of gelatin paste, gelatin sponge plus bovine thrombin, and microfibrillar collagen. J Bone Joint Surg Am 60:454-456.
Hedberg E L, Tang A, Crowther R S, Carney D H, Mikos A G (2003). Controlled release of an osteogenic peptide from injectable biodegradable polymeric composites. J Control Release 84:137-150.
Hee C K, Jonikas M A, Nicoll S B (2006) Influence of three-dimensional scaffold on the expression of osteogenic differentiation markers by human dermal fibroblasts. Biomaterials 27:875-884.
Helms J A, Cordero D, Tapadia M D (2005) New insights into craniofacial morphogenesis. Development 132:851-861.
Hiraoka Y, Yamashiro H, Yasuda K, Kimura Y, Inamoto T, Tabata Y. In situ regeneration of adipose tissue in rat fat pad by combining a collagen scaffold with gelatin microspheres containing basic fibroblast growth factor. Tissue Eng. 2006 June; 12(6):1475-87,
Ho M M, Kelly T N, Guo X E, Ateshian G A, Hung C T (2006) Spatially varying material properties of the rat caudal intervertebral disc. Spine (In press).
Hollinger J O, Seyfer A E (1994) Bioactive factors and biosynthetic materials in bone grafting. Clin Plast Surg 21:415-418.
Hong L, Mao J J (2004) A engineered cranial suture from autologous cells and BMP2. Journal of Dental Research 83:751-756.
Hori, Y., T. Nakamura, K. Matsumoto, Y. Kurokawa, S. Satomi, Y. Shimizu. Experimental study on in situ tissue engineering of the stomach by an acellular collagen sponge scaffold graft. ASAIO J 47: 206, 2001.
Hu K, Radhakrishnan P, Patel R V, Mao J J (2001). Regional structural and viscoelastic properties of fibrocartilage upon dynamic nanoindentation of the articular condyle. J Struct Biol 136:470-475.
Ikezawa, K., C. E. Hart, D. C. Williams, A. S, Narayanan. Characterization of a cementum derived growth factor as an insulin-like growth factor-I like molecule. Connect Tissue Res 36:309-319, 1997.
Ishii M, Merrill A E, Chan Y S, Gitelman I, Rice D P, Sucov H M, Maxson R E Jr (2003) Msx2 and Twist cooperatively control the development of the neural crest-derived skeletogenic mesenchyme of the murine skull vault. Development 130:6131-6142.
Jen A, Madorin K, Vosbeck K, Arvinte T, Merkle H P (2002). Transforming growth factor beta-3 crystals as reservoirs for slow release of active TGF-beta3. J Control Release. 17; 78(1-3):25-34.
Jiang X, Iseki S, Maxson R E, Sucov H M, Morriss-Kay G M (2002). Tissue origins and interactions in the mammalian skull vault. Dev Biol 241:106-116.
Jin H, Aiyer A, Su J, Borgstrom P, Stupack D, Friedlander M, Vamer J (2006) A homing mechanism for bone marrow-derived progenitor cell recruitment to the neovasculature. J Clin Invest 116:652-662.
Jones E A, Kinsey S E, English A, Jones R A, Straszynski L, Meredith D M, Markham A F, Jack A, Emery P, McGonagle D (2002). Isolation and characterization of bone marrow multipotential mesenchymal progenitor cells. Arthritis Rheum. 46(12):3349-3360.
Kafri T, Blomer U, Peterson D A, Gage F H, Verma I M (1997) Sustained expression of genes delivered directly into liver and muscle by lentiviral vectors. Nature Genet. 17:314-317.
Kaigler D, Krebsbach P H, Polyerini P J, Mooney D J (2003) Role of vascular endothelial growth factor in bone marrow stromal cell modulation of endothelial cells. Tissue Eng 9:95-103.
Kamminga L M, de Haan G (2006) Cellular memory and hematopoietic stem cell aging. Stem Cells 24:1143-1149.
Kan S H, Elanko N, Johnson D, Comejo-Roldan L, Cook J, Reich E W, Tomkins S, Verloes A, Twigg S R, Rannan-Eliya S, McDonald-McGinn D M, Zackai E H, Wall S A, Muenke M, Wilkie A O (2002). Genomic screening of fibroblast growth-factor receptor 2 reveals a wide spectrum of mutations in patients with syndromic craniosynostosis. Am J Hum Genet. 70:472-486.
Kao, R. T., G. Conte, D. Nishimine, S. Dault. Tissue engineering for periodontal regeneration. J Calif Dent Assoc 33: 205-215, 2005.
Karp J M, Rzeszutek K, Shoichet M S, Davies J E (2003b) Fabrication of precise cylindrical three-dimensional tissue engineering scaffolds for in vitro and in vivo bone engineering applications. J Craniofac Surg 14:317-323.
Karp J M, Shoichet M S, Davies J E (2003a) Bone formation on two-dimensional poly(DL-lactide-co-glycolide) (PLGA) films and three-dimensional PLGA tissue engineering scaffolds in vitro. J Biomed Mater Res 64A:388-396.
Kawamura, S., S. Wakitani, T. Kimura, A. Maeda, A. I. Caplan, K. Shino, T. Ochi. Articular cartilage repair. Rabbit experiments with a collagen gelbiomatrix and chondrocytes cultured in it. Acta Orthop Scand 69: 56-62, 1998.
Kelly J L, Findlay M W, Knight K R, Penington A, Thompson E W, Messina A, Morrison W A. Contact with Existing Adipose Tissue is Inductive for Adipogenesis in Matrigel. Tissue Eng. 2006 (In press).
King, G. N., D. L. Cochran. Factors that modulate the effects of bone morphogenetic protein-induced periodontal regeneration: a critical review. J Periodontol 73:925-936, 2002.
King, G. N., F. J. Hughes. Bone morphogenic protein-2 stimulates cell recruitment and cementogenesis during early wound healing. J Clin periodontal 28:465-475, 2001.
Knez P, Nelson K, Hakimi M, Al-Haidary J, Schneider C, Schmitz-Rixen T (2004) Rotational in vitro compliance measurement of diverse anastomotic configurations: a tool for anastomotic engineering. J Biomech 37:275-280.
Koenig A L, Gambillara V, Grainger D W (2003) Correlating fibronectin adsorption with endothelial cell adhesion and signaling on polymer substrates. J Biomed Mater Res A 64:20-37.
Koepp H E, Schorlemmer S, Kessler S, Brenner R E, Claes L, Gunther K P, Ignatius A A (2004) Biocompatibility and osseointegration of beta-TCP: histomorphological and biomechanical studies in a weight-bearing sheep model. J Biomed Mater Res B Appl Biomater 70:209-217.
Kopher R A, Mao J J (2003) Sutural growth modulated by the oscillatory component of micromechanical strain. J Bone Miner Res 25:107-113.
Kopher R A, Nudera J A, Wang X, O'Grady K, Mao J J (2003) Expression of in vivo mechanical strain upon different wave forms of exogenous forces in rabbit craniofacial sutures. Ann Biomed Eng 31:1125-1131.
Krebsbach P H, Kuznetsov S A, Bianco P, Robey P G (1999). Bone marrow stromal cells: characterization and clinical application. Crit. Rev Oral Biol Med 10:165-181.
Krebsbach P H, Kuznetsov S A, Satomura K, Emmons R V, Rowe D W, Robey P G (1997) Bone formation in vivo: comparison of osteogenesis by transplanted mouse and human marrow stromal fibroblasts. Transplantation 63:1059-1069.
Krebsbach P H, Robey P G (2002) Dental and skeletal stem cells: potential cellular therapeutics for craniofacial regeneration. J Dent Educ 66:766-773.
Krebsbach, P. H., S. A. Kuznetsov, K. Satomura, R. V. Emmons, D. W. Rowe, P. G. Robey. Bone formation in vitro: comparison of osteogenesis by transplanted mouse and human marrow stromal fibroblasts. Transplantation 63: 1059-1069, 1997.
Kuboki, Y., M. Sasaki, A. Sato, H. Takita, H. Kato. Regeneration of periodontal ligament and cementum by BMP-applied tissue engineering. Eur J Oral Sci 106:197-203, 1998.
Kuznetsov, S. A., P. H. Krebsbach, K. Satomura, J. Kerr, M. Riminucci, D. Benayahu, P. G. Robey. Single-colony derived strains of human marrow stromal fibroblasts form bone after transplantation in vivo. J Bone and Mineral Res 12:1335-1347, 1997.
Lammi M, Tammi M (1998). Densitometric assay of nanogram quantities of proteoglycans precipitated on nitrocellulose membrane with safranin O. Analytical Biochem 168:352-357.
Landesberg R, Takeuchi E, Puzas J E (1999) Signal transduction by cytokines in temporomandibular joint disc cells. Arch Oral Biol 44:41-49.
Langer R, Vacanti J P (1993) Tissue engineering. Science 260(5110):920-926.
Langer R S, Vacanti J P (1999) Tissue engineering: the challenges ahead. Sci Am 280:86-89.
Langer, R., J. P. Vacanti. Tissue engineering. Science 260:920-926, 1993.
Lee K Y, Mooney D J (2001) Hydrogels for tissue engineering. Chem Rev 101:1,869-1,879.
Lennon D P, Haynesworth S E, Young R G, Dennis J E, Caplan A I (1995). A chemically defined medium supports in vitro proliferation and maintains the osteochondral potential of rat marrow-derived mesenchymal stem cells. Exp Cell Res 219:211-222.
Leung, K. S., L. Qin, L. K. Fu, C. W. Chan. A comparative study of bone to bone repair and bone to tendon healing in patella-patella tendon complex in rabbits. Clinical Biomechanics 17:594-602, 2002.
Levenberg S (2005) Engineering blood vessels from stem cells: recent advances and applications. Curr Opin Biotechnol 16:516-523.
Li W, Johnson S A, Shelley W C, Yoder M C (2004) Hematopoietic stem cell repopulating ability can be maintained in vitro by some primary endothelial cells. Exp Hematol 32:1226-1237.
Lieb, E., J. Tessmar, M. Hacker, C. Fischbach, D. Rose, T. Slunk, A. G. Mikos, A. Gopferich, M. B. Schulz. Poly(D,L-lactic acid)-poly(ethylene glycol)-monomethyl ether diblock copolymers control adhesion and osteoblastic differentiation of marrow stromal cells. Tissue Engineering 9:71-84, 2003.
Lo, H., S. Kadiyala, S. E. Guggino, K. W. Leong. Poly(L-lactic acid) foams with cell seeding and controlled-release capacity. J Biomed Mater Res 30:475-484, 1996.
Lu L, Peter S J, Lyman M D, Lai H L, Leite S M, Tamada J A, Uyama S, Vacanti J P, Langer R, Mikos A G (2000) In vitro and in vivo degradation of porous poly(DL-lactic-co-glycolic acid) foams. Biomaterials 21:1837-1845.
Luo Y, Shoichet M S (2004) A photolabile hydrogel for guided three-dimensional cell growth and migration. Nat Mater 3:249-253.
Lutolf M P, Hubbell J A (2005) Synthetic biomaterials as instructive extracellular microenvironments for morphogenesis in tissue engineering. Nat Biotechnol 23:47-55.
Maniatopoulos, C., J. Sodek, A. H. Melcher. Bone formation in vitro by stromal cells obtained from bone marrow of young adult rats. Cell Tissue Res 254:317-330, 1988.
Mankani M H, Kuznetsov S A, Shannon B, Nalla R K, Ritchie R O, Qin Y, Robey P G (2006) Canine cranial reconstruction using autologous bone marrow stromal cells. Am J Pathol 168:542-550.
Mankani M H, Kuznetsov S A, Wolfe R M, Marshall G W, Robey P G (2006) In Vivo Bone Formation by Human Bone Marrow Stromal Cells: Reconstruction of the mouse calvarium and mandible. Stem Cells (In press).
Mao J J (2002) Mechanobiology of craniofacial sutures. J Dent Res 81:810-816.
Mao J J (2005a) Calvarial development: cells and mechanics. Curr Opin Orthop 16:331-337.
Mao J J (2005b) Stem cell driven regeneration of synovial joint. Biol Cell 97:289-301.
Mao J J, Giannobile W V, Helms J A, Hollister S J, Krebsbach P H, Longaker M T, Shi S (2006) Craniofacial tissue engineering by stem cells. J Dent Res (In press).
Mao J J, Nah H D (2004) Growth and development: hereditary and mechanical modulations. Am J Orthod Dentofac Orthop 125:676-689
Mao J J, Rahemtulla F, Scott P G (1998) Proteoglycan expression in articular tissues of the rat temporomandibular joint in response to bite raise. J Dent Res 77:1520-1528.
Mao J J, Wang X, Kopher R A (2003) Suture biomechanics: implications on craniofacial orthopedics. Angle Orthod 73:128-135.
Mao J J, Wang X, Kopher R A, Nudera J A, Mooney M P (2002) Strain induced osteogenesis in the cranial suture upon controlled delivery of low-frequency cyclic forces. Front Biosci 7:a246-252.
Marion N R, Liang W, Reilly G C, Day D E, Rahaman M N, Mao J J (2005) Borate glass supports the in vitro osteogenic differentiation of human mesenchymal stem cells. Mech Adv Materials Struct 12:1-8.
Marion N R, Mao J J (2006) Mesenchymal stem cells. In Robert Lanza, Irina Klimanskaya (Eds). Methods in Enzymology. Elsevier/Academic Press (In press).
Markopoulou, C. E., I. A. Vrotsos, H. N. Vavouraki, X. E. Dereka, Z. S. Mantzavinos Human periodontal ligament cell responses to recombinant human bone morphogenetic protein-2 with and without bone allografts. J Periodontol 74:982-989, 2003.
Martens, P. J., S. J. Bryant, K. S. Anseth. Tailoring the degradation of hydrogels formed multivinyl poly (ethylene glycol) and poly (vinyl alcohol) macromers for cartilage tissue engineering. Biomacromolecules 4:283-292, 2003.
McKee J A, Banik S S, Boyer M J, Hamad N M, Lawson J H, Niklason L E, Counter C M (2003) Human arteries engineered in vitro. EMBO Rep 4:633-638.
Meinel L, Betz O, Fajardo R, Hofmann S, Nazarian A, Hilbe M, McCool J, Langer R, Vunjak-Novakovic G, Merkle H P, Rechenberg B, Kaplan D L, Kirker-Head C (2006) Silk-based biomaterials for the healing of critical-size long bone defects. Bone (In press)
Meinel L, Fajardo R, Hofmann S, Langer R, Chen J, Snyder B, Vunjak-Novakovic G, Kaplan D L (2005) Silk implants for healing critical size cranial defects. Bone 37:688-698.
Miranda P, Saiz E, Gryn K, Tomsia A P (2006) Sintering and robocasting of beta-tricalcium phosphate scaffolds for orthopaedic applications. Acta Biomater (In press).
Miura M, Gronthos S, Zhao M, Lu B, Fisher L W, Robey P G, Shi S (2003) SHED: stem cells from human exfoliated deciduous teeth. Proc Natl Acad Sci USA 100:5807-5812.
Moioli E K, Hong L, Guardado J, Clark P A, Mao J J (2006) Controlled release of TGFβ3 on early osteogenic differentiation of human mesenchymal stem cells. Tissue Eng 12:537-546.
Moioli E K, Mao J J (2006) Cell density and stem cell homing. Tissue Eng (In press).
Mondoro T H, Thomas J W, Henslee-Downey P J, Peterson C M (2005) NHLBI plans for the promise of cell-based therapies. Cytotherapy 7:317-327.
Mondrinos M J, Koutzaki S, Jiwanmall E, Li M, Dechadarevian J P, Lelkes P I, Finck C M (2006) Engineering three-dimensional pulmonary tissue constructs. Tissue Eng 12:717-728.
Mooney D J, Mazzoni C L, Breuer C, McNamara K, Hem D, Vacanti J P, Langer R (1996) Stabilized polyglycolic acid fibre-based tubes for tissue engineering. Biomaterials 17:115-124.
Motlagh D, Yang J, Lui K Y, Webb A R, Ameer G A (2006) Hemocompatibility evaluation of poly(glycerol-sebacate) in vitro for vascular tissue engineering. Biomaterials 27:4315-4324.
Murphy W L, Simmons C A, Kaigler D, Mooney D J (2004) Bone regeneration via a mineral substrate and induced angiogenesis. J Dent Res 83:204-210.
Muschler G F, Nakamoto C, Griffith L G (2004) Engineering principles of clinical cell-based tissue engineering. J Bone Joint Surg Am 86-A:1541-1558.
Nair L S, Bhattacharyya S, Laurencin C T (2004) Development of novel tissue engineering scaffolds via electrospinning. Expert Opin Biol Ther 4:659-668.
Nehrer, S., H. A. Breinan, A. Ramappa, H. P. Hsu, T. Minas, S. Shortkroff, C. B. Sledge, I. V. Yannas, M. Spector. Chondrocyte-seeded collagen matrices implanted in a chondral defect in a canine model. Biomaterials 19: 2313-2328, 1998.
Neubauer M, Hacker M, Bauer-Kreisel P, Weiser B, Fischbach C, Schulz M B, Goepferich A, Blunk T. (2005) Adipose tissue engineering based on mesenchymal stem cells and basic fibroblast growth factor in vitro. Tissue Eng. 2005 November-December; 11(11-12):1840-51.
Nevins, M., M. Camelo, M. L. Nevins, R. K. Schenk, S. E. Lynch. Periodontal regeneration in humans using recombinant human platelet-derived growth factor-BB (rhPDGF-BB) and allogenic bone. J Periodontol 74:1282-1292, 2003.
Nishimura, K., M. Hayashi, K. Matsuda, Y. Shigeyama, A. Yamasaki, A. Yamaoka. The chemoattractive potency of periodontal ligament, cementum and dentin for human gingival fibroblasts. J Periodontal Res 24:146-148, 1989.
Noden D M, Trainor P A (2005) Relations and interactions between cranial mesoderm and neural crest populations. J Anat 207:575-601.
Oberheim M C, Mao J J (2002). Bone strain patterns of the zygomatic complex in response to simulated orthopedic forces. J Dent Res 81:608-612.
Ochi K, Chen G, Ushida T, Gojo S, Segavth K, Tai H, Ueno K, Ohkawa H, Mod T, Yamaguchi A, Toyama Y, Hata J, Umezawa A (2003). Use of isolated mature osteoblasts in abundance acts as desired-shaped Bone regeneration in combination with a modified poly-DL-lactic-co-glycolic acid (PLGA)-collagen sponge. J Cell Physiol 194:45-53.
Ohgushi H, Caplan A I (1999) Stem cell technology and bioceramics: from cell to gene engineering. J Biomed Mater Res 48:913-927.
Ohgushi, H., A. I. Caplan. Stem cell technology and bioceramics: from cell to gene engineering. J Biomed Mater Res 48:913-927, 1999.
Osborne C S, Barbenel J C, Smith D, Savakis M, Grant M H (1998). Investigation into the tensile properties of collagen/chondroitin-6-sulphate gels: the effect of crosslinking agents and diamines. Med Biol Eng Comput. 36:129-134.
Osyczka, A. M. and P. S. Leboy. BMP regulation of early osteoblast genes in human marrow stromal cells is mediated by ERK and PI3-K signaling. Endocrinology 146:3428-3437, 2005.
Pacifici M, Koyama E, Iwamoto M (2005) Mechanisms of synovial joint and articular cartilage formation: recent advances, but many lingering mysteries. Birth Defects Res C Embryo Today 75:237-248.
Parfitt A M, Drezner M K, Glorieux F H, Kanis J A, Malluche H, Meunier P J, Ott S M, Recker R R (1987) Bone histomorphometry: standardization of nomenclature, symbols, and units. Report of the ASBMR Histomorphometry Nomenclature Committee. J Bone Miner Res 2:595-610.
Park Y J, Lee Y M, Lee J Y, Seol Y J, Chung C P, Lee S J (2000) Controlled release of platelet-derived growth factor-BB from chondroitin sulfate-chitosan sponge for guided bone regeneration. J Control Rel 67:385-394.
Parker G C, Anastassova-Kristeva M, Eisenberg L M, Rao M S, Williams M A, Sanberg P R, English D; Editorial Board of Stem Cells and Development (2005) Stem cells: shibboleths of development, part II: Toward a functional definition. Stem Cells Dev 14:463-469.
Passegue E, Wagers A J (2006) Regulating quiescence: new insights into hematopoietic stem cell biology. Dev Cell 10:415-417.
Patel R V, Mao J J (2003). Microstructural and elastic properties of the extracellular matrices of the superficial zone of neonatal articular cartilage by atomic force microscopy. Front Biosci 8:a123-a130.
Pei M, Solchaga L A, Seidel J, Zeng L, Vunjak-Novakovic G, Caplan A I, Freed L E (2002). Bioreactors mediate the effectiveness of tissue engineering scaffolds. FAS EB J 16:1691-1694.
Pelissier P, Villars F, Mathoulin-Pelissier S, Bareille R, Lafage-Proust M H, Vilamitjana-Amedee J (2003) Influences of vascularization and osteogenic cells on heterotopic bone formation within a madreporic ceramic in rats. Plast Reconstr Surg 111:1932-1941.
Peptan I A, Hong L, Mao J J (2006) Comparison of osteogenic potentials of visceral and subcutaneous adipose-derived cells of rabbits. Plastic Reconstruct Surg 117:1462-1470.
Pereira, R. F., K. W. Halford, M. D. O'Hara, D. B. Leeper, B. P. Sokolov, M. D. Pollard, O. Bagasra, D. J. Prockop. Cultured adherent cells from marrow can serve as long-lasting precursor cells for bone, cartilage, and lung in irradiated mice. Proc. Natl. Acad. Sci. U.S.A. 92: 4857-4861, 1995.
Peters M C, Polyerini P J, Mooney D J (2002) Engineering vascular networks in porous polymer matrices. J Biomed Mater Res 60:668-678.
Pitaru, S., S. A. Narayanan, S. Olson, N. Savion, H. Hekmati, I. Alt, Z. Metzger. Specific cementum attachment protein enhances selectively the attachment and migration of periodontal cells to tooth root surfaces. J Periodontal Res 30:360-368, 1995.
Pittenger M F, Mackay A M, Beck S C, Jaiswal R K, Douglas R, Mosca J D, Moorman M A, Simonetti D W, Craig S, Marshak D R (1999). Multilineage potential of adult human mesenchymal stem cells. Science 284:143-147.
Pittenger, M. F., A. M. Mackay, S. C. Beck, R. K. Jaiswal, R. Douglas, J. D. Mosca, M. A. Moorman, D. W. Simonetti, S. Craig, D. R. Marshak. Multilineage potential of adult human mesenchymal stem cells. Science 284, 143-147, 1999.
Ponticiello M S, Schinagl R M, Kadiyala S, Barry F P (2000). Gelatin-based resorbable sponge as a carrier matrix for human mesenchymal stem cells in cartilage regeneration therapy. J Biomed Mater Res 52:246-255.
Posnick J C (2000) Craniosynostosis and the craniofacial dysostosis syndromes: current surgical management In: Cohen M M Jr and MacLean R E (Eds). Craniosynostosis: Diagnosis, Evaluation and Management.
Pradeep, A. R. and B. V. Karthikeyan. Tissue engineering: prospect for regenerating periodontal tissues. Indian J Dent Res 14:224-229, 2003. 45. Ripamonti, U. Bone induction by BMPs/OPs and related family members in primates. J. Bone Joint Surg. Am 83: S116-127, 2001.
Radhakrishnan P, Lewis N T, Mao J J (2004) Zone-specific micromechanical properties of the extracellular matrices of growth plate cartilage. Ann Biomed Eng 32:284-291.
Radhakrishnan P, Mao J J (2004) Nanomechanical properties of facial sutures and sutural mineralization front. J Dent Res 83:470-475.
Rahaman M N, Mao J J (2005) Stem cell based composite tissue constructs for regenerative medicine. Biotechnol & Bioeng 91:261-284.
Rajashekhar G, Willuweit A, Patterson C E, Sun P, Hilbig A, Breier G, Helisch A, Clauss M (2006) Continuous endothelial cell activation increases angiogenesis: evidence for the direct role of endothelium linking angiogenesis and inflammation. J Vasc Res 43:193-204.
Rivas R, Shapiro F (2002) Structural stages in the development of the long bones and epiphyses: a study in the New Zealand white rabbit. J Bone Joint Surg Am 84-A:85-100.
Rose, L. F., B. L. Mealey, R. J. Genco, D. W. Cohen. Medicine, Surgery, and Implants, periodontics: ISBN 0-8016-7978-8. Elsevier Mosby, 238-239; 573-601, 2004.
Rotte, N., J. Aigner, A. Naumann, H. Planck, C. Hammer, G. Burmester, M. Sittinger. Cartilage reconstruction in head and neck surgery: Comparison of resorbable polymer scaffolds for tissue engineering of human septal cartilage. J Biomed Mater Res 42: 347-356, 1998.
Rubin C T, Lanyon L E (1987). Osteoregulatory nature of mechanical stimuli: function as a determinant for adaptive remodeling in bone. J Orthop Res; 5:300-310.
Ruiz de Almodovar C, Luttun A, Carmeliet P (2006) An SDF-1 trap for myeloid cells stimulates angiogenesis. Cell 124:175-89.
Satomura, K., P. Krebsbach, P. Bianco, P. G. Robey. Osteogenic imprinting upstream of marrow stromal cell differentiation. J Cell Biochem. 78:391-403, 2000.
Schmitt J M, Hwang K, Winn S R, Hollinger J O (1999) Bone morphogenetic proteins: an update on basic biology and clinical relevance. J Orthop Res 17:269-278.
Schultz G, Rotatori D S, Clark W (1991). EGF and TGF-alpha in wound healing and repair. J Cell Biochem 45:346-352.
Sculean, A., G. C. Chiantella, P. Windisch, N. Donos. Clinical and histologic evaluation of human intrabony defects treated with an enamel matrix protein derivative (Emdogain). Int J Periodontics Restorative Dent 20:374-381, 2000.
Sculean, A., N. Donos, M. Brecx, T. Carring, E. Reich. Healing of fenestration-type defects following treatment with guide tissue regeneration or enamel matrix proteins. An experimental study in monkeys. Clin Oral Invest 4:50-56, 2000.
Seo B M, Miura M, Gronthos S, Bartold P M, Batouli S, Brahim J, Young M, Robey P G, Wang C Y, Shi S. (2004) Investigation of multipotent postnatal stem cells from human periodontal ligament. Lancet 364:149-155.
Seruya M, Shah A, Pedrotty D, du Laney T, Melgiri R, McKee J A, Young H E, Niklason L E (2004) Clonal population of adult stem cells: life span and differentiation potential. Cell Transplant 13:93-101.
Shame E E 3rd, Teleron A A, Li B, Price J, Sands M S, Alford K, Young P P (2006) The origin and in vivo significance of murine and human culture-expanded endothelial progenitor cells. Am J Pathol 168:1710-1721.
Shea L D, Wang D, Franceschi R T, Mooney D J (2000). Engineered bone development from a pre-osteoblast cell line on three-dimensional scaffolds. Tissue Eng 6:605-617.
Shen W, Li Y, Huard J (2005) Musculoskeletal gene therapy and its potential use in the treatment of complicated musculoskeletal infection. Infect Dis Clin North Am 19:1007-1022.
Sherman R, Chapman W C, Hannon G, Block J E (2001). Control of bone bleeding at the sternum and iliac crest donor sites using a collagen-based composite combined with autologous plasma: results of a randomized controlled trial. Orthopedics 24:137-141.
Shi Q, Rafii S, Wu M H, Wijelath E S, Yu C, Ishida A, Fujita Y, Kothari S, Mohle R, Sauvage L R, Moore M A, Storb R F, Hammond W P (1998) Evidence for circulating bone marrow-derived endothelial cells. Blood 92:362-367.
Shi O, Ratii S, Wu M H, Wijelath E S, Yu C, Ishida A, Fujita Y, Kothari S, Mohle R, Sauvage L R, Moore M A, Storb R F, Hammond W P (1998) Evidence for circulating bone marrow-derived endothelial cells. Blood. 92:362-367.
Shi S, Gronthos S, Chen S, Reddi A, Counter C M, Robey P G, Wang C Y (2002). Bone formation by human postnatal bone marrow stromal stem cells is enhanced by telomerase expression. Nat Biotechnol 20:587-591.
Shi Y J, Shen R N, Lu L, Broxmeyer H E (1994) Comparative analysis of retroviral-mediated gene transduction into CD34+ cord blood hematopoietic progenitors in the presence and absence of growth factors. Blood Cells 20:517-524.
Shih C, Digiusto D, Forman S J (2000) Ex vivo expansion of transplantable human hematopoietic stem cells. J Hematotherapy & Stem Cell Res 9:621-628.
Shimizu, Y. Tissue engineering for soft tissues. In: Ikeda, Y., ed. Tissue Engineering for Therapeutic Use 1. Tokyo: Elsevier, pp. xv-xvii, 1998.
Shive M S, Anderson J M (1997). Biodegradation and biocompatibility of PLA and PLGA microspheres. Adv Drug Deliv Rev 13; 28(1):5-24.
Sikavitsas V I, Temenoff J S, Mikos A G. Biomaterials and bone mechanotransduction. Biomaterials 22:2,581-2,593, 2001.
Sokal R R, Rohlf F J (1981) Biometry: The Principles and Practice of Statistics in Biological Research. 2nd Ed., p. 672 and pp. 583-591. W.H. Freeman & Co., New York.
Somerman, M. J., B. Shroff, W. S. Argraves, G. Morrison, A. M. Craig, D. T. Denhardt, R. A. Foster, J. J. Sauk. Expression of attachment proteins during cementogenesis. J Biol Buccale 18:207-214, 1990.
Stahl A, Wu X, Wenger A, Klagsbrun M, Kurschat P (2005) Endothelial progenitor cell sprouting in spheroid cultures is resistant to inhibition by osteoblasts: a model for bone replacement grafts. FEBS Lett 579:5338-5842.
Stevens M M, Marini R P, Schaefer D, Aronson J, Langer R, Shastri V P (2005) In vivo engineering of organs: the bone bioreactor. Proc Natl Aced Sci USA 102:11450-11455.
Stosich M S, Bastian B, Clark P A, Marion N M, Mao J J (2006) Engineered neovascularization in hydrogel promotes de novo adipogenesis from human mesenchymal stem cells. FASEB J Express Manuscript submission invited (FASEBJ/2006/052746).
Stosich M S, Mao J J (2005) Stem cell based soft tissue grafts for plastic and reconstructive surgeries. Sem Plastic Surg 19:251-260.
Stosich M S, Mao J J (2006) Adipose tissue engineered from adult human stem cells: therapeutic applications in reconstructive and plastic surgeries. Plast Reconstruct Surg (In press).
Sundaramurthy S, Mao J J (2006) Mechanical modulation of endochondral development in the distal femoral condyle. J Orthop Res 24:229-241.
Takai E, Costa K D, Shaheen A, Hung C T, Guo X E (2005) Osteoblast elastic modulus measured by atomic force microscopy is substrate dependent. Ann Biomed Eng 33:963-971.
Talwar, R., L. De. Silvio, F. J. Heghes, G. N. King. Effects of carrier release kinetics on bone morphogenic protein-2-induced periodontal ligament regeneration in vivo. J Clin Periodontol 28:340-347, 2001.
Tang M, Mao J J (2006) Matrix and Gene Expression in the Rat Cranial Base Growth Plate. Cell Tiss Res 324:467-474.
Tang, M. H., H. Takita, T. Kohgo, M. Iizuka, Y. Doi, Y. Kuboki. BMP-induced osteogenesis with two geometrically different biodegradable carbonate apatites. J Hard Tissue Biology 10: 7-16, 2001.
Tanihara M, Suzuki Y, Yamamoto E, Noguchi A, Mizushima Y (2001) Sustained release of basic fibroblast growth factor and angiogenesis in a novel covalently crosslinked gel of heparin and alginate. J Biomed Mater Res 56:216-221.
Tapadia M D, Cordero D R, Helms J A (2005) It's all in your head: new insights into craniofacial development and deformation. J Anat 207:461-477.
Thurston G (2002) Complementary actions of VEGF and angiopoietin-1 on blood vessel growth and leakage. J Anat 200:575-580.
Tomkoria S, Patel R V, Mao J J (2004) Heterogeneous micromechanical properties of articular and zonal regions of the rabbit radius condyle by atomic force microscopy. Med Eng Phys 26:815-22.
Troken A, Wan L C, Marion N R, Mow V C, Mao J J (2006) Properties of Cartilage and Menisci. Wiley Encyclopedia of Medical Devices and Instrumentation (In press).
Troken, Mao J J (2006) Cell density in TMJ tissue engineering. J Eng Med (In press).
Trumpp W A (2006) Bone-marrow haematopoietic-stem-cell niches. Nat Rev Immunol 6:93-106.
Tsang V L, Bhatia S N (2004) Three-dimensional tissue fabrication. Adv Drug Deliv Rev 56:1635-1647.
Tuan R S (2004) Biology of developmental and regenerative skeletogenesis. Clin Orthop Relat Res 427 (Suppl):S105-117.
Tuli, R., S, Nandi, W. J. Li, S. Li, X. Huang, P. A. Manner, P. Laquerrieer, U. Noth, D. J. Hall, R. S. Tuan. Huaman mesenchymal progenitor cell-based tissue engineering of a single-unit osteochondral construct. Tissue Engineering 10:1169-1179, 2004.
Udani V M (2006) The continuum of stem cell transdifferentiation: possibility of hematopoietic stem cell plasticity with concurrent CD45 expression. Stem Cells Dev 15:1-3.
Valeski J E, Baldwin A L (2003) Role of the actin cytoskeleton in regulating endothelial permeability in venules. Microcirculation 10:411-420.
Velegol D, Lanni F (2001) Cell traction forces on soft biomaterials. I. Microrheology of type I collagen gels. Biophys J 81:1786-1792.
Vij K, Mao J J (2006) Geometry and cell density of rat craniofacial sutures during early postnatal development and upon in vivo cyclic loading. Bone 38:722-730.
Volk S W, Leboy P S (1999) Regulating the regulators of chondrocyte hypertrophy. J Bone Miner Res 14:483-486.
Vunjak-Novakovic G, Obradovic B, Martin I, Freed L E (2002). Bioreactor studies of native and tissue engineered cartilage. Biorheol 39:259-268.
Vunjak-Novakovic G., Martin I., Obradovic B., Treppo S, Grodzinsky A. J., Langer R., Freed L (1999) Bioreactor cultivation conditions modulate the composition and mechanical properties of tissue engineered cartilage. J Orthop Res 17:130-138.
Wang X, Mao J J (2002) Accelerated chondrogenesis of the rabbit cranial base growth plate upon oscillatory mechanical stimuli. J Bone and Miner Res 17:457-462.
Weinand C, Pomerantseva I, Neville C M, Gupta R, Weinberg E, Madisch I, Shapiro F, Abukawa H, Troulis M J, Vacanti J P (2006) Hydrogel-beta-TCP scaffolds and stem cells for tissue engineering bone. Bone 38:555-563.
Wenger A, Stahl A, Weber H, Finkenzeller G, Augustin H G, Stark G B, Kneser U (2004) Modulation of in vitro angiogenesis in a three-dimensional spheroidal coculture model for bone tissue engineering. Tissue Eng 10:1536-1547.
Wikesjo U. M., M. Qahash, R. C. Thomson, A. D. Cook, M. D. Rohrer, J. M. Wozney, W. R. Hardwick. Space-providing expanded polytetrafluoroethylene devices define alveolar augmentation at dental implants induced by recombinant human bone morphogenetic protein 2 in an absorbable collagen sponge carrier. Clin Implant Dent Relat Res 5:112-123, 2003A.
Wikesjo, U. M., A. V. Xiropaidis, R. C. Thomson, A. D. Cook, K. A. Selvig, W. R. Hardwick. Periodontal repair in dogs: rhBMP-2 significantly enhances bone formation under provisions for guided tissue regeneration. J Clin Periodontol 30:705-714, 2003B.
Williams C G, Malik A N, Kim T K, Manson P N, Elisseeff J H (2005) Variable cytocompatibility of six cell lines with photoinitiators used for polymerizing hydrogels and cell encapsulation. Biomaterials 26:1211-1218.
Wilson A, Trumpp A (2006) Bone-marrow haematopoietic-stem-cell niches. Nat Rev Immunol 6:93-106.
Xin X J, Hussein M A, Mao J J (2006) Human mesenchymal stem cells and osteogenic derivatives differentiate on PLGA nanofibers in vitro: molecular and nanotopographic characterization. Biomaterials (In press).
Yamanouchi K, Satomura K, Gotoh Y, Kitaoka E, Tobiume S, Kume K, Nagayama M (2001) Bone formation by transplanted human osteoblasts cultured within collagen sponge with dexamethasone in vitro. J Bone Miner Res 16:857-867.
Yeoh J S, van Os R, Weersing E, Ausema A, Dontje B, Vellenga E, de Haan G (2006) Fibroblast growth factor-1 and 2 preserve long-term repopulating ability of hematopoietic stem cells in serum-free cultures. Stem Cells (In press).
Yin T, Li L (2006) The stem cell niches in bone. J Clin Invest 116:1195-1201.
Yoo, J. U. and B. Johnstone. The role of osteochondral progenitor cells in fracture repair. Clin Orthop Relat Res 355 Suppl:S73-81, 1998.
Young, R. G., D. L. Butler, W. Weber, A. I. Caplan, S. L. Gorden, D. J. Fink. Use of mesenchymal stem cells in a collagen matrix for Achilles tendon repair. J Orthop Res 16: 406-413, 1998.
Yourek G, Alhadlaq A, Patel R, McCormick S, Reilly G C, Mao J J (2004) Nanophysical properties of living cells: The Cytoskeleton. In Dutta M, Stroscio M (Eds) Biological Nanostructures and Applications of Nanostructures in Biology: Electrical, Mechanical, and Optical Properties. New York, N.Y., Kluwer Academic Publishing, pp. 69-97.
Zabka A G, Pluhar G E, Edwards R B 3rd, Manley P A, Hayashi K, Heiner J P, Kalscheur V L, Seeherman H J, Markel (2001). Histomorphometric description of allograft bone remodeling and union in a canine segmental femoral defect model: a comparison of rhBMP-2, cancellous bone graft, and absorbable collagen sponge. J Orthop Res 19:318-327.
Zhang N, Mustin D, Reardon W, Almeida A D, Mozdziak P, Mrug M, Eisenberg L M, Sedmera D (2006) Blood-borne stem cells differentiate into vascular and cardiac lineages during normal development. Stem Cells Dev 15:17-28.
Zhou X, Stuart A, Dettin L E, Rodreguez G, Hoel B, Gallicano G I (2004) Desmoplakin is required for microvascular tube formation in culture. J Cell Sci 117:3129-3140.
Zisch A H (2004) Tissue engineering of angiogenesis with autologous endothelial progenitor cells. Curr Opin Biotechnol 15:424-429.
Zuk P A, Zhu M, Mizuno H, Huang J, Futrell J W, Katz A J, Benhaim P, Lorenz H P, Hedrick M H (2001) Multilineage cells from human adipose tissue: implications for cell-based therapies. Tissue Eng 7:211-228.

1. A tissue module comprising:
(a) a biocompatible matrix;
(b) vascular progenitor cells; and
(c) tissue progenitor cells;
wherein the module is ex vivo, or at least one of (a), (b), or (c) is heterologous to a vertebrate recipient.

2-6. (canceled)

7. The tissue module of claim 1, wherein the tissue progenitor cells are selected from the group consisting of mesenchymal stem cells (MSC), MSC-derived cells, osteoblasts, chondrocytes, myocytes, adipocytes, neurons, glial cells, fibroblasts, cardiomyocytes, liver cells, kidney cells, bladder cells, beta-pancreatic islet cell, odontoblasts, dental pulp cells, periodontal cells, tenocytes, lung cells, cardiac cells, and a combination thereof.

8. The tissue module of claim 7, wherein the tissue progenitor cells are fibroblasts selected from the group consisting of interstitial fibroblasts, tendon fibroblasts, ligament fibroblasts, periodontal fibroblasts, and craniofacial fibroblasts.

9. The tissue module of claim 7 wherein the tissue progenitor cells are MSC chondrocytes.

10. The tissue module of claim 7 wherein the tissue progenitor cells are MSCs.

11. The tissue module of claim 1 wherein the vascular progenitor cells are selected from the group consisting of hematopoietic stem cells (HSC), HSC endothelial cells, blood vascular endothelial cells, lymph vascular endothelial cells, cultured endothelial cells, primary culture endothelial cells, bone marrow stem cells, cord blood cells, human umbilical vein endothelial cell (HUVEC), lymphatic endothelial cell, endothelial progenitor cell, stem cells that differentiate into an endothelial cells, smooth muscle cells, interstitial fibroblasts, and myofibroblasts.

12. The tissue module of claim 11 wherein the vascular progenitor cells are HSCs.

13. The tissue module of claim 11 wherein the vascular progenitor cells are HSC endothelial cells.

14. The tissue module of claim 1 wherein the matrix comprises a material selected from the group consisting of fibrin, fibrinogen, a collagen, a polyorthoester, a polyvinyl alcohol, a polyamide, a polycarbonate, a polyvinyl pyrrolidone, a marine adhesive protein, a cyanoacrylate, a polymeric hydrogel, and a combination thereof.

15. The tissue module of claim 1 wherein the matrix comprises a polymeric hydrogel.

16. (canceled)

17. The tissue module of claim 1 wherein the matrix comprises a plurality of physical channels having an average diameter of at least about 0.1 mm up to about 50 mm.

18. (canceled)

19. The tissue module of claim 1, wherein the matrix further comprises a growth factor.

20. The tissue module of claim 19 wherein the growth factor is an angiogenic growth factor.

21. The tissue module of claim 19 wherein the growth factor is selected from the group consisting of bFGF, VEGF, PDGF, TGFβ, and a combination thereof.

22-25. (canceled)

26. A method of treating a tissue or organ defect in a subject, the method comprising grafting the module of claim 1 into the defect.

27-30. (canceled)

31. The method of claim 26 wherein the defect is a bone, adipose, bladder, brain, breast, osteochondral junction, central nervous system, spinal cord, peripheral nerve, glia, esophagus, fallopian tube, heart, pancreas, intestines, gallbladder, kidney, liver, lung, ovaries, prostate, spleen, skeletal muscle, skin, stomach, testes, thymus, thyroid, trachea, urogenital tract, ureter, urethra, interstitial soft tissue, periosteum, periodontal tissue, cranial sutures, hair follicles, oral mucosa, or uterus defect.

32. The method of claim 26 wherein the defect is a bone defect.

33. The method of claim 26 wherein the tissue defect is a adipose tissue defect.

34. The method of claim 32, wherein the module comprises VEGF, PDGF, mesenchymal stem cells or cells derived therefrom, and hematopoetic stem cells or cells derived therefrom.
